(19) United States
(12) Patent Application Publication
(10) Pub. No.: US 2009/0239793 A1
(43) Pub. Date: Sep. 24, 2009

(54) Polynucleotides and polypeptides associated with trophoblast cell death, differentiation, invasion, and/or cell fusion and turnover

(76) Inventors: Isabella Caniggia, Toronto (CA); Martin Post, Toronto (CA); Nima Soleymanlou, Thornhill (CA); Andrea Jurisicova, Toronto (CA)

Correspondence Address: HOWSON & HOWSON LLP, 501 OFFICE CENTER DRIVE, SUITE 210, FORT WASHINGTON, PA, 19034, US

(21) Appl. No.: 11/663,678

(22) PCT Filed: Sep. 23, 2005
(86) PCT No.: PCT/CA05/01455

§ 371 Date: May 28, 2009

Related U.S. Application Data
(60) Provisional application No. 60/612,709, filed on Sep. 24, 2004.
(60) Provisional application No. 60/651,742, filed on Feb. 10, 2005.

(51) Int. Cl. A61K 38/16; C12N 15/11; C12N 15/00; C07K 14/00; C07K 16/18; C12Q 1/68; G01N 33/68; C40B 40/08; G01N 33/53; A61K 31/7088

(52) U.S. Cl. 514/12; 536/23.5; 435/320.1; 530/350; 530/387.9; 435/6; 436/86; 506/17; 435/7.21; 514/44 R

(57) ABSTRACT

The invention relates to polypeptides and polynucleotides associated with trophoblast cell death, differentiation, invasion, and/or cell fusion and turnover, and uses of same in the prevention, diagnosis and treatment of conditions requiring regulation of trophoblast cell death, differentiation, invasion, and/or cell fusion and turnover. In particular aspects, diagnostic methods are disclosed for evaluating conditions such as preeclampsia utilizing matador polypeptides and polynucleotides encoding same.

### FIELD OF THE INVENTION

The invention relates to polypeptides and polynucleotides associated with trophoblast cell death, differentiation, invasion, and/or cell fusion and turnover, and uses of same in the prevention, diagnosis and treatment of conditions requiring regulation of trophoblast cell death, differentiation, invasion, and/or cell fusion and turnover.

### BACKGROUND OF THE INVENTION

Preeclampsia, a complex and serious disorder of human pregnancy, is presently the leading cause of fetal and maternal morbidity and mortality worldwide, affecting approximately 5-7% of all pregnancies (1). Clinical diagnosis and symptoms are based on sudden onset of hypertension accompanied by proteinuria and edema. Although the etiology and pathophysiology of this disease remain unclear, it is accepted that the presence of the placenta, not the fetus, is at the origin of this disease (2).

A key histopathologic feature of preeclampsia is the shallow trophoblast invasion of the maternal site. Doppler studies have demonstrated that the preeclamptic feto-maternal interface experiences an abnormally elevated myometrial vascular resistance and decreased utero-placental perfusion as a result of incomplete trophoblast-mediated remodeling of myometrial spiral arteries, which retain their vasoactive characteristic (3,4). In this condition, inadequate placental perfusion leads to oxidative stress, placental hypoxia/ischaemia and, in severe cases, infarctions (5,6,7).

It is postulated that in preeclampsia, excessive placental shedding of syncytiotrophoblast microfragments (STBM), also known as syncytial knots (SK), in the maternal circulation may directly contribute to the initiation of maternal inflammation culminating in systemic endothelial cell damage (6). The source of this excess foreign fetal debris is the result of increased turnover of trophoblast cells. The renewal of placental syncytium, known to be mediated via apoptosis, is a physiological process observed throughout pregnancy, and is believed to be initiated in the underlying mononucleated progenitor cytotrophoblast layer (8). Importantly, trophoblast apoptosis as well as syncytial shedding have been demonstrated to be significantly increased in preeclampsia (9,10,11,12,13). The exact mechanisms causing these events remain unclear.

Members of the Bcl-2 family have been shown to be important intrinsic regulators of apoptosis in developmental processes as well as numerous diseases (14,15). Previous studies have reported the placental expression of the anti-apoptotic molecules Bcl-2 and Mcl-1 in the syncytium as well as in the cytotrophoblast layer of placental tissues from normal pregnancies (8,16). While some studies have reported that Bcl-2 expression is unchanged in placentae of pregnancies complicated by preeclampsia (17,18), others have reported a decreased expression of this molecule in severe preeclampsia (19). Both pro-apoptotic molecules Bax and Bak are expressed in villous cytotrophoblast cells and in syncytium of normal placentae (16), but their expression is not different between placentae of preeclamptic and control subjects (17,18). Mtd/Bok (Mtd: Matador/Bok: Bcl-2 ovarian killer) belongs to the multi-domain pore-forming subfamily of pro-apoptotic Bcl-2 family members including Bax and Bak. Previous findings have reported that the expression of Mtd is greatest in reproductive tissues (20). To date, two splice isoforms of the Mtd gene have been characterized. The full-length pro-apoptotic protein, Mtd-L, that mainly interacts with Mcl-1, and a shorter pro-apoptotic transcript, Mtd-S. The function of this short isoform, resulting from fusion of BH3 and BH1 domains, cannot be antagonized by any known anti-apoptotic Bcl-2 family member (21).

### SUMMARY OF THE INVENTION

A novel splice variant of the Matador/Bok: Bcl-2 ovarian killer (Mtd) gene, a 546 bp transcript in which the second of six Mtd exons is skipped, has been identified and characterized. The splice variant was found to have a distinctive developmental expression profile; its over-expression is unique to pregnancies complicated by severe early onset preeclampsia; it is a pro-apoptotic molecule involved in trophoblast cell death; and, its expression is increased under conditions of reduced oxygenation and oxidative stress.

The novel Mtd-P polypeptide described herein is referred to as “Mtd-P” or “Mtd-P Polypeptide”. A polynucleotide encoding the polypeptide described herein is referred to as a “Mtd-P Polynucleotide”. Broadly stated, the present invention relates to an isolated Mtd-P Polypeptide, in particular a polypeptide comprising the amino acid sequence of SEQ ID NO: 1, and truncations, analogs, sequences with sequence identity, and homologs thereof (collectively referred to herein as “Mtd-P Related Polypeptides”.)

The invention also contemplates an isolated polynucleotide encoding a Mtd-P Related Polypeptide, in particular a polypeptide comprising an amino acid sequence of SEQ ID NO: 1, an isolated polynucleotide comprising the nucleic acid sequence of SEQ ID NO: 2, conservative variants of SEQ ID NO: 2, and complements thereof, and fragments or polynucleotides that hydridize to the nucleic acid sequence that are unique to SEQ ID. NO. 2 (“Mtd-P Related Polynucleotides”).

In addition, the invention provides an isolated polynucleotide comprising the coding region of a Mtd-P Related Polypeptide operably linked to a regulatory element (e.g. a hypoxia-insensitive promoter).

The Mtd-P Related Polynucleotides of the invention may be inserted into appropriate expression vectors, e.g., vectors that contain the necessary elements for the transcription and translation of the inserted coding sequence or the translation of the inserted transcript. Accordingly, recombinant expression vectors adapted for transformation of a host cell may be constructed which comprise a polynucleotide of the invention and at least one regulatory element (e.g. transcription element and/or translation element) linked to the polynucleotide.

The recombinant expression vector can also be used to prepare transformed host cells expressing Mtd-P Related Polypeptides. Therefore, the invention further provides host cells containing a recombinant vector of the invention. The invention also contemplates transgenic non-human mammals whose germ cells and somatic cells contain a recombinant vector comprising a polynucleotide of the invention, in particular, one which encodes an analog of a Mtd-P Polypeptide or a truncation of a Mtd-P Polypeptide.

The invention further provides a method for preparing Mtd-P Related Polypeptides utilizing the isolated polynucleotides of the invention. In an embodiment a method for preparing a Mtd-P Related Polypeptide is provided comprising (a) transferring a recombinant expression vector of the invention into a host cell; (b) selecting transformed host cells from untransformed host cells; (c) culturing a selected transformed host cell under conditions which allow expression of the Mtd-P Related Polypeptide; and (d) isolating the Mtd-P Related Polypeptide.

The invention further contemplates antibodies having specificity against an epitope of a Mtd-P Related Polypeptide of the invention. Antibodies may be labelled with a detectable substance and used to detect polypeptides of the invention in tissues and cells. Antibodies may have particular use in therapeutic applications, for example to react with cells, and in conjugates and immunotoxins as target selective carriers of various agents which have therapeutic effects including chemotherapeutic drugs, toxins, immunological response modifiers, enzymes, and radioisotopes.

The invention also permits the construction of nucleotide probes that are unique to the polynucleotides of the invention and/or to polypeptides of the invention. Therefore, the invention also relates to a probe comprising a polynucleotide of the invention, or a nucleic acid encoding a polypeptide of the invention, or a part thereof. The probe may be labelled, for example, with a detectable substance and it may be used to select from a mixture of nucleotide sequences a polynucleotide of the invention including polynucleotides coding for a protein which displays one or more of the properties of a polypeptide of the invention (and further including polynucleotides translatable into a protein which displays one or more properties of a polypeptide of the invention). In an aspect, a probe may be used to mark preeclamptic tissues.

The invention also provides antisense polynucleotides, e.g., a mRNA or DNA strand in the reverse orientation to a sense molecule encoding a Mtd-P Related Polypeptide. An antisense polynucleotide may be used to inhibit transcription of mRNA and thus production of Mtd-P Related Polypeptides. This can have particular application in a condition involving a Mtd-P Related Polypeptide (e.g., preeclampsia) where an antisense polynucleotide may inhibit the development of the condition.

The invention also provides mRNA-interfering complementary RNA (“micRNA”), e.g. a nucleic acid strand in the reverse orientation to a mRNA transcript for a Mtd-P Related Polynucleotide. A micRNA molecule may be used to modulate production of Mtd-P Related Polynucleotides.

The invention provides a method for evaluating a compound for its ability to modulate the biological activity of a Mtd-P Related Polypeptide of the invention. For example, a substance which inhibits or enhances the interaction of the polypeptide and a substance which binds to the polypeptide may be evaluated. In one embodiment, the method comprises providing a known concentration of a Mtd-P Related Polypeptide, with a substance which binds to the polypeptide and a test compound under conditions which permit the formation of complexes between the substance and polypeptide, and removing and/or detecting complexes.

Compounds which modulate the biological activity of a polypeptide of the invention may also be identified using the methods of the invention by comparing the pattern and level of expression of the polypeptide of the invention in tissues and cells, in the presence, and in the absence of the compounds.

The invention further relates to a method of selecting a substance that modulates trophoblast cell death, differentiation, invasion, and/or cell fusion and turnover comprising assaying for a substance that inhibits or stimulates a Mtd polypeptide or polynucleotide encoding same. The substances may be used in the methods of the invention to regulate trophoblast invasion.

The polypeptides of the invention, antibodies, antisense polynucleotides, micRNA molecules, and substances and compounds identified using the methods of the invention, may be used to modulate the biological activity of Mtd-P Related Polypeptides, and they may be used in the diagnosis, prevention, and treatment of conditions involving a Mtd-P Related Polypeptide, and conditions associated with trophoblast cell death, differentiation, invasion, and/or cell fusion and turnover such as preeclampsia in a subject. Accordingly, the substances and compounds may be formulated into compositions for administration to individuals at risk for or suffering from such conditions.

Therefore, the present invention relates to a composition comprising one or more of a polypeptide or polynucleotide of the invention, or a substance, agent, or compound identified using the methods of the invention, and a pharmaceutically acceptable carrier, excipient or diluent.

The invention also relates to a composition adapted for modulating trophoblast cell death, differentiation, invasion, and/or cell fusion and turnover comprising a substance which inhibits or stimulates a Mtd polypeptide or Mtd polynucleotide same in an amount effective to inhibit or stimulate trophoblast cell death, differentiation, invasion, and/or cell fusion and turnover, and an appropriate carrier, diluent, or excipient. A “Mtd polypeptide” includes without limitation Mtd-P, Mtd-L and Mtd-S; and “Mtd polynucleotide” includes without limitation a polynucleotide encoding a Mtd polypeptide.

In an aspect of the invention, a composition is provided for treating a woman suffering from, or who may be susceptible to preeclampsia, comprising a therapeutically effective amount of an inhibitor of a Mtd polypeptide (e.g. Mtd-L and/or Mtd-P), and/or a polynucleotide encoding same and a carrier, diluent, or excipient. In another embodiment of the invention, a composition is provided for monitoring or treating choriocarcinoma or hydatiform mole in a subject comprising a therapeutically effective amount of a Mtd polypeptide or polynucleotide encoding same or a stimulator of same, and a carrier, diluent, or excipient.

The invention provides methods of treatment using the polypeptides, antibodies, polynucleotides, substances and compounds of the invention. In an aspect, the present invention relates to a method for detecting, preventing, and/or treating a condition requiring regulation of trophoblast cell death, differentiation, invasion, and/or cell fusion and turnover by modulating a Mtd polypeptide in particular a Mtd-P Related Polypeptide, or a polynucleotide encoding a Mtd polypeptide, in particular a Mtd-P Related Polypeptide. In an embodiment, a method for treating cancer is provided comprising administering to a patient in need thereof, a Mtd-P Related Polypeptide of the invention, a substance or compound identified using the methods of the invention, or a composition of the invention. In another embodiment, a method for treating preeclampsia is provided comprising administering to a patient in need thereof an inhibitor of a Mtd-P Related Polypeptide or a Mtd Related Polynucleotide (e.g. antisense, micRNA molecule, a substance or compound identified using the methods of the invention, or a composition of the invention).

The invention also contemplates a method for regulating trophoblast cell death, differentiation, invasion, and/or cell fusion and turnover comprising inhibiting or stimulating a Mtd polypeptide or a polynucleotide encoding a Mtd polypeptide.

In an embodiment of the invention, a method is provided for reducing trophoblast cell death, differentiation, invasion, and/or cell fusion and turnover in a subject comprising administering to the subject an effective amount of a modulator (e.g. inhibitor) of a Mtd polypeptide or a polynucleotide encoding a Mtd polypeptide. In a preferred embodiment of the invention a method is provided for treating a woman suffering from, or who may be susceptible to preeclampsia comprising administering therapeutically effective dosages of an inhibitor of a Mtd polypeptide (e.g. Mtd-L and/or Mtd-P). A therapeutically effective dosage is an amount of an inhibitor of effective to down regulate or inhibit a Mtd polypeptide or a polynucleotide encoding the Mtd polypeptide in the woman.

In another embodiment of the invention, a method is providing for reducing trophoblast cell death, differentiation, invasion, and/or cell fusion and turnover in a subject comprising administering an effective amount of a Mtd polypeptide or a polynucleotide encoding the polypeptide, or a stimulator of same. In a preferred embodiment, a method is provided for monitoring or treating choriocarcinoma or hydatiform mole in a subject comprising administering therapeutically effective dosages of a Mtd polypeptide (e.g., Mtd-P) or a stimulator of same. An amount is administered which is effective to up regulate or stimulate a Mtd polypeptide or polynucleotide encoding same in the subject.

A method of the invention may in the alternative or additionally comprise inhibiting or stimulating other polypeptides associated with regulating trophoblast cell death, differentiation, invasion, and/or cell fusion and turnover [e.g., Mcl-1 isoforms (in particular Mcl-1S or Mcl-1L, or caspase cleaved Mcl-1S or Mcl-1L, in particular caspase cleaved Mcl-1L), TGFβ3, HIF1α, PHD1, PHD2, PHD3, VHL, Siah1/2, cullin 2, NEDD8, VEGF, FIH, syncytin, cleaved caspase (e.g., caspase-3), Fas, and/or p53].

In an aspect of the invention a method is provided for diagnosing in a subject a condition requiring modulation of or involving trophoblast cell death, differentiation, invasion, and/or cell fusion and turnover, comprising detecting a Mtd polypeptide (e.g. Mtd-L, Mtd-S and/or Mtd-P) or a polynucleotide encoding a Mtd polypeptide in a sample from the subject. In an embodiment of the diagnostic method of the invention, a method is provided for diagnosing increased risk of preeclampsia in a subject comprising detecting a Mtd polypeptide (Mtd-L and/or Mtd-P) or a polynucleotide encoding a Mtd-P Polypeptide in a sample from the subject.

A diagnostic method of the invention may optionally comprise detecting other markers associated with trophoblast cell death, differentiation, invasion, and/or cell fusion and turnover, or hypoxia [e.g., myeloid cell leukemia factor-1 (Mcl-1) isoforms or caspase cleaved Mcl-1 isoforms, transforming growth factor β3 (TGFβ3) (Caniggia, I, et al, J Clin Invest. 1999 June; 103(12):1641-50, hypoxia inducible transcription factors-1alpha and -2alpha (HIF-1α, HIF-2α) (Caniggia, I. et al. Placenta. 2000 March-April; 21 Suppl A:S25-30 Wang, G L, et al, Proc Natl Acad Sci USA 1995: 92:5510-4; Biol Reprod 2001; 64:499-506; Biol Reprod 2001; 64:1019-1020), prolyl hydroxylating domain-containing 1 (PHD1), prolyl hydroxylating domain-containing 2 (PHD2), prolyl hydroxylating domain-containing 3 (Epstein, A C, et al, Cell 2001; 107:43-54), E3 ligases Siah1/2 (Nakayama, K. and Z. Ronai, Cell Cycle 3:11, 1345-1347), cullin 2, neural precursor cell expressed, developmentally down-regulated 8 NEDD8), syncytin, Fas, VEGF, FIH, cleaved caspase (e.g., caspase-3), and/or p53].

The invention also relates to kits for carrying out the methods of the invention.

These and other aspects, features, and advantages of the present invention should be apparent to those skilled in the art from the following drawings and detailed description.

### DESCRIPTION OF THE DRAWINGS

The invention will now be described in relation to the drawings in which:

FIG. 1. Mtd Expression in First-trimester Placental Tissues. a: RT-PCR followed by southern hybridization to a ³²P-labeled full-length Mtd cDNA in trimester placental tissues. β-actin shown as control. b: Representative Mtd Western blot of first-trimester tissues. Ponceau staining depicts protein loading. c: Fold change in the transcript level of Mtd-L and Mtd-P in early 1st trimester samples (6-8 weeks, black bars, n=14) vs. later gestations (10-12 weeks, open bars, n=10) assessed by quantitative real-time PCR. d: Fold change in protein level of Mtd-L, Mtd-S and Mtd-P in early 1st trimester samples (6-8 weeks, black bars, n=11) vs. later gestations (10-12 weeks, open bars, n=6) assessed by densitometry. e: Spatial localization of Mtd in first-trimester tissue sections. Immunopositivity is represented by brownish staining. Lower panels show TUNEL staining (greenish fluorescence) in neighboring Mtd-stained sections. Middle panels show controls (no 1° antibody). (CT: cytotrophoblast; S: stroma and ST: syncytiotrophoblast). *P<0.05, Student's t test.

FIG. 2. Genomic and mRNA Maps of Human Mtd Isoforms. a: Human chromosomal structure and transcript maps of Mtd isoforms. Exons (I-V) are shown in colour. Conserved BH domains are shown as black boxes. b: Protein sequence alignment between Mtd-L and Mtd-P. Exon II deletion is depicted by a dashed line.

FIG. 3. Mtd Expression in Normal and Preeclamptic Placentae. a: Representative RT-PCR followed by southern blotting for Mtd in tissues from age-matched control (AMC) and preeclamptic (PE) subjects. β-Actin shown as control. b: Mtd Western blot of control and preeclamptic samples. Ponceau staining demonstrates equal protein loading. c: Fold change in transcript level of Mtd-L and Mtd-P in early onset preeclamptic (25-33 weeks, black bars, n=13) vs. age-matched control tissues (open bars, n=9) assessed by quantitative real-time PCR d: Mtd-L, S and P protein densitometric analysis in early onset preeclampsia (black bars, n=23) versus age-matched control patients (open bars, n=25). e: Representative Mtd immunoblot of early onset preeclamptic tissues (PE), late preeclamptic+IUGR tissues (PE/IUGR), placental tissue from IUGR pregnancies (IUGR), essential hypertension (EH) and normal term patients. f: Mtd immunoblot of late (term) onset preeclamptic tissues and normal term patients. Data presented as mean±SEM of three separate experiments. *P<0.05, Student's t test.

FIG. 4. Mtd Immunolocalization in Normal and Preeclamptic Placentae. Mtd staining in normotensive 25 and 27 weeks age-matched control placentae (top) as well as 25 and 27 weeks preeclamptic placentae (bottom). TUNEL analysis performed in neighboring sections is also depicted. Control slides (no 1° antibody) are shown. (SK: syncytial knots). Immunostaining was performed in 10 different normal and preeclamptic placentae (25 to 34 weeks of gestation).

FIG. 5. Functional Analysis of Mtd-P in CHO and BeWo Cells. a: Empty vector (top) and Mtd-P transfected cells (bottom) (left: CHO and right: BeWo) 24-hours post-transfection. Blue staining (β-gal positivity) identifies transfected cells. Graph: Percent of dead cells of total β-gal-expressing blue cells resulting from Mtd-L and Mtd-P transfections compared to empty vector transfected control CHO and BeWo cells. b: Representative mitochondrial JC-1 staining of mock-(empty vector, top) and Mtd-P transfected CHO cells (bottom). Same cell depicted 4 hours and 12 hours post-transfection in each condition is shown. FACS analysis (lower panels) of JC-1 labeled populations of mock-, Mtd-L and Mtd-P transfected CHO cells (untreated live cells and dead cells (used as positive controls) is represented. Y-axis displays FL2 measurement (red fluorescence: indicative of J-aggregates or high mitochondrial membrane potential) and the X-axis displays FL1 measurement (green fluorescence: indicative of J-monomers or loss of mitochondrial membrane potential). Data presented as mean±SEM of three or more separate experiments. *P<0.05, Student's t test. c: Cleaved caspase-3 Western blot analysis performed on total protein isolated from empty vector, Mtd-L and Mtd-P transfected cells 18-hours post-transfection (top: CHO, bottom: BeWo). Ponceau depicts protein loading. d: Nuclear DNA extracted from empty vector, Mtd-L and Mtd-P transfected cells 18-hours posttransfection (top: CHO, bottom: BeWo)

FIG. 6. Effect of Reduced Oxygenation/Oxidative Stress on Mtd Expression in First-trimester Villous Explant Cultures. Immunohistochemical localization of Mtd (3%: a,c; 20%: b,d) and ssDNA (Control (no 1° Ab): e; 3%: f; 20%: g) in explants treated under varying O₂ tension. Neighboring sections to Mtd (c-d) were also stained with anti-ssDNA (e-f), (higher magnification). Brownish staining represents immunopositivity (EVT: extravillous trophoblast cells). Immunostaining representative of 6 different experiments carried out in triplicate. h: qRT-PCR analysis of explants maintained at 3% and 20% O2 and subjected to hypoxia/re-oxygenation (H/R) showing fold changes in Mtd-L (black bars) and Mtd-P (white bars) expression levels compared to 20% O₂ (5 experiments carried out in triplicate). i: A representative Mtd immunoblot performed on protein lysates obtained from villous explants cultures under 3% and 20% O₂ as well as under conditions of HR. j: Transcript expression levels of Mtd isoforms in explants treated with Mtd isoform-specific antisense (AS-L and P, gray bars) relative to control sense (S-L and P, black bars) as assessed by q-RT-PCR (data are normalized to untreated tissue: C, white bar). k: A representative cleaved caspase-3 immunoblot performed on protein lysates obtained from villous explants under control conditions (C: no oligos) and treated with Mtd-sense (S-L and S-P respectively) and Mtd-antisense (AS-L and AS-P respectively) oligos. All experimental conditions were performed in triplicate in three independent experiments. Ponceau staining demonstrates protein loading. *P<0.05, Student's t test.

FIG. 7. Putative Model: Mtd Role in Normal and Preeclamptic Placentae. In preeclampsia, alteration in placental oxygenation, due to low pO₂ or oxidative stress (HR) may be responsible for the aberrant expression of Mtd-P, which in turn leads to a change in the physiological apoptotic rheostat in trophoblast cells. This imbalance results in accelerated syncytiotrophoblast cell death resulting in increased shedding of trophoblast microfragments in the maternal circulation.

FIG. 8. Human Genomic, transcript and protein maps of Mcl-1 isoforms. Mcl-1 is located on chromosome 1q21 and comprises three exons all of which encode for protein sequence. Alternate splicing gives rise to distinct Mcl-1 mRNAs either containing or lacking exon 2 and encoding respectively the Mcl-1L and Mcl-1S which lacks the TM domain (ATM). The structure and size (in amino-acid residues) of the Mcl-1L and Mcl-1S isoforms are depicted. The PEST (proline (P); glutamic acid (E); serine (S); or threonine (T)), the BH (Bcl-2 homology) and the TM (transmembrane) domains are indicated, along with the caspase-3 cleavage sites at Asp127 and Asp157. Mcl-1S, which also contains these residues, is also subjected to caspase-mediated cleavage. Skipping of exon II in Mcl-1S, shifts the reading frame of the full-length protein, resulting in loss of the BH1, BH2 and transmembrane domains, hence generating a truncated pro-apoptotic “BH3-only” containing isoform. Finally, one of the Mcl-1 cleavage products generated by caspase-mediated cleavage within the PEST domain is also depicted. (Michels et al., Int J Biochem Cell Biol. 2005 February; 37(2):267-71)

FIG. 9. Mcl-1 Expression in Placentae of Patients Diagnosed with Severe Early Onset Preeclampsia. a: RT-PCR followed by Southern blotting with a full-length Mcl-1L 32P-labeled probe in placental tissues from severe early-onset preeclampsia and normotensive age-matched control tissues. b-actin shown as control. b: Quantitative real-time RT-PCR analysis of Mcl-1L and Mcl-1S transcripts in placental tissues from severe early-onset preeclampsia (PE: Black bar) and normotensive age-matched control tissues (AMC: Open bar). c: Representative Mcl-1 immunoblot performed on AMC and PE total protein lysates (Mcl-1L: classic long isoform; Mcl-1c (or p28): the predominant Mcl-1L cleaved byproduct and Mcl-1S: short isoform). d: Densitometric analysis of Mcl-1-specific protein isoform bands between AMC (open bar; n=22) and PE (black bar; n=25). All immunoblots were confirmed for equal protein loading using ponceau staining. Data are presented as mean±SE of three or more separate experiments. *P<0.05, Student's t test.

FIG. 10. Mcl-1 Expression in Term Preeclamptic Placentae and Other Subpathologies. a: Representative Mcl-1 immunoblot performed on total protein lysates from normal term placentae (term), term preeclamptic placentae (term PE) and normal term elective caesarian section placentae in absence of labour (C/S). b: Representative Mcl-1 immunoblot performed on total protein lysates from placentae from severe early-onset preeclampsia, IUGR pregnancies, 35-37 weeks IUGR+PE pregnancies, pregnant patients with essential hypertension (EH) and normal term placentae (term). All immunoblots were confirmed for equal protein loading using ponceau staining. Data are presented as mean±SE of three or more separate experiments. *P<0.05, Student's t test.

FIG. 11. Effect of Varying Oxygenation on Mcl-1 expression. a: Quantitative RT-PCR analyses of Mcl-1 isoforms L (black box) and S (white box) in first trimester villous explants exposed to 20% O₂,3% O₂ and hypoxia/reoxygenation (H/R) conditions. b: Representative immunoblot of Mcl-1 isoforms in first trimester villous explants exposed to 20% O₂,3% O₂ and H/R. c: Representative immunoblot of Mcl-1 isoforms in control (untreated explant) and explants exposed to H/R in presence of 100 mM concentration of pan-caspase inhibitor z-VAD-fink dissolved in DMSO relative to control-treatment (DMSO alone). d: Representative Mcl-1 immunoblot performed on protein lysates from explants exposed to H/R in presence of 100 mM z-DEVD-fmk (in DMSO) and absence of caspase-3 inhibitor (DMSO alone) relative to untreated control tissue (Control). All immunoblots were confirmed for equal protein loading using ponceau staining. Data are presented as mean±SE of three or more separate experiments. *P<0.05, Student's t test.

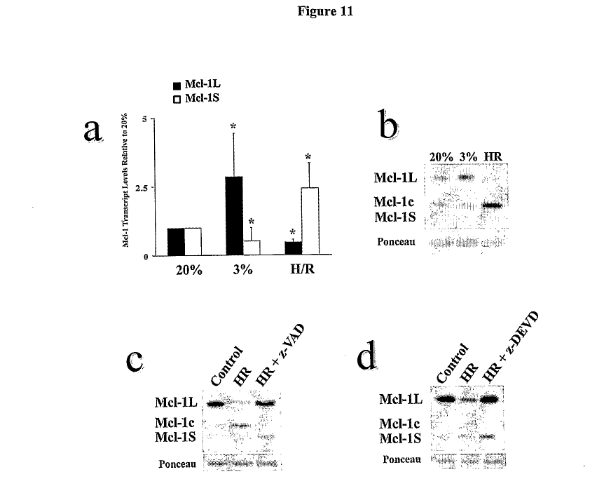

FIG. 12. Transcript Expression of Mcl-1 and Mtd Isoforms in Placental Tissue from SL, MA and HA Pregnancies. A, B, C and D: Respectively, quantitative RT-PCR analysis of Mtd-L (pro-apoptotic), Mtd-P (pro-apoptotic), Mcl-1L (anti-apoptotic) and Mcl-1S (pro-apoptotic) transcript expression in sea-level (SL), moderate altitude (MA) and high altitude (HA) placentae. These data suggest a shift of Mtd-Mcl-1 transcripts towards protective isoforms under conditions of chronic placental hypoxia. Data are presented as mean±SE. *P<0.05, Student's t test.

FIG. 13. Protein Expression of Mcl-1 and Mtd Isoforms in Placental Tissue Tissue from SL, MA and HA Pregnancies. A and B: Respectively, Mcl-1 and Mtd immunoblots of protein lysates obtained from Sea Level (SL), Moderate Altitude (MA), High Altitude (HA) placentae. Mtd expression is unchanged between the various altitudes whereas Mcl-1L expression is increased in HA relative to lower altitudes. Equal protein loading was checked by ponceau staining. Data are presented as mean±SE. *P<0.05, Student's t test. C: Representative immunoblot of cleaved caspase-3 performed on total protein lysates obtained from HA, MA and SL placental tissues demonstrating reduced caspase-3 cleavage under conditions of chronic placental hypoxia. D: Immunohistochemical localization of Mcl-1 (Top panels) and Mtd (Lower panels) in SL, MA and HA placentae. (S: stroma, ST: syncytium). Mtd staining is unaffected by altitude where as Mcl-1 staining increases in HA versus lower altitudes. Both Mtd and Mcl-1 localize predominately to trophoblast cell layers. Staining representative of 4 separate experiments carried out in triplicate.

FIG. 14. Effect of Varying Oxygen Tension on the Expression of Mcl-1 Transcript and Protein isoforms in Villous Explants. A: Quantitative RT-PCR analyses of Mcl-1 isoforms L (black box) and S (white box) in first trimester villous explants exposed to 3% O₂ vs. 20% O₂. B: Representative immunoblot of Mcl-1 protein isoforms L (43 kDa) and S (33 kDa) in first trimester villous explants exposed to 3% O₂ vs 20% O₂. Mcl-1 protein and transcript expression increased and decreased with respect to L and S isoforms respectively under reduced oxygenation relative to standard conditions. Data are presented as mean±SE of three or more separate experiments. *P<0.05, Student's t test.

FIG. 15. Syncytin Transcript Expression in SL, MA and HA placentae. Quantitative RT-PCR analysis of syncytin transcript in Sea Level (SL), Moderate Altitude (MA), and High Altitude (HA) placental tissues. Syncytin expression significantly decreases in placentae from HA relative to MA and SL. Data are presented as mean±SE of three separate experiments. *P<0.05, Student's t test.

FIG. 16. Altered Trophoblast Cell Death and Differentiation in HA vs Lower Altitude placentae. In pregnancies from altitude-induced chronic hypoxia a shift in Mcl-1/Mtd rheostat favors trophoblast cell survival. This may slow-down trophoblast cell turnover and death as demonstrated by a decreased level of syncytin expression.

FIG. 17 are graphs and an immunoblot showing the expression of VHL in normal and preeclamptic placentae.

FIG. 18 are graphs showing the results of qRT-PCR analysis of PHDs in placental tissue from normal and preeclamptic pregnancies.

FIG. 19 are graphs showing the results of qRT-PCR analysis of PHDs in placental tissue from normal and severe IUGR pregnancies.

FIG. 20 are graphs and immunoblots showing the relative expression of SIAH1, SIAH2, PHD1, PHD2, and PHD3 in tissue from normal and severe IUGR pregnancies.

FIG. 21 are graphs showing the protein content of HIF-1α, HIF-1β, VHL and NEDD8/CUL2 in placental tissue from normal and preeclamptic pregnancies.

FIG. 22 are graphs and an immunoblot showing the expression levels of FIH and VEGF in tissue from normal and severe IUGR pregnancies.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

### 1. Polynucleotides

As hereinbefore mentioned, the invention provides an isolated polynucleotide having a sequence encoding a Mtd-P. The term “isolated” refers to a nucleic acid substantially free of cellular material or culture medium when produced by recombinant DNA techniques, or chemical reactants, or other chemicals when chemically synthesized. An “isolated” nucleic acid may also be free of sequences which naturally flank the nucleic acid (i.e., sequences located at the 5′ and 3′ ends of the polynucleotide) from which the nucleic acid is derived. The term “nucleic acid” is intended to include DNA and RNA and can be either double stranded or single stranded. In an aspect, a polynucleotide of the invention encodes a polypeptide comprising an amino acid sequence of SEQ ID NO: 1, preferably a polynucleotide of the invention comprises a nucleic acid sequence of SEQ ID NO: 2.

In an embodiment, the invention provides an isolated polynucleotide which comprises:
(i) a nucleic acid sequence encoding a polypeptide having substantial sequence identity with an amino acid sequence of SEQ ID NO: 1;
(ii) a nucleic acid sequence encoding a polypeptide comprising an amino acid sequence of SEQ ID NO: 1;
(iii) nucleic acid sequences complementary to (i) or (ii);
(iv) a degenerate form of a nucleic acid sequence of (i) or (ii);
(v) a nucleic acid sequence capable of hybridizing under stringent conditions to a nucleic acid sequence in (i), (ii) or (iii);
(vi) a nucleic acid sequence encoding a truncation, an analog, an allelic or species variation of a polypeptide comprising an amino acid sequence of SEQ ID NO: 1; or
(vii) a fragment or allelic or species variation of (i), (ii) or (iii).

Preferably, a purified and isolated polynucleotide of the invention comprises:
(i) a nucleic acid sequence comprising the sequence of SEQ ID NO: 2, wherein U can also be T;
(ii) nucleic acid sequences complementary to (i), preferably complementary to the full nucleic acid sequence of SEQ ID NO: 2;
(iii) a nucleic acid capable of hybridizing under stringent conditions to a nucleic acid of (i) or (ii) and preferably having at least 18 nucleotides; or
(iv) a polynucleotide differing from any of the nucleic acids of (i) to (iii) in codon sequences due to the degeneracy of the genetic code.

The invention includes nucleic acid sequences complementary to a nucleic acid encoding a polypeptide comprising an amino acid sequence of SEQ ID NO: 1.

The invention includes polynucleotides having substantial sequence identity or homology to nucleic acid sequences of the invention or encoding polypeptides having substantial identity or similarity to the amino acid sequence of SEQ ID NO: 1. Preferably, the nucleic acids have substantial sequence identity for example at least 80% or 85% nucleic acid identity; more preferably 90% nucleic acid identity; and most preferably at least 95%, 96%, 97%, 98%, or 99% sequence identity.

“Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part I, Griffin, A. M. and Griffin, H. G. eds., Humana Press, New Jersey, 1994; Sequence Analysis in Molecular Biology, von Heinje, G. Academic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Isolated polynucleotides encoding Mtd-P Related Polypeptides and having a sequence which differs from a nucleic acid sequence of the invention due to degeneracy in the genetic code are also within the scope of the invention. Such nucleic acids encode functionally equivalent polypeptides (e.g., a Mtd-P Related Polypeptide) but differ in sequence from the sequence of a Mtd-P Polypeptide due to degeneracy in the genetic code. As one example, DNA sequence polymorphisms within the nucleotide sequence of a Mtd-P Polynucleotide may result in silent mutations which do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of a Mtd-P Polypeptide. These amino acid polymorphisms are also within the scope of the present invention.

Another aspect of the invention provides a polynucleotide which hybridizes under stringent conditions, preferably high stringency conditions to a polynucleotide which comprises a sequence which encodes a Mtd-P Polypeptide having an amino acid sequence shown in SEQ ID NO: 1. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

It will be appreciated that the invention includes polynucleotides encoding a Mtd-P Related Polypeptide including truncations of a Mtd-P Polypeptide, and analogs of a Mtd-P Polypeptide as described herein.

An isolated polynucleotide of the invention which comprises DNA can be isolated by preparing a labelled nucleic acid probe based on all or part of a nucleic acid sequence of the invention. The labelled nucleic acid probe is used to screen an appropriate DNA library (e.g. a cDNA or genomic DNA library). For example, a cDNA library can be used to isolate a cDNA encoding a Mtd-P Related Polypeptide by screening the library with the labelled probe using standard techniques. Alternatively, a genomic DNA library can be similarly screened to isolate a genomic clone encompassing a gene encoding a Mtd-P Related Polypeptide. Nucleic acids isolated by screening of a cDNA or genomic DNA library can be sequenced by standard techniques.

An isolated polynucleotide of the invention which is DNA can also be isolated by selectively amplifying a nucleic acid encoding a Mtd-P Related Polypeptide using the polymerase chain reaction (PCR) methods and cDNA or genomic DNA. It is possible to design synthetic oligonucleotide primers from the nucleotide sequence of the invention for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated polynucleotide of the invention which is RNA can be isolated by cloning a cDNA encoding a Mtd-P Related Polypeptide into an appropriate vector which allows for transcription of the cDNA to produce an RNA molecule which encodes a Mtd-P Related Polypeptide. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Polynucleotides of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular polynucleotide encodes a Mtd-P Related Polypeptide can be accomplished by expressing the cDNA in an appropriate host cell by standard techniques, and testing the expressed protein in the methods described herein. A cDNA encoding a Mtd-P Related Polypeptide can be sequenced by standard techniques, such as dideoxynucleotide chain termination or Maxam-Gilbert chemical sequencing, to determine the nucleic acid sequence and the predicted amino acid sequence of the encoded polypeptide.

The initiation codon and untranslated sequences of a Mtd-P Related Polypeptide may be determined using computer software designed for the purpose, such as PC/Gene (IntelliGenetics Inc., Calif.). The intron-exon structure and the transcription regulatory sequences of a gene encoding a Mtd-P Related Polypeptide may be confirmed by using a polynucleotide of the invention encoding a Mtd-P Related Polypeptide to probe a genomic DNA clone library. Regulatory elements can be identified using standard techniques. The function of the elements can be confirmed by using these elements to express a reporter gene such as the lacZ gene that is operatively linked to the elements. These constructs may be introduced into cultured cells using conventional procedures or into non-human transgenic animal models. In addition to identifying regulatory elements in DNA, such constructs may also be used to identify nuclear proteins interacting with the elements, using techniques known in the art.

In a particular embodiment of the invention, the polynucleotides isolated using the methods described herein are mutant Mtd-P gene alleles. The mutant alleles may be isolated from individuals either known or proposed to have a genotype which contributes to the symptoms of a disorder involving a Mtd-P Related Polypeptide. Mutant alleles and mutant allele products may be used in therapeutic and diagnostic methods described herein. For example, a cDNA of a mutant Mtd gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal Mtd gene or any suitable fragment thereof, may then be labelled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant Mtd allele. Gene products made by the putatively mutant tissue may be expressed and screened, for example using antibodies specific for a Mtd-P Related Polypeptide as described herein. Library clones identified using the antibodies can be purified and subjected to sequence analysis.

The invention contemplates variants of a Mtd-P Polynucleotide. In an aspect, the invention provides a variant of a nucleic acid sequence of SEQ ID NO:2 wherein the nucleic acid sequence encodes a domain having the ability to interact with an anti-apoptotic molecule, and wherein the variant comprises an isolated nucleic acid sequence having at least one mutation resulting in loss of the ability of the domain to interact with the anti-apoptotic molecule. In another aspect, a variant of a nucleic acid sequence of SEQ ID NO:2 is provided wherein the nucleic acid sequence comprises a second exon encoding part of a domain having the ability to interact with an anti-apoptotic molecule, and wherein the variant is selected from the class comprising:
(a) isolated nucleic acid sequences lacking the second exon,
(b) isolated nucleic acid sequences having at least one mutation in the second exon resulting in loss of the ability of the domain to interact with the anti-apoptotic molecule, and
(c) isolated nucleic acid sequences lacking splice sites defining the second exon.

The sequence of a polynucleotide of the invention, or a fragment of the molecule, may be inverted relative to its normal presentation for transcription to produce an antisense polynucleotide. An antisense polynucleotide may be constructed using chemical synthesis and enzymatic ligation reactions using procedures known in the art.

### 2. Polypeptides

An amino acid sequence of a Mtd-P Polypeptide can comprise a sequence as shown in SEQ ID NO: 1. In addition to polypeptides comprising an amino acid sequence as shown in SEQ ID NO: 1, the polypeptides of the present invention include truncations, analogs, and polypeptides having sequence identity or similarity to Mtd-P Polypeptides, and truncations thereof as described herein (i.e., Mtd-P Related Polypeptides).

Aspects of the invention include analogs of a Mtd-P Polypeptide, and/or truncations thereof, which may include, but are not limited to a Mtd-P Polypeptide, containing one or more amino acid substitutions, insertions, and/or deletions. Amino acid substitutions may be of a conserved or non-conserved nature. Conserved amino acid substitutions involve replacing one or more amino acids of a Mtd-P Polypeptide amino acid sequence with amino acids of similar charge, size, and/or hydrophobicity characteristics. When only conserved substitutions are made the resulting analog is preferably functionally equivalent to a Mtd-P Polypeptide. Non-conserved substitutions involve replacing one or more amino acids of the Mtd-P Polypeptide amino acid sequence with one or more amino acids that possess dissimilar charge, size, and/or hydrophobicity characteristics. One or more amino acid insertions may be introduced into a Mtd-P Polypeptide. Amino acid insertions may consist of single amino acid residues or sequential amino acids ranging from 2 to 15 amino acids in length. Deletions may consist of the removal of one or more amino acids, or discrete portions from the Mtd-P Polypeptide sequence. The deleted amino acids may or may not be contiguous. The lower limit length of the resulting analog with a deletion mutation is about 10 amino acids, preferably 20 to 40 amino acids.

The polypeptides of the invention include polypeptides with sequence identity or similarity to a Mtd-P Polypeptide and/or truncations thereof as described herein. Such a Mtd-P Polypeptide include polypeptides whose amino acid sequences are comprised of the amino acid sequences of Mtd-P Polypeptide regions from other species that hybridize under selected hybridization conditions (see discussion of stringent hybridization conditions herein) with a probe used to obtain a Mtd-P Polypeptide. These polypeptides will generally have the same regions which are characteristic of a Mtd-P Polypeptide. Preferably a polypeptide will have substantial sequence identity for example, about 55%, 60%, 65%, 70%, 75%, 80%, or 85% identity, preferably 90% identity, more preferably at least 95%, 96%, 97%, 98%, or 99% identity, and most preferably 98% or 99% identity with an amino acid sequence of SEQ. ID. NO. 1. A percent amino acid sequence homology, similarity or identity, is calculated as the percentage of aligned amino acids that match the reference sequence using known methods as described herein.

The invention also contemplates isoforms of the polypeptides of the invention. An isoform contains the same number and kinds of amino acids as a polypeptide of the invention, but the isoform has a different molecular structure. Isoforms contemplated by the present invention preferably have the same properties as a polypeptide of the invention as described herein.

The present invention also includes Mtd-P Related Polypeptides conjugated with a selected protein, or a marker protein (see below) to produce fusion proteins. Additionally, immunogenic portions of Mtd-P and a Mtd-P Related Polypeptide are within the scope of the invention.

A Mtd-P Related Polypeptide of the invention may be prepared using recombinant DNA methods. Accordingly, the polynucleotides of the present invention having a sequence which encodes a Mtd-P Related Polypeptide of the invention may be incorporated in a known manner into an appropriate expression vector which ensures good expression of the polypeptide. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a recombinant expression vector comprising a polynucleotide of the invention, and the necessary regulatory sequences for the transcription and/or translation of the inserted nucleic acid sequence. Suitable regulatory sequences may be derived from a variety of sources, including bacterial, fungal, viral, mammalian, or insect genes [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen as discussed below, and may be readily accomplished by one of ordinary skill in the art.

The invention further provides a recombinant expression vector comprising a DNA polynucleotide of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of a polypeptide of the invention or a fragment thereof. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin, preferably IgG. The markers can be introduced on a separate vector from the nucleic acid of interest.

The recombinant expression vectors may also contain genes that encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pOEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The recombinant expression vectors may be introduced into host cells to produce a transformant host cell. “Transformant host cells” include host cells which have been transformed or transfected with a recombinant expression vector of the invention. The terms “transformed with”, “transfected with”, “transformation” and “transfection” encompass the introduction of a nucleic acid (e.g., a vector) into a cell by one of many standard techniques. Prokaryotic cells can be transformed with a nucleic acid by, for example, electroporation or calcium-chloride mediated transformation. A nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the polypeptides of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g., cleaves) the protein in a desired fashion. Host systems or cell lines may be selected which have specific and characteristic mechanisms for post-translational processing and modification of proteins. For example, eukaryotic host cells including CHO, VERO, BHK, HeLA, COS, MDCK, 293, 3T3, and WI38 may be used. For long-term high-yield stable expression of the polypeptide, cell lines and host systems which stably express the gene product may be engineered.

Host cells and in particular cell lines produced using the methods described herein may be particularly useful in screening and evaluating compounds that modulate the activity of a Mtd-P Related Polypeptide.

The polypeptides of the invention may also be expressed in non-human transgenic animals including but not limited to mice, rats, rabbits, guinea pigs, micro-pigs, goats, sheep, pigs, non-human primates (e.g. baboons, monkeys, and chimpanzees) [see Hammer et al. (Nature 315:680-683, 1985), Palmiter et al. (Science 222:809-814, 1983), Brinster et al. (Proc Natl. Acad. Sci. USA 82:44384442, 1985), Palmiter and Brinster (Cell. 41:343-345, 1985) and U.S. Pat. No. 4,736,866)]. Procedures known in the art may be used to introduce a polynucleotide of the invention encoding a Mtd-P Related Polypeptide into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the Mtd-P gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant Mtd-P Related Polypeptide in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against Mtd-P Polypeptides.

Polypeptides of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a Mtd-P Related Polypeptide of the invention conjugated with other molecules, such as proteins, may be prepared by fusing, through recombinant techniques, the N-terminal or C-terminal of a Mtd-P Related Polypeptide, and the sequence of a selected protein or marker protein with a desired biological function. The resultant fusion proteins contain a Mtd-P Polypeptide fused to the selected protein or marker protein as described herein. Examples of proteins which may be used to prepare fusion proteins include immunoglobulins, glutathione-S-transferase (GST), hemagglutinin (HA), and truncated myc.

### 3. Antibodies

Mtd-P Related Polypeptides of the invention can be used to prepare antibodies specific for the polypeptides. Antibodies can be prepared which bind a distinct epitope in an unconserved region of the polypeptide. An unconserved region of the polypeptide is one that does not have substantial sequence homology to other polypeptides. A region from a conserved region such as a well-characterized domain can also be used to prepare an antibody to a conserved region of a Mtd-P Related Polypeptide. Antibodies having specificity for a Mtd-P Related Polypeptide may also be raised from fusion proteins created by expressing fusion proteins in bacteria as described herein.

The invention can employ intact monoclonal or polyclonal antibodies, and immunologically active fragments (e.g. a Fab, (Fab)₂ fragment, or Fab expression library fragments and epitope-binding fragments thereof), an antibody heavy chain, an antibody light chain, a genetically engineered single chain Fv molecule (Ladner et al, U.S. Pat. No. 4,946,778), humanized antibody, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

### 4. Applications

The polynucleotides, Mtd-P Related Polypeptides, and antibodies of the invention may be used in the prognostic and diagnostic evaluation of disorders involving a Mtd-P Related Polypeptide or conditions requiring regulation of trophoblast cell death, differentiation, invasion, and/or cell fusion and turnover (e.g., preeclampsia), and the identification of subjects with a predisposition to such disorders or conditions.

Methods for detecting polynucleotides and Mtd-P Related Polypeptides can be used to monitor disorders involving a Mtd-P Related Polypeptide or conditions requiring regulation of trophoblast cell death, differentiation, invasion, and/or cell fusion and turnover, by detecting the Mtd-P transcripts, Mtd-P Related Polypeptides and polynucleotides encoding Mtd-P Related Polypeptides. The applications of the present invention also include methods for the identification of compounds that modulate the biological activity of Mtd-P Related Polypeptides (Section 4.4). The compounds, antibodies, etc., may be used for the treatment of disorders involving a Mtd-P Related Polypeptide or Mtd-P Related Polynucleotides (Section 4.5). It would also be apparent to one skilled in the art that the methods described herein may be used to study the developmental expression of Mtd-P Related Polypeptides and, accordingly, will provide further insight into the role of Mtd-P Related Polypeptides.

### 4.1 Diagnostic Methods

A variety of methods can be employed for the detection, diagnosis, monitoring, and prognosis of conditions described herein, or status of conditions described herein, and for the identification of subjects with a predisposition to such conditions. Such methods may, for example, utilize Mtd-P Related Polynucleotides, and fragments thereof, and binding agents (e.g. antibodies) against one or more Mtd-P Related Polypeptide, including peptide fragments. In particular, polynucleotides and antibodies may be used, for example, for (1) the detection of the presence of Mtd-P Related Polynucleotide mutations, or the detection of either an over- or under-expression of Mtd-P Related Polynucleotide mRNA relative to a normal state, or the qualitative or quantitative detection of alternatively spliced forms of Mtd-P Related Polynucleotide transcripts which may correlate with certain conditions or susceptibility toward a condition; and (2) the detection of either an over- or an under-abundance of one or more Mtd-P Related Polypeptides relative to a normal state or a different stage of a condition, or the presence of a modified Mtd-P Polypeptide which correlates with a condition or state, or a progression toward a condition, or a particular type or stage of a condition.

The methods described herein can be adapted for diagnosing and monitoring a condition involving a Mtd-P Related Polypeptide and/or a Mtd-P Related Polynucleotide by detecting one or more Mtd-P Related Polypeptide or Mtd-P Related Polynucleotides in biological samples from a subject. These applications require that the amount of Mtd-P Related Polypeptide or Mtd-P Related Polynucleotides quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of condition, may be established by prospective and/or retrospective statistical studies. Healthy subjects who have no clinical evidence of a condition or abnormalities may be selected for statistical studies. Diagnosis may be made by a finding of altered levels, in particular statistically different levels of detected Mtd-P Related Polypeptide or Mtd-P Related Polynucleotides associated with a condition (e.g., preeclampsia) compared to a control sample or previous levels quantitated for the same subject. “Statistically different levels”, or “significant difference” in levels of markers in a patient sample compared to a control or standard may represent levels that are higher or lower than the standard error of the detection assay. In particular embodiments, the levels may be 1.5, 2, 3, 4, 5, or 6 times higher or lower than the control or standard.

In an aspect of the invention, a method is provided for diagnosing or monitoring in a subject a condition mediated by a Mtd-P Related Polypeptide, in particular a condition requiring modulation of or involving trophoblast cell death, differentiation, invasion, and/or cell fusion and turnover (e.g., preeclampsia), comprising detecting a Mtd-P Related Polypeptide and/or Mtd-P Related Polynucleotide in a sample from the subject. In an embodiment of a diagnostic method of the invention, a method is provided for diagnosing increased risk of a condition requiring modulation of or involving trophoblast cell death, differentiation, invasion, and/or cell fusion and turnover (e.g., preeclampsia) in a subject comprising detecting a Mtd-P Related Polypeptide and/or Mtd-P Related Polynucleotide in a sample from the subject.

In another aspect, the invention provides use of binding agents or polynucleotides that interact with a Mtd polypeptide and optionally other polypeptide markers disclosed herein, or with a polynucleotide encoding a Mtd polypeptide and optionally other polynucleotide markers disclosed herein, in the manufacture of a composition for detecting a condition requiring modulation of or involving trophoblast cell death, differentiation, invasion, and/or cell fusion and turnover (e.g., preeclampsia, choriocarcinoma, hydatiform mole, or molar pregnancy).

The methods described herein may be used to predict or evaluate the probability of a condition disclosed herein (e.g., preeclampsia), for example, in a sample freshly removed from a host. Such methods can be used to detect the condition (e.g., preeclampsia) and help in the diagnosis and prognosis of the condition. The methods can be used to detect the potential for the condition and to monitor a patient or a therapy.

The invention also contemplates a method for detecting a condition disclosed herein (e.g. a condition requiring modulation of or involving trophoblast cell death, differentiation, invasion, and/or cell fusion and turnover), or a predisposition to such condition, comprising producing a profile of levels of one or more Mtd-P Related Polypeptide and/or Mtd-P Related Polynucleotide in a sample (e.g. cells) from a patient, and comparing the profile with a reference to identify a profile for the patient indicative of the condition.

The methods described herein may also use multiple markers for a condition described herein, in particular preeclampsia. Therefore, the invention contemplates a method for analyzing a biological sample for the presence of one or more Mtd-P Related Polypeptide and/or Mtd-P Related Polynucleotide, and other markers that are specific indicators of the condition. The methods described herein may be modified by including reagents to detect the additional markers. In particular, the methods may optionally comprise producing a profile of levels of other markers associated with the condition. For example, a method of the invention for diagnosing preeclampsia may also comprised detecting or producing profiles of levels of Mtd-P (SEQ ID NO. 1), Mtd-L (SEQ ID NO 3), Mtd-S, transforming growth factor β3 (TGFβ3) (e.g., Accession No. NP_{—}003230); transforming growth factor β1 (TGFβ1) (e.g., Accession No. NP_{—}000651); hypoxia-inducible factor 1, alpha subunit (HIF-1α) (e.g., Accession No. NP_{—}001521); hypoxia-inducible factor 1, beta subunit (HIF-1β) (e.g., Accession No. NP_{—}001659; NP_{—}848513; NP_{—}848514); hypoxia-inducible factor 2, alpha subunit (HIF-2α) (e.g., Accession No. Q99814); von Hippel-Lindau tumor suppressor (VHL) (e.g., Accession Nos. NP_{—}000542 and NP_{—}937799); myeloid cell leukemia sequence 1 (Mcl-1) (e.g., Accession Nos. NP_{—}068779-isoform 1; NP_{—}877495-isoform 2; SEQ ID NO. 5, 6, or 9); prolyl-4-hydroxylase 1 (PHD1) (e.g., Accession No. NP_{—}071334); prolyl-4-hydroxylase 2 (PHD2) (e.g., Accession No. NP_{—}0600251; NP_{—}542770; and NP_{—}444274); prolyl-4-hydroxylase 3 (PHD3) (e.g., Accession No. NP_{—}071356); seven in absentia homolog 1 (Siah1) (e.g., Accession No. NP_{—}001006611 and NP_{—}003022); seven in absentia homolog 2 (Siah2) (e.g., Accession No. NP_{—}005058); vascular endothelial growth factor (VEGF) (e.g., Accession No. NP_{—}001020537 to NP_{—}001020541, NP_{—}003367); syncytin (e.g., Accession No. NP_{—}055405); cullin 2 (e.g., Accession No. NP_{—}003582); neural precursor cell expressed, developmentally down-regulated 8 (NEDD) (e.g. Acession No. NP_{—}006147); factor inhibiting HIF(FIH) (e.g., Accession No. Q9NWT6); Fas (TNF receptor superfamily, member 6) (e.g., Accession No. NP_{—}000034; NP_{—}690610 through NP_{—}690616); cleaved caspase-3 (e.g., capase-3: Accession No. NP_{—}004337; NP_{—}116786; AAO25654); and tumor protein p53 (e.g., Accession No. NM_{—}000546); or, polynucleotides encoding these polypeptides. Exemplary nucleic acid sequences encoding the polypeptides are as follows: Mtd-P (SEQ ID NO. 2), Mtd-L (SEQ ID NO 4), TGFβ3 (e.g. Accession No. NM_{—}003239, NM_{—}181054); (TGFβ1) (e.g., Accession No. NM_{—}000660); HIF-1α (e.g., Accession No. NM_{—}001530, NP_{—}851397); VHL (e.g. Accession Nos. NM_{—}000551, NM_{—}198156); Mcl-1 (e.g., Accession Nos. NM_{—}021960-variant 1; NM_{—}182763-variant 2; SEQ ID NO. 7, 8, and 10); PHD1 (e.g., Accession No. NM_{—}022051); PHD2 (e.g., Accession Nos. NM_{—}053046, NM_{—}017555, NM_{—}080732); PHD3 (e.g., Accession No. NM_{—}022073); HIF-1β (e.g., Accession No. NM_{—}001668; NM_{—}178426; NM_{—}178427); seven in absentia homolog 1 (Siah1) (e.g., Accession Nos. NM_{—}001006610; NM_{—}0030311 and NM_{—}001006611); Siah2 (e.g., Accession No. NM_{—}005067); VEGF (e.g., Accession No. NM_{—}001025366 to NM_{—}001025370, NM_{—}003376), FIH-1 (e.g. Accession No. AF395830), syncytin (e.g., Accession No. NG_{—}004112), CUL2 (e.g., Accession No. NM_{—}003591); NEDD8 (e.g. Accession No. NM_{—}006156), Fas (e.g., Accession Nos. NM_{—}000043; NM_{—}152871; NM_{—}152872; NM_{—}152873 152877); cleaved caspase-3 (e.g., caspase-3: Accession No. NM_{—}004346; NM_{—}032991; AY219866); and, p53 (e.g., Accession No. NP_{—}000537).

It will be appreciated that the markers disclosed herein include but are not limited to native-sequence polypeptides, isoforms, chimeric polypeptides, and all homologs, fragments, and precursors of the markers, including modified forms of the polypeptides and derivatives.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Such variants include, for instance, polypeptides wherein one or more amino acid residues are added to, or deleted from, the N- or C-terminus of the full-length or mature sequences of the polypeptide, including variants from other species, but excludes a native-sequence polypeptide. In aspects of the invention variants retain the immunogenic activity of the corresponding native-sequence polypeptide. An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis.

A “chimeric protein” or “fusion protein” comprises all or part (preferably biologically active) of a marker operably linked to a heterologous polypeptide (i.e., a polypeptide other than the marker). Within the fusion protein, the term “operably linked” is intended to indicate that a marker and the heterologous polypeptide are fused in-frame to each other. The heterologous polypeptide can be fused to the N-terminus or C-terminus of the marker. A useful fusion protein is a GST fusion protein in which a marker is fused to the C-terminus of GST sequences. Chimeric and fusion proteins can be produced by standard recombinant DNA techniques.

A modified form of a polypeptide referenced herein includes modified forms of the polypeptides and derivatives of the polypeptides, including post-translationally modified forms such as glycosylated, phosphorylated, acetylated, methylated or lapidated forms of the polypeptides.

The markers disclosed herein may be prepared by recombinant or synthetic methods, or isolated from a variety of sources, or by any combination of these and similar techniques.

The invention provides a set of markers correlated with trophoblast cell death, differentiation, invasion, and/or cell fusion or turnover. A set of these markers that can be used for detection, diagnosis, prevention and therapy of conditions disclosed herein includes Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ1, TGFβ3, HIF-1α, HIF-1β, HIF-2 α, VHL, cullin 2, NEDD8, PHD1, PHD2, PHD3, Siah1/2, cleaved caspase (e.g. caspase-3), syncytin, Fas, VEGF, FIH, and/or p53. Thus, the invention provides marker sets that distinguish a condition requiring modulation of trophoblast cell death, differentiation, invasion, and/or cell fusion or turnover and uses therefor. In an aspect, the invention provides a method for classifying a condition requiring modulation of trophoblast cell death, differentiation, invasion, and/or cell fusion or turnover comprising detecting a difference in the expression of a plurality of markers relative to a control, the plurality of markers or polynucleotide markers comprising at least one, two, three, four, five, six, seven, eight, nine, or ten or more of Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, HIF-1α, HIF-1β, HIF-2 α, VHL, cullin 2, NEDD8, PHD1, PHD2, PHD3, Siah1/2, cleaved caspase (e.g. caspase-3), syncytin, Fas, VEGF, FIH, and/or p53, and/or polynucleotides encoding same. In an aspect the marker set comprises Mtd-P and/or Mtd-L, and one or more of Mcl-1 isoforms (in particular Mcl-1S or Mcl-1L, or caspase cleaved Mcl-1S or Mcl-1L, in particular caspase cleaved Mcl-1L), TGFβ1, TGFβ3, HIF-1α, HIF-1β, HIF-2α, VHL, cullin2, NEDD8, PHD1, PHD2, PHD3, Siah1/2, cleaved caspase (e.g. caspase-3), syncytin, Fas, VEGF, FIH, and/or p53, and/or polynucleotides encoding same. The control can comprise markers derived from a pool of samples from individual patients with no disease, or individuals with a known condition.

The methods described herein may be performed by utilizing pre-packaged diagnostic kits comprising at least one specific Mtd-P Related Polynucleotide or binding agent (e.g. antibody) described herein, and optionally other markers, which may be conveniently used, e.g., in clinical settings, to screen and diagnose patients and to screen and identify those individuals exhibiting a predisposition to developing a disorder.

Nucleic acid-based detection techniques are described, below, in Section 4.1.1. Protein detection techniques are described, below, in Section 4.1.2.

The samples that may be analyzed using the methods of the invention include those which are known or suspected to express Mtd-P Related Polynucleotides or contain Mtd-P Related Polypeptides, and optionally other markers associated with the conditions disclosed herein. The samples may be derived from a patient or a cell culture, and include but are not limited to biological fluids, tissue extracts, freshly harvested cells, and lysates of cells which have been incubated in cell cultures. Examples of samples include tissues (e.g. placenta), extracts, or cell cultures, including cells, cell lysates, conditioned medium from maternal cells, and physiological fluids, such as, for example, whole blood, plasma, serum, saliva, ocular lens fluid, cerebral spinal fluid, sweat, urine, milk, ascites fluid, amniotic fluid, vaginal fluid, synovial fluid, peritoneal fluid and the like.

A sample can be used directly as obtained from the source or following a pretreatment to modify the character of the sample. Therefore, a sample can be treated prior to use, such as preparing plasma from blood, diluting viscous fluids, and the like. Methods of treatment can involve filtration, distillation, extraction, concentration, inactivation of interfering components, the addition of reagents, and the like. Polypeptides and polynucleotides may be isolated from the samples and utilized in the methods of the invention.

In embodiments of the invention the sample is a mammalian sample, preferably human sample. In another embodiment the sample is a physiological fluid such as serum or tissue (e.g. placenta).

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA; see, for example, Linsley & Schelter, U.S. patent application Ser. No. 09/411,074, or U.S. Pat. Nos. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Molecular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+ mRNA is eluted from the affinity column using 2 mM EDTA/0.1% SDS.

A sample of RNA can comprise a plurality of different mRNA molecules each with a different nucleotide sequence. In an aspect of the invention, the mRNA molecules in the RNA sample comprise at least 100 different nucleotide sequences.

### 4.1.1 Polynucleotide Methods

A condition mediated by a Mtd-P Related Polypeptide and/or Mtd-P Related Polynucleotide, a condition requiring regulation of trophoblast cell death, differentiation, invasion, and/or cell fusion and turnover (e.g., preeclampsia), or stage or type of same, may be detected based on the level of Mtd-P Related Polynucleotides in a sample. Techniques for detecting polynucleotides such as polymerase chain reaction (PCR) and hybridization assays are well known in the art.

Probes may be used in hybridization techniques to detect genes that encode Mtd-P Related Polypeptides. The technique generally involves contacting and incubating polynucleotides (e.g. recombinant DNA molecules, cloned genes) obtained from a sample from a patient or other cellular source with a probe under conditions favourable for the specific annealing of the probes to complementary sequences in the polynucleotides. After incubation, the non-annealed nucleic acids are removed, and the presence of polynucleotides that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a Mtd-P Related Polynucleotide, preferably they comprise 10-30, 10-40, 15-40, 20-50, 40-80, 50-150, or 80-120 nucleotides.

A nucleotide probe may be labelled with a detectable substance such as a radioactive label which provides for an adequate signal and has sufficient half-life such as ³²P, ³H, ¹⁴C or the like. Other detectable substances which may be used include antigens that are recognized by a specific labelled antibody, fluorescent compounds, enzymes, antibodies specific for a labelled antigen, and luminescent compounds. An appropriate label may be selected having regard to the rate of hybridization and binding of the probe to the nucleotide to be detected and the amount of nucleotide available for hybridization. Labelled probes may be hybridized to nucleic acids on solid supports such as nitrocellulose filters or nylon membranes as generally described in Sambrook et al, 1989, Molecular Cloning, A Laboratory Manual (2nd ed.).

The nucleic acid probes may be used to detect genes, preferably in human cells, that encode Mtd-P Related Polypeptides. The nucleotide probes may also be useful in the diagnosis of disorders involving a Mtd-P Related Polypeptide or conditions requiring regulation of trophoblast cell death, differentiation, invasion, and/or cell fusion and turnover (e.g., preeclampsia); in monitoring the progression of such disorders or conditions; or monitoring a therapeutic treatment. In aspects of the invention the nucleotide probes are useful in the diagnosis, prediction, management and control of preeclampsia involving one or more Mtd-P Related Polynucleotides, in monitoring the progression of preeclampsia; or monitoring a therapeutic treatment.

The levels of mRNA or polynucleotides derived therefrom can be determined using hybridization methods known in the art. For example, RNA can be isolated from a sample and separated on a gel. The separated RNA can then be transferred to a solid support and nucleic acid probes representing one or more markers can be hybridized to the solid support and the amount of marker-derived RNA can be determined. Such determination can be visual, or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization, or the lack thereof, of the labeled RNA to the solid support oligonucleotides is determined visually or by densitometer.

The detection of Mtd-P Related Polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art. By way of example, at least two oligonucleotide primers may be employed in a PCR based assay to amplify a portion of a Mtd-P Related Polynucleotide(s) derived from a sample, wherein at least one of the oligonucleotide primers is specific for (i.e. hybridizes to) a Mtd-P Related Polynucleotide. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a Mtd-P Related Polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of Mtd-P Related Polynucleotide expression. For example, RNA may be isolated from a cell type or tissue known to express a Mtd-P Related Polynucleotide and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a disorder involving a Mtd-P Related Polypeptide or a condition requiring regulation of trophoblast cell death, differentiation, invasion, and/or cell fusion and turnover (e.g., preeclampsia).

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynsli and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a Mtd-P Related Polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

In an embodiment, the invention provides a method wherein Mtd-P Related Polynucleotides that are mRNA are detected by (a) isolating mRNA from a sample and combining the mRNA with reagents to convert it to cDNA; (b) treating the converted cDNA with amplification reaction reagents and nucleic acid primers that hybridize to a Mtd-P Related Polynucleotide, to produce amplification products; (d) analyzing the amplification products to detect an amount of Mtd-P Related Polynucleotide mRNA; and (e) comparing the amount of mRNA to an amount detected against a panel of expected values for normal subjects derived using similar nucleic acid primers.

Mtd-P Related Polynucleotide-positive samples or alternatively higher levels in patients compared to a control (e.g. normal sample) may be indicative of a condition, (e.g., preeclampsia), and/or that the patient is not responsive to or tolerant of a therapy. Alternatively, negative samples or lower levels compared to a control (e.g. normal samples or negative samples) may also be indicative of a condition, and/or that a patient is not responsive to or tolerant of a therapy.

Amplification may be performed on samples obtained from a subject with a suspected condition described herein (e.g. suspected preeclampsia) and an individual who is not predisposed to such condition. The reaction may be performed on several dilutions of cDNA spanning at least two orders of magnitude. A significant difference in expression in several dilutions of the subject sample as compared to the same dilutions of the normal sample may be considered positive for the presence of the condition (e.g. preeclampsia).

Genotyping techniques known to one skilled in the art can be used to type polymorphisms that are in close proximity to the mutations in a gene encoding a Mtd-P Related Polypeptide. The polymorphisms may be used to identify individuals in families that are likely to carry mutations. If a polymorphism exhibits linkage disequilibrium with mutations in a Mtd-P gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

The primers and probes may be used in the above-described methods in situ i.e. directly on tissue sections (fixed and/or frozen) of patient tissue obtained from biopsies or resections.

Oligonucleotides or longer fragments derived from Mtd-P Related Polynucleotides may be used as targets in a micro-array. The micro-array can be used to simultaneously monitor the expression levels of Mtd-P Related Polynucleotides. The micro-array can also be used to identify genetic variants, mutations, and polymorphisms. The information from the micro-array may be used to determine gene function, to understand the genetic basis of a condition (e.g. preeclampsia), to diagnose a condition (e.g. preclampsia), and to develop and monitor the activities of therapeutic agents. Thus, the invention also includes an array comprising one or more Mtd-P Related Polynucleotides, and optionally other markers. The array can be used to assay expression of Mtd-P Related Polynucleotides in the array. The invention allows the quantitation of expression of one or more Mtd-P Related Polynucleotides. Arrays are also useful for ascertaining differential expression patterns of Mtd-P Related Polynucleotides as described herein, and optionally other markers, in normal and abnormal samples. This may provide a battery of nucleic acids that could serve as molecular targets for diagnosis or therapeutic intervention.

The preparation, use, and analysis of micro-arrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (1995) PCT application WO95/35505; Heller, R A. et al. (1997) Proc. Natl. Acad. Sci. 94:2150-2155; and Heller, M. J. et al. (1997) U.S. Pat. No. 5,605,662.). A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293:2101 (2001)].

Thus, the invention also includes an array comprising one or more markers associated with trophoblast cell death, differentiation, invasion, and/or cell fusion and turnover including without limitation Mtd-P, Mtd-L, Mtd-S, Mcl-1 isoforms (in particular Mcl-1S or Mcl-1L, or caspase cleaved McI-1S or Mcl-1L, in particular caspase cleaved Mcl-1L), TGFβ3, HIF-1α, HIF-2α, HIF-1β, VHL, PHD1, PHD2, PHD3, Siah1/2, VEGF, FIH, syncytin, cleaved caspase (e.g. caspase-3), cullin 2, NEDD8, Fas, and/or p53. The array can be used to assay expression of the markers in the array. The invention allows the quantitation of expression of one or more markers.

The invention provides microarrays comprising a disclosed marker set. In one embodiment, the invention provides a microarray for distinguishing conditions requiring modulation of trophoblast cell death, differentiation, invasion, and/or cell fusion and turnover comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality comprising or consisting essentially of at least 2, 3, 4, 5, 6, 7, 8, 9, 10, 11, 12, 13, or 14 of the genes corresponding to the markers Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF-1α, HJF-1β, HIF-2α, VHL, cleaved caspase (e.g. caspase-3), PHD1, PHD2, PHD3, Siah1/2, syncytin, VEGF, FIH, cullin 2, NEDD8, Fas, and/or p53. An aspect of the invention provides microarrays comprising at least 5, 10, or 14 of the polynucleotides encoding the markers.

The invention provides gene marker sets that distinguish conditions disclosed herein and uses therefor. In an aspect, the invention provides a method for classifying a condition disclosed herein comprising detecting a difference in the expression of a plurality of genes relative to a control, the plurality of genes consisting of at least 5 of the genes encoding the markers Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF-1α, HIF-2α, HIF-1β, VHL, cleaved caspase (e.g. caspase-3), PHD1, PHD2, PHD3, Siah1/2, syncytin, VEGF, FIH, cullin 2, NEDD8, Fas, and/or p53. In specific aspects, the plurality of genes consists of at least 10 or 15 of the genes encoding the markers Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF-1α, HIF-2α, HIF-1β, VHL, cleaved caspase (e.g. caspase-3), PHD1, PHD2, PHD3, Siah1/2, syncytin, VEGF, FIH, cullin 2, NEDD8, Fas, and/or p53. In another specific aspect, the control comprises nucleic acids derived from a pool of samples from individual control patients.

The invention provides a method for classifying condition disclosed herein by calculating the similarity between the expression of at least 5 polynucleotides encoding markers comprising or selected from the group consisting of Mtd-P, Mtd-L, Mtd-S, Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF-1α, HIF-2α, HIF-1β, VHL, cleaved caspase (e.g. caspase-3), PHD1, PHD2, PHD3, Siah1/2, syncytin, VEGF, FIH, cullin 2, NEDD8, Fas, and/or p53 in a sample to the expression of the same markers in a control pool, comprising the steps of:
(a) labeling nucleic acids derived from a sample, with a fluorophore to obtain a first pool of fluorophore-labeled nucleic acids;
(b) labeling with a second fluorophore a first pool of nucleic acids derived from two or more disease samples, and a second pool of nucleic acids derived from two or more control samples;
(c) contacting the first fluorophore-labeled nucleic acid and the first pool of second fluorophore-labeled nucleic acid with a first microarray under conditions such that hybridization can occur, and contacting the first fluorophore-labeled nucleic acid and the second pool of second fluorophore-labeled nucleic acid with a second microarray under conditions such that hybridization can occur, detecting at each of a plurality of discrete loci on the first microarray a first fluorescent emission signal from the first fluorophore-labeled nucleic acid and a second fluorescent emission signal from the first pool of second fluorophore-labeled genetic matter that is bound to the first microarray and detecting at each of the marker loci on the second microarray the first fluorescent emission signal from the first fluorophore-labeled nucleic acid and a third fluorescent emission signal from the second pool of second fluorophore-labeled nucleic acid;
(d) determining the similarity of the sample to patient and control pools by comparing the first fluorescence emission signals and the second fluorescence emission signals, and the first emission signals and the third fluorescence emission signals; and
(e) classifying the sample as from a individual with a condition where the first fluorescence emission signals are more similar to the second fluorescence emission signals than to the third fluorescent emission signals, and classifying the sample as from an individual without the condition where the first fluorescence emission signals are more similar to the third fluorescence emission signals than to the second fluorescent emission signals, wherein the fast microarray and the second microarray are similar to each other, exact replicas of each other, or are identical, and wherein the similarity is defined by a statistical method such that the cell sample and control are similar where the p value of the similarity is less than 0.01.

In an embodiment, the array can be used to monitor the time course of expression of one or more markers in the array. This can occur in various biological contexts such as disease progression. The array is also useful for ascertaining differential expression patterns of markers, and optionally other markers, in normal and abnormal cells. This may provide a battery of nucleic acids that could serve as molecular targets for diagnosis or therapeutic intervention.

Microarrays typically contain at separate sites nanomolar quantities of individual genes, cDNAs, or ESTs on a substrate (e.g. nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays can be prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect of the invention, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

In accordance with embodiments of the invention, a microarray is provided comprising a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In particular embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the markers is present on the array. In a preferred embodiment, the array comprises at least 5 of the markers disclosed herein.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridizae to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are produced by synthesizing polynucleotide probes on a support wherein the nucleotide probes are attached to the support covalently at either the 3′ or the 5′ end of the polynucleotide.

### 4.1.2 Polypeptide Methods

A Mtd-P Related Polypeptide may be detected using a binding agent “Binding agent” refers to a substance such as a polypeptide or antibody that specifically binds to one or more polypeptide disclosed herein (e.g. Mtd-P Related Polypeptide). A substance “specifically binds” to one or more polypeptide (e.g. Mtd-P Related Polypeptide) if is reacts at a detectable level with one or more polypeptide, and does not react detectably with peptides containing an unrelated or different sequence. Binding properties may be assessed using an ELISA, which may be readily performed by those skilled in the art (see for example, Newton et al, Develop. Dynamics 197: 1-13, 1993). A binding agent may be a ribosome, with or without a peptide component, an aptamer, an RNA molecule, or a polypeptide. A binding agent may be a polypeptide that comprises one or more polypeptide sequence (e.g. Mtd-P Related Polypeptide sequence), a peptide variant thereof, or a non-peptide mimetic of such a sequence. An aptamer includes a DNA or RNA molecule that binds to nucleic acids and proteins. An aptamer that binds to a protein (or binding domain) or a polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a Mtd-P Polypeptide (and optionally other markers) in a subject may be determined by (a) contacting a sample from the subject with a binding agent that interacts with a Mtd-P Related Polypeptide (and optionally binding agents that interact with other markers); (b) detecting in the sample a level of polypeptide or complex that binds to the binding agent(s); and (c) comparing the level(s) of polypeptide(s) with a predetermined standard or cut-off value.

In the context of certain methods of the invention, a sample, binding agents (e.g. antibodies specific for one or more Mtd-P Related Polypeptide), may be immobilized on a carrier or support. For example, an antibody or sample may be immobilized on a carrier or solid support which is capable of immobilizing cells, antibodies etc. Suitable carriers or supports may comprise nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Immobilization typically entails separating the binding agent from any free analytes (e.g. free Mtd-P Related Polypeptide or free PSF Complex) in the reaction mixture.

Binding agents may be labeled using conventional methods with a detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy. Where a radioactive label is used as a detectable substance, a Mtd-P Related Polypeptide may be localized by radioautography. The results of radioautography may be quantitated by determining the density of particles in the radioautographs by various optical methods, or by counting the grains.

Binding agents, including antibodies to a Mtd-P Related Polypeptide or protein complex comprising a Mtd-P Related Polypeptide, or peptides that interact with a Mtd-P Related Polypeptide or complex thereof, may also be indirectly labeled with a ligand binding partner. For example, the antibodies, or peptides may be conjugated to one partner of a ligand binding pair, and the polypeptide may be coupled to the other partner of the ligand binding pair. Representative examples include avidin-biotin, and riboflavin-riboflavin binding protein. In an embodiment the binding agent (e.g. antibodies) are biotinylated. Methods for conjugating binding agents such as antibodies with a ligand binding partner may be readily accomplished by one of ordinary skill in the art (see Wilchek and Bayer, “The Avidin-Biotin Complex in Bioanalytical Applications,” Anal. Biochem. 171:1-32, 1988).

A binding agent can directly or indirectly interact with a Mtd-P Related Polypeptide. Indirect methods may be employed in which a primary binding agent-binding partner interaction is amplified by introducing a second agent. In particular, a primary Mtd-P Related Polypeptide-antibody reaction may be amplified by the introduction of a second antibody, having specificity for the antibody reactive against Mtd-P Related Polypeptides. By way of example, if the antibody having specificity against a Mtd-P Related Polypeptide is a rabbit IgG antibody, the second antibody may be goat anti-rabbit gamma-globulin labeled with a detectable substance as described herein.

The presence of a Mtd-P Polypeptide may be determined by measuring the binding of the Mtd-P Related Polypeptide to molecules (or parts thereof) which are known to interact with a Mtd-P Related Polypeptide. In aspects of the invention, peptides derived from sites on a polypeptide which binds to a Mtd-P Related Polypeptide may be used. A peptide derived from a specific site on a binding polypeptide may encompass the amino acid sequence of a naturally occurring binding site, any portion of that binding site, or other molecular entity that functions to bind an associated molecule. A peptide derived from such a site will interact directly or indirectly with an associated molecule in such a way as to mimic the native binding site. Such peptides may include competitive inhibitors, enhancers, peptide mimetics, and the like as discussed herein.

In other aspects of the invention, the binding agent is an antibody. Antibodies specifically reactive with a Mtd-P Related Polypeptide, or derivatives, such as enzyme conjugates or labelled derivatives, may be used to detect Mtd-P Related Polypeptides in various samples (e.g. biological materials). They may be used as diagnostic or prognostic reagents and they may be used to detect abnormalities in the level of Mtd-P Related Polypeptide expression, or abnormalities in the structure, and/or temporal, tissue, cellular, or subcellular location of a Mtd-P Related Polypeptide. Antibodies may also be used to screen potentially therapeutic compounds in vitro to determine their effects on disorders involving a Mtd-P Related Polypeptide, and other conditions. In vitro immunoassays may also be used to assess or monitor the efficacy of particular therapies. The antibodies of the invention may also be used in vitro to determine the level of Mtd-P Related Polynucleotides expression in cells genetically engineered to produce a Mtd-P Related Polypeptide.

In particular the invention provides a diagnostic method for monitoring or diagnosing a condition involving or mediated by a Mtd-P Related Polypeptide in a subject by quantitating Mtd-P Related Polypeptides or complexes thereof in a biological sample from the subject comprising reacting the sample with antibodies specific for Mtd-P Related Polypeptides or complexes thereof, which are directly or indirectly labeled with detectable substances and detecting the detectable substances. In a particular embodiment of the invention, Mtd-P Related Polypeptides are quantitated or measured.

In an aspect of the invention, a method for detecting a condition mediated by a Mtd-P Related Polypeptide is provided comprising:
(a) obtaining a sample suspected of containing Mtd-P Related Polypeptides or complexes thereof;
(b) contacting the sample with antibodies that specifically bind to the Mtd-P Related Polypeptides or complexes thereof under conditions effective to bind the antibodies and form complexes;
(c) measuring the amount of Mtd-P Related Polypeptides or complexes thereof present in the sample by quantitating the amount of the antibody-Mtd-P Related Polypeptides or antibody-Mtd-P complexes; and
(d) comparing the amount of Mtd-P Related Polypeptides or complexes thereof present in the samples with the amount of Mtd-P Related Polypeptides or complexes thereof in a control, wherein a change or significant difference in the amount of Mtd-P Related Polypeptides or complexes thereof in the sample compared with the amount in the control is indicative of the condition.

The amount of antibody complexes may also be compared to a value representative of the amount of antibody complexes from an individual not at risk of, or afflicted with, a condition or having a condition at different stages. A significant difference in antibody complex formation may be indicative of an advanced condition, or an unfavourable prognosis.

In embodiments of the methods of the invention, Mtd-P Related Polypeptides or complexes thereof are detected in samples and higher levels, in particular significantly higher levels compared to a control (e.g. normal) is indicative of a condition (e.g. preeclampsia).

In an embodiment, the invention contemplates a method for monitoring the progression of a condition mediated by a Mtd-P Related Polypeptide in an individual, comprising:
(a) contacting antibodies which bind to Mtd-P Related Polypeptides or complexes thereof with a sample from the individual so as to form complexes comprising the antibodies and Mtd-P Related Polypeptides or complexes thereof in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of a condition, condition stage, and/or progression of the condition in the individual.

In methods of the invention the step of contacting a sample with a binding agent (e.g. antibodies) may be accomplished by any suitable technique so that detection can occur. In particular, antibodies may be used in any known immunoassays that rely on the binding interaction between antigenic determinants of one or more Mtd-P Related Polypeptides or complexes thereof and the antibodies. Immunoassay procedures for in vitro detection of antigens in fluid samples are well known in the art, as well as widely established and used in the commercial diagnostic industry. [See for example, Paterson et al., Int. J. Can. 37:659 (1986) and Burchell et al., Int. J. Can. 34:763 (1984) for a general description of immunoassay procedures]. Qualitative and/or quantitative determinations of Mtd-P Related Polypeptides or complexes thereof in a sample may be accomplished by competitive or non-competitive immunoassay procedures in either a direct or indirect format. Detection of Mtd-P Related Polypeptides or complexes thereof using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

Thus, the present invention provides means for determining one or more Mtd-P Related Polypeptides in a sample by measuring one or more Mtd-P Related Polypeptides by immunoassay. According to an embodiment of the invention, an immunoassay for detecting Mtd-P Related Polypeptides in a biological sample comprises contacting antibodies that specifically bind to Mtd-P Related Polypeptides or complexes thereof in the sample under conditions that allow the formation of first complexes comprising antibodies and Mtd-P Related Polypeptides or complexes and determining the presence or amount of the first complexes as a measure of the amount of Mtd-P Related Polypeptides or complexes contained in the sample.

It will be evident to a skilled artisan that a variety of immunoassay methods can be used to measure one or more Mtd-P Related Polypeptides. In general, an immunoassay method may be competitive or noncompetitive.

In an aspect of the invention a competitive method is provided employing an immobilized or immobilizable antibody to a Mtd-P Related Polypeptide and a labeled form of a Mtd-P Related Polypeptide. Sample Mtd-P Related Polypeptides and labeled Mtd-P Related Polypeptides compete for binding to antibodies to Mtd-P Related Polypeptides. After separation of the resulting labeled Mtd-P Related Polypeptides that have become bound to antibodies (bound fraction) from that which has remained unbound (unbound fraction), the amount of the label in either bound or unbound fraction is measured and may be correlated with the amount of Mtd-P Related Polypeptides in the test sample in any conventional manner, e.g., by comparison to a standard curve.

In another aspect, a non-competitive method is used for the determination of Mtd-P Related Polypeptides, with the most common method being the “sandwich” method. In this assay, two antibodies to Mtd-P Related Polypeptides are employed. One of the antibodies to Mtd-P Related Polypeptides is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises Mtd-P Related Polypeptides bound by (“sandwiched” between) the capture and detection antibodies. In an embodiment, the label may be measured without separating the capture antibodies and liquid test mixture.

The above-described immunoassay methods and formats are intended to be exemplary and are not limiting. Other methods now or hereafter developed for the determination of a Mtd-P Related Polypeptide or complexes thereof are included within the scope hereof.

Binding agents (e.g. antibodies) may be used to detect and quantify one or more Mtd-P Related Polypeptides or complexes in a sample in order to diagnose and treat pathological states. In particular, antibodies may be used in immunohistochemical analyses, for example, at the cellular and sub-subcellular level, to detect one or more Mtd-P Related Polypeptides, to localize them to particular cells and tissues and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a condition described herein is contacted with antibodies, preferably monoclonal antibodies recognizing Mtd-P Related Polypeptides. The site at which the antibodies are bound is determined by selective staining of the sample by standard immunohistochemical procedures. The tissue sample may be normal tissue or abnormal/disease tissue.

Antibodies specific for one or more Mtd-P Related Polypeptides or complexes may be labelled with a detectable substance as described herein and localised in tissues and cells based upon the presence of the detectable substance. Cytochemical techniques known in the art for localizing antigens using light and electron microscopy may be used to detect Mtd-P Related Polypeptides or complexes thereof

### 4.1.3 Diagnostic Methods for Conditions Involving Trophoblast Invasion

The invention in particular contemplates diagnostic methods for conditions associated with trophoblast cell death, differentiation, invasion, and/or cell fusion and turnover.

In an aspect, the invention provides methods for determining the presence or absence of a condition associated with trophoblast cell death, differentiation, invasion, and/or cell fusion and turnover, in a subject comprising (a) contacting a sample obtained from the subject with oligonucleotides that hybridize to Mtd polynucleotides, in particular Mtd-P Related Polynucleotides, and (b) detecting in the sample levels of polynucleotides that hybridize to the Mtd-P polynucleotides relative to a predetermined cut-off value or standard, and therefrom determining the presence or absence of the condition in the subject. In a particular aspect, the Mtd polynucleotides are polynucleotides encoding Mtd-P, Mtd-L, and Mtd-S, more particularly Mtd-P and Mtd-L, most particularly Mtd-P of SEQ ID NO. 1 and Mtd-L of SEQ ID NO. 3, preferably the polynucleotides of SEQ ID NO. 2 and 4. In an embodiment of the diagnostic method of the invention, a method is provided for diagnosing increased risk of preeclampsia in a subject comprising detecting Mtd-P Related Polynucleotides, in particular a Mtd-P polynucleotide of SEQ ID NO. 2 in a sample from the subject.

The present invention also provides a method for diagnosing in a subject a condition associated with trophoblast cell death, differentiation, invasion, and/or cell fusion and turnover and/or requiring regulation of trophoblast invasion, comprising detecting one or more Mtd polypeptide in a sample from the subject. In an aspect, the Mtd polypeptide is Mtd-L, Mtd-S and/or a Mtd-P Related Polypeptide, in particular a Mtd-P of SEQ ID NO. 1 and/or Mtd-L of SEQ ID No. 3. In an embodiment of the diagnostic method of the invention, a method is provided for diagnosing increased risk of preeclampsia in a subject comprising detecting Mtd-P Related Polypeptides, in particular Mtd-P of SEQ ID NO. I in a sample from the subject.

The diagnostic methods of the invention may optionally or alternatively detect other markers associated with trophoblast cell death, differentiation, invasion, and/or cell fusion and turnover or markers involved in modulating hypoxia. In aspects of the invention the other markers include without limitation Mcl-1 isoforms (in particular Mcl-1S or Mcl-1L, or caspase cleaved Mcl-1S or Mcl-1L, in particular caspase cleaved Mcl-1L), TGFβ3, TGFβ1, HIF1α, HIF1β, HIF2α, PHD1, PHD2, PHD3, VHL, Siah1/2, syncytin, cullin 2, cleaved caspase (e.g. caspase-3), VEGF, FIH, NEDD8, Fas, and/or p53, or polynucleotides encoding same. Thus, a diagnostic method of the invention may detect multiple markers using methods similar to those described herein for Mtd-P Related Polypeptides and Mtd-P Related Polynucleotides. In an aspect the markers can be contained on a nucleic acid or protein micro-array.

The diagnostic methods disclosed herein can be used to determine the presence or absence of preeclampsia or to determine the likelihood of occurrence of preeclampsia in a subject, or to distinguish subpathologies, including early onset preeclampsia from late onset preclampsia, or intrauterine growth restriction (IUGR).

In an aspect, the invention contemplates a method for determining the likelihood of occurrence of preeclampsia in a pregnant mammal comprising detecting a Mtd-P Related Polypeptide and/or Mtd-P Related Polynucleotide in a sample from the subject.

In an embodiment of the invention, a method is provided for diagnosing increased risk of preeclampsia in a subject comprising detecting a Mtd polypeptide, in particular Mtd-P of SEQ ID NO. 1 in a sample, and in particular using antibodies specific for a Mtd Polypeptide, in particular Mtd-P of SEQ ID NO. 1. Levels of a Mtd Polypeptide, in particular Mtd-P of SEQ ID NO. 1 may be measured during the first trimester of pregnancy (approximately 1 to 15 weeks, 1 to 14 weeks, 1 to 12 weeks, 5 to 12 weeks, 5 to 8 weeks, 9 to 12 weeks, or 10 to 15 weeks). At least two measurements may be taken during this period, and subsequently including a measurement at about 12 to 16 or 14 to 16 weeks. If the levels are significantly different (e.g., increased) as compared to levels typical for women who do not suffer from preeclampsia, the patient is diagnosed as having an increased risk of suffering preeclampsia. Levels significantly different from (e.g. above) those typical for women who do not suffer from preeclampsia may be suspect and further monitoring and measurement of a Mtd polypeptide, in particular Mtd-P of SEQ ID NO. 1 may be appropriate. The information from the diagnostic method may be used to identify subjects who may benefit from a course of treatment, such as treatment via administration of inhibitors of a Mtd Polypeptide, in particular Mtd-P of SEQ ID NO. 1 as discussed herein.

In a method for diagnosing or identifying early severe onset preeclampsia, higher levels of the markers, in particular significantly higher levels of one or more Mtd polynucleotides, more particularly polynucleotides encoding Mtd-L and Mtd-P, most particularly polynucleotides encoding Mtd-L of SEQ ID NO. 3 and Mtd-P of SEQ ID NO. 1, in patients compared to a control (e.g. normal) are indicative of early severe onset preeclampsia, or the likelihood of occurrence of early severe onset preeclampsia. Thus, the invention relates to a method for diagnosing early onset preeclampsia in a subject comprising detecting Mtd-P Related Polynucleotides, in particular a Mtd-P Polynucleotide of SEQ ID NO. 2, and/or a polynucleotide encoding Mtd-L, in particular a Mtd-L polynucleotide of SEQ ID NO. 4, in a sample from the subject. The method may optionally comprise detecting polynucleotides encoding Mcl-1 isoforms (in particular Mdl-1S or Mcl-1L, or caspase cleaved Mcl-1S or Mcl-1L, in particular caspase cleaved Mcl-1L, for example SEQ ID NOs. 7, 8, and 10), TGFβ3, TGFβ1, HIF1α, HIF1β, HIF2α, PHD1, PHD2, PHD3, VHL, Siah1/2, syncytin, cullin 2, cleaved caspase (e.g. caspase-3), VEGF, FIH, NEDD8, Fas, and/or p53. The diagnostic methods can comprise diagnosing early onset preeclampsia using a panel of markers comprising or selected from the group consisting of a Mtd polynucleotide (in particular Mtd-P and Mtd-L), and polynucleotides encoding Mcl-1 isoforms (in particular Mcl-1S or Mcl-1L, or caspase cleaved Mcl-1S or Mcl-1L, in particular caspase cleaved Mcl-1L, for example SEQ ID NOs. 7, 8, and 10), TGFβ3, TGFβ1, HIF1α, HIF1β, HIF2α, PHD1, PHD2, PHD3, VHL, Siah1/2, syncytin, cullin 2, cleaved caspase (e.g. caspase-3), VEGF, FIH, NEDD8, Fas, and/or p53.

In another method for diagnosing or identifying early severe onset preeclampsia, higher levels of the markers, in particular significantly higher levels of one or more Mtd polypeptides, more particularly Mtd-L and Mtd-P, most particularly Mtd-L of SEQ ID NO. 3 and Mtd-P of SEQ ID NO. 1, in patients compared to a control (e.g. normal) are indicative of early severe onset preeclampsia, or the likelihood of occurrence of early severe onset preeclampsia. Thus, the invention relates to a method for diagnosing early onset preeclampsia in a subject comprising detecting Mtd-P Related Polypeptides, in particular Mtd-P of SEQ ID NO. 1, and/or Mtd-L, in particular Mtd-L of SEQ ID NO. 3 in a sample from the subject. The method may optionally comprise detecting Mcl-1 isoforms (in particular Mcl-1S or Mcl-1L, or caspase cleaved Mcl-1S or Mcl-1L, in particular caspase cleaved Mcl-1L, for example SEQ ID NOs. 5, 6 and 9), TGFβ3, TGFβ1, HIF1α, HIF1β, HIF2α, PHD1, PHD2, PHD3, VHL, syncytin, cullin 2, NEDD8, FIH, VEGF, Siah1, Siah2, Fas, cleaved caspase (e.g. caspase-3), and/or p53. The diagnostic methods can comprise diagnosing early onset preeclampsia using a panel of markers comprising or selected from the group consisting of a Mtd polypeptide (in particular Mtd-P and Mtd-L), and Mcl-1 isoforms (in particular Mcl-1S or Mcl-1L, or caspase cleaved Mcl-1S or Mcl-1L, in particular caspase cleaved Mcl-1L, for example SEQ ID NOs. 5, 6, and 9), TGFβ3, TGFα1, HIF1α, HIF1β, HIF2α, PHD 1, PHD2, PHD3, VHL, syncytin, cullin 2, NEDD8, FIH, VEGF, Siah1, Siah2, Fas, cleaved caspase (e.g. caspase-3), and/or p53. In an aspect of a diagnostic method for preeclampsia, one or more of the levels of Mtd-P, Mtd-L, Mcl-1S and Mcl-1L and truncations thereof, TGFβ3, NEDD8, and cullin 2 are increased, and one or more of the levels of PHD1, PHD2, VHL, Siah1, and Siah2 are decreased compared to a control.

The invention provides a method for diagnosing early onset preeclampsia comprising comparing levels of at least two, three, four, five, six, seven, eight, nine or ten of Mtd-P, Mtd-L, Mcl-1S, Mcl-1L, Mcl-1L truncation, TGFβ3, HIF1α, PHD1, PHD2, NEDD8, cullin 2, cleaved caspase (e.g. caspase-3), Siah1/2, and VHL, or polynucleotides encoding same in a sample from a subject to the corresponding levels in a control. In a particular embodiment, the invention provides a method for diagnosing early onset preeclampsia comprising comparing levels of at least two, three, four, five, six, seven, eight, nine, or ten of Mtd-P, Mtd-L, Mcl-1S, Mcl-1L, Mcl-1L cleaved by caspase, TGFβ3, HIF1α, PHD1, PHD2, NEDD8, cullin 2, cleaved caspase (e.g. caspase-3), Siah1/2, and VHL, or polynucleotides encoding same in a sample taken from a subject in the first trimester of pregnancy, in particular before 14, 12, 10, 8, or 5 weeks, to the corresponding levels in a control. The control may be a pre-term or normotensive age-matched control or a sample taken at different stage of pregnancy. In a particular embodiment, a significant increase in Mtd-P or Mtd-L or polynucleotides encoding same and optionally TGFβ3 and/or HIF1α or polynucleotides encoding same is indicative of early onset preeclampsia. In another particular embodiment, a significant increase in Mtd-P and/or Mtd-L or polynucleotides encoding same and optionally TGFβ3 and/or HIF1α or polynucleotides encoding same, and/or a significant decrease in PHD1, PHD2, Siah1/2, NEDD8, cullin 2, and/or VHL or polynucleotides encoding same, is indicative of early onset preeclampsia. In another particular embodiment, a significant increase in Mtd-P or Mtd-L or polynucleotides encoding same, and/or a significant decrease in PHD1, PHD2 and/or VHL or polynucleotides encoding same, is indicative of early onset preeclampsia. In a further particular embodiment, a 2 to 10 fold, 2 to 8 fold, 2 to 5 fold, 2 to 4 fold, or 3 to 3.5 fold increase in Mtd-P or polynucleotide encoding same, and/or a 2 to 10 fold, 2 to 8 fold, 2 to 5 fold, 2 to 4 fold, or 2 to 3.6 fold increase in Mtd-L or polynucleotide encoding same, compared to a control is indicative of early onset preeclampsia.

The invention also provides a method for diagnosing late onset preeclampsia comprising comparing levels of Mtd-L polypeptides or polynucleotides encoding same in a sample from a subject to the corresponding levels in a control. The sample is generally taken from a subject in the third trimester of pregnancy, in particular after week 20 or 25.

The invention also provides a method for diagnosing late onset preeclampsia with intrauterine growth restriction (IUGR) comprising comparing levels of Mtd-L polypeptides or polynucleotides encoding same, and optionally HIF1α, VHL, TGFβ3, PHD1, PHD2, PHD3, Mtd-P, and/or Siah1/2, or polynucleotides encoding same in a sample from a subject to the corresponding levels in a control. The sample is generally taken from a subject in the third trimester of pregnancy, in particular after about week 20 or 25. An increase in Mtd-L levels, or polynucleotides encoding same, and optionally no change in HIF1α, VHL, TGFβ3, Mtd-P, PHD1, PHD2, PHD3, and/or Siah1/2 or polynucleotides encoding same, can be indicative of late onset preeclampsia with IUGR.

The invention also contemplates a method for monitoring the progression of preeclampsia in an individual, comprising:
(a) contacting an amount of binding agent (e.g., an antibody) which binds to a Mtd polypeptide (e.g. Mtd-L and/or a Mtd-P Related Polypeptide), with a sample from the individual so as to form a binary complex comprising the binding agent and Mtd Polypeptide in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the progression of the preeclampsia in said individual.

The amount of complexes may also be compared to a value representative of the amount of the complex. In an embodiment an increase in complexes in (c) is indicative of preeclampsia.

The invention further provides a method for diagnosing severe IUGR, comprising comparing levels of PHD1, PHD2, and/or PHD3, and optionally Siah1, Siah2, VEGF, FIH, and/or HIF1α or polynucleotides encoding same, in a sample from a subject to the corresponding levels in a control. An increase, in particular a significant increase in levels of one or both of PHD1 and PHD3, and optionally PHD2, Siah1, Siah2, and FIH, or polynucleotides encoding same, and/or decreased levels of VEGF or a polynucleotide encoding same can be indicative of IUGR. In an aspect the samples are taken from a subject at or later than about 20, 25 or 30 weeks.

It will also be appreciated that the diagnostic methods disclosed herein may also be useful in the diagnosis or monitoring of choriocarcinoma or hydatiform mole which involves uncontrolled trophoblast invasion and may be associated with abnormally low levels of a Mtd polypeptide, in particular a Mtd-P Related Polypeptide, more particularly Mtd-P of SEQ ID NO. 1, or polynucleotides encoding same. Further the above methods may be used to diagnose or monitor other pregnancy complications including molar pregnancy, preterm labour, preterm birth, fetal anomalies, and placental abruption.

In an aspect, the invention provides a method for diagnosing molar pregnancies comprising comparing levels of Mtd polypeptides (e.g. Mtd-L, Mtd-S, and/or Mtd-P), or polynucleotides encoding same in a sample from a subject to the corresponding levels in a control. An increase in Mtd polypeptides in general may be indicative of a molar pregnancy.

Polypeptides and polynucleotides may be detected in patient samples using the methods disclosed herein. In particular, a polypeptide to be analyzed in a method of the invention may be detected using a binding agent or a substance which directly or indirectly interacts with the polypeptide. For example, antibodies specific for a Mtd polypeptide, in particular Mtd-P of SEQ ID NO. 1, may be used to diagnose and monitor a condition associated with abherant trophoblast cell death, differentiation, invasion, and/or cell fusion and turnover or requiring regulation of trophoblast invasion. A method of the invention using antibodies may utilize Countercurrent immuno-Electrophoresis (CIEP), Radioimmunoassays, Radioimmunoprecipitations, and Enzyme-Linked Immuno-Sorbent Assays (ELISA), Dot Blot assays, Inhibition or Competition assays and sandwich assays as described herein and known in the art.

The presence of a Mtd polypeptide, in particular Mtd-P of SEQ ID NO. 1, and other polypeptides disclosed herein, in a sample may also be determined by measuring the binding of the Mtd polypeptide, in particular Mtd-P of SEQ ID NO. 1, or other polypeptides with substances that are known to bind to same. A binding agent, in particular antibodies specific for a Mtd polypeptide, more particularly Mtd-P of SEQ ID NO. 1, or other polypeptides disclosed herein, may be labeled using conventional methods with various detectable substances such as enzymes, fluorescent materials, luminescent materials and radioactive materials which are described herein and known to a person skilled in the art. A binding agent (e.g. an antibody to a Mtd polypeptide, in particular Mtd-P of SEQ ID NO. 1) may also be indirectly labeled with a ligand binding partner. For example, the antibodies, or a binding agent may be conjugated to one partner of a ligand binding pair, and the polypeptide (e.g. Mtd polypeptide, in particular Mtd-P of SEQ ID NO. 1), may be coupled to the other partner of the ligand binding pair. Representative examples of binding partners are described herein.

The antibodies or binding agents used in the method of the invention may be insolubilized as described herein. Indirect methods may also be employed in which a primary antigen-antibody reaction is amplified by the introduction of a second antibody, having specificity for the antibody reactive against the cytokine.

A polypeptide to be detected in a diagnostic method of the invention, in particular Mtd-P of SEQ ID NO. 1 or a polynucleotide encoding same, can be assayed in a sample using nucleotide probes to detect polynucleotides encoding the polypeptide, (e.g. Mtd-P of SEQ ID NO. 2). Suitable probes include polynucleotides based on nucleic acid sequences encoding the polypeptide (e.g. a Mtd polypeptide, in particular Mtd-P of SEQ ID NO. 1). A nucleotide probe may be labeled with a detectable substance as described herein.

A polynucleotide (e.g. a polynucleotide encoding a Mtd polypeptide, in particular Mtd-P of SEQ ID NO. 1) can also be detected by selective amplification of the polynucleotide using polymerase chain reaction (PCR) methods. Synthetic oligonucleotide primers can be constructed from the sequences of a polynucleotide (e.g. Mtd polynucleotide, in particular Mtd-P of SEQ ID NO. 2) using conventional methods. A nucleic acid can be amplified in a sample using these oligonucleotide primers and standard PCR amplification techniques.

### 4.2 Kits

The invention also relates to kits for carrying out the methods of the invention. In an aspect, the invention provides a test kit for diagnosing a condition requiring regulation of trophoblast cell death, differentiation, invasion, and/or cell fusion and turnover, in particular preeclampsia, choriocarcinoma, hydatiform mole, or a molar pregnancy, which comprises a binding agent that interacts with a Mtd polypeptide, in particular a Mtd-P Related Polypeptide, and optionally one or more other polypeptide markers disclosed herein, or a polynucleotide that interacts with a Mtd polynucleotide, in particular a Mtd-P Polynucleotide, and optionally one or more other polynucleotide markers disclosed herein.

A kit can comprise instructions, negative and positive controls, and means for direct or indirect measurement of Mtd-P Related Polypeptides, or one or more other markers disclosed herein. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific polypeptide disclosed herein (e.g., Mtd polypeptide, in particular Mtd-P Related Polypeptide) or binding agent (e.g. antibody) described herein, which may be conveniently used, e.g., in clinical settings to screen and diagnose patients and to screen and identify those individuals exhibiting a predisposition to a condition disclosed herein, in particular preeclampsia.

In an embodiment, a container with a kit comprises one or more binding agent as described herein. By way of example, the kit may contain antibodies or antibody fragments which bind specifically to epitopes of Mtd polypeptides, in particular Mtd-P Related Polypeptides, and optionally other markers; antibodies against the antibodies labelled with an enzyme; and, a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more Mtd polypeptide (e.g. a Mtd-P Related Polypeptide comprising a sequence of SEQ ID NO. 1), and means for detecting binding of the antibodies to their epitope associated with a condition disclosed herein, either as concentrates (including lyophilized compositions), which may be further diluted prior to use or at the concentration of use, where the vials may include one or more dosages.

A kit may be designed to detect the level of polynucleotides encoding one or more Mtd polynucleotide, in particular Mtd-P Related Polynucleotides, and/or one or more other polynucleotide markers disclosed herein in a sample. In an embodiment, the polynucleotides encode one or more polynucleotides comprising a sequence of SEQ ID Nos. 2, 7 or 8. Such kits generally comprise at least one oligonucleotide probe or primer, as described herein, that hybridizes to a Mtd polynucleotide or other polynucleotide marker disclosed herein. Such an oligonucleotide may be used, for example, within a PCR or hybridization procedure.

The invention provides a kit containing a microarray described herein ready for hybridization to target Mtd Polynucleotides, in particular Mtd-P Related Polynucleotides, and optionally one or more other polynucleotide markers disclosed herein, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

In an aspect, the invention provides a kit comprising a reagent that detects a Mtd polypeptide, in particular a Mtd-P Related Polypeptide or a polynucleotide encoding a Mtd polypeptide, in particular a Mtd-P Related Polynucleotide, and instructions or package insert or label for assaying whether a pregnant mammal is at risk of early onset preeclampsia. The kit may further comprise a detection means and/or microtiter plates, a Mtd polypeptide or Mtd polynucleotide standard or tracer, which is typically labeled, and an immobilized reagent that detects Mtd polypeptide or Mtd polynucleotide, which is used to capture the Mtd polypeptide or Mtd polynucleotide.

The invention contemplates a kit for assessing the presence of cells and tissues associated with a condition disclosed herein, wherein the kit comprises antibodies specific for one or more Mtd polypeptide, in particular Mtd-P Related Polypeptide, or complexes thereof, or primers or probes for Mtd polynucleotides, in particular Mtd-P Related Polynucleotides, and optionally probes, primers or antibodies specific for other markers associated with the condition.

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting a condition disclosed herein. In an aspect, the kit comprises reagents for assessing one or more Mtd-P Related Polypeptides or Mtd-P Related Polynucleotides, and optionally a plurality of test agents or compounds.

Additionally the invention provides a kit for assessing the potential of a test compound to contribute to a condition disclosed herein. In an aspect, the kit comprises cells and tissues associated with the condition and reagents for assessing one or more Mtd-P Related Polypeptides or Mtd-P Related Polynucleotides, and optionally other markers associated with the condition.

### 4.3 Computer Systems

Analytic methods contemplated herein can be implemented by use of computer systems and methods described below and known in the art. Thus, the invention provides computer readable media comprising one or more markers including without limitation Mtd-P, Mtd-L, Mtd-S, Mcl-1 isoforms (in particular McI-1S or Mcl-1L, or caspase cleaved Mcl-1S or Mcl-1L, in particular caspase cleaved Mcl-1L), TGFβ3, TGFβ1, HIF-1α, HIF-2α, HIF-1β, VHL, cleaved caspase (e.g. caspase-3), PHD1, PHD2, PHD3, Siah1/2, VEGF, FIH, syncytin, cullin 2, NEDD8, Fas, and/or p53 and/or polynucleotides encoding one or more markers. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more markers disclosed herein.

A variety of data processor programs and formats can be used to store information on one or more markers, and/or polynucleotides encoding one or more markers. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has a condition disclosed herein or a pre-disposition to a condition disclosed herein, comprising determining the presence or absence of one or more markers disclosed herein, and/or polynucleotides encoding one or more markers, and based on the presence or absence of the markers, and/or polynucleotides, determining the condition or a pre-disposition to a condition, optionally recommending a procedure or treatment.

The invention also provides in an electronic system and/or in a network, a method for determining whether a subject has a condition disclosed herein, or a pre-disposition to a condition disclosed herein, comprising determining the presence or absence of one or more markers disclosed herein, and/or polynucleotides encoding one or more markers, and based on the presence or absence of the markers, and/or polynucleotides, determining whether the subject has the condition or a pre-disposition to the condition, and optionally recommending a procedure or treatment.

The invention further provides in a network, a method for determining whether a subject has a condition disclosed herein or a pre-disposition to a condition disclosed herein comprising: (a) receiving phenotypic information on the subject and information on one or more markers disclosed herein, and/or polynucleotides encoding one or more markers, associated with samples from the subject; (b) acquiring information from the network corresponding to the markers and/or polynucleotides; and (c) based on the phenotypic information and information on the markers and/or polynucleotides, determining whether the subject has the condition or a pre-disposition to the condition, and (d) optionally recommending a procedure or treatment.

The invention still further provides a system for identifying selected records that identify a diseased cell or tissue. A system of the invention generally comprises a digital computer; a database server coupled to the computer; a database coupled to the database server having data stored therein, the data comprising records of data comprising one or more markers disclosed herein, and/or polynucleotides encoding one or more markers, and a code mechanism for applying queries based upon a desired selection criteria to the data file in the database to produce reports of records which match the desired selection criteria.

The invention contemplates a business method for determining whether a subject has a condition disclosed herein or a pre-disposition to a condition disclosed herein comprising: (a) receiving phenotypic information on the subject and information on one or more markers disclosed herein, and/or polynucleotides encoding the markers, associated with samples from the subject; (b) acquiring information from a network corresponding to the markers and/or polynucleotides; and (c) based on the phenotypic information, information on the markers and/or polynucleotides, and acquired information, determining whether the subject has the condition or a pre-disposition to the condition, and optionally recommending a procedure or treatment.

In an aspect of the invention, the computer systems, components, and methods described herein are used to monitor a condition or determine the stage of a condition.

### 4.4 Methods for Identifying or Evaluating Substances/Compounds

The invention contemplates methods designed to identify substances that modulate the biological activity of a Mtd-P Related-Polypeptide, in particular Mtd-P of SEQ ID NO. 1 including substances that bind to a Mtd-P Related Polypeptide, in particular Mtd-P of SEQ ID NO. 1, or bind to other proteins that interact with a Mtd-P Related Polypeptide, to compounds that interfere with, or enhance the interaction of a Mtd-P Related Polypeptide, in particular Mtd-P of SEQ ID NO. 1, and substances that bind to a Mtd-P Related Polypeptide, in particular Mtd-P of SEQ D NO. 1, or other proteins that interact with a Mtd-P Related Polypeptide, in particular Mtd-P of SEQ ID NO. 1. Methods are also utilized that identify compounds that bind to regulatory sequences of a Mtd-P Related Polynucleotide.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound.

Substances identified using the methods of the invention may be isolated, cloned and sequenced using conventional techniques. A substance that associates with a Mtd-P Related Polypeptide of the invention may be an agonist or antagonist of the biological or immunological activity of the polypeptide.

The term “agonist”, refers to a molecule that increases the amount of, or prolongs the duration of, the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention.

Substances which modulate a Mtd-P Related Polypeptide can be identified based on their ability to bind to a Mtd-P Related Polypeptide. Therefore, the invention also provides methods for identifying substances which bind to a Mtd-P Related Polypeptide.

Substances which can bind with a Mtd-P Related Polypeptide may be identified by reacting a Mtd-P Related Polypeptide with a test substance which potentially binds to a Mtd-P Related Polypeptide, under conditions which permit the formation of substance-Mtd-P Related Polypeptide complexes and removing and/or detecting the complexes. The complexes can be detected by assaying for substance-Mtd-P Related Polypeptide complexes, for free substance, or for non-complexed Mtd-P Related Polypeptide. Conditions which permit the formation of substance-Mtd-P Related Polypeptide complexes may be selected having regard to factors such as the nature and amounts of the substance and the polypeptide.

The substance-protein complex, free substance or non-complexed polypeptides may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against Mtd-P Related Polypeptide or the substance, or labelled Mtd-P Related Polypeptide, or a labelled substance may be utilized. The antibodies, polypeptides, or substances may be labelled with a detectable substance as described above.

A Mtd-P Related Polypeptide, or the substance used in the method of the invention may be insolubilized. For example, a Mtd-P-Related Polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating a compound for its ability to modulate the biological activity of a Mtd-P Related Polypeptide of the invention, by assaying for an agonist or antagonist (i.e., enhancer or inhibitor) of the binding of a Mtd-P Related Polypeptide with a substance which binds with a Mtd-P Related Polypeptide. The basic method for evaluating if a compound is an agonist or antagonist of the binding of a Mtd-P Related Polypeptide and a substance that binds to the polypeptide, is to prepare a reaction mixture containing the Mtd-P Related Polypeptide and the substance under conditions which permit the formation of substance-Mtd-P Related Polypeptide complexes, in the presence of a test compound. The test compound may be initially added to the mixture, or may be added subsequent to the addition of the Mtd-P Related Polypeptide and substance. Control reaction mixtures without the test compound or with a placebo are also prepared. The formation of complexes is detected and the formation of complexes in the control reaction but not in the reaction mixture indicates that the test compound interferes with the interaction of the Mtd-P Related Polypeptide and substance. The reactions may be carried out in the liquid phase or the Mtd-P Related Polypeptide, substance, or test compound may be immobilized as described herein. The ability of a compound to modulate the biological activity of a Mtd-P Related Polypeptide of the invention may be tested by determining the biological effects on cells.

It will be understood that the agonists and antagonists, i.e., inhibitors and enhancers, that can be assayed using the methods of the invention may act on one or more of the binding sites on the polypeptide or substance including agonist binding sites, competitive antagonist binding sites, non-competitive antagonist binding sites or allosteric sites.

The invention also makes it possible to screen for antagonists that inhibit the effects of an agonist of the interaction of Mtd-P Related Polypeptide with a substance which is capable of binding to the Mtd-P Related Polypeptide. Thus, the invention may be used to assay for a compound that competes for the same binding site of a Mtd-P Related Polypeptide.

The invention also contemplates methods for identifying compounds that bind to proteins that interact with a Mtd polypeptide, in particular a Mtd-P Related Polypeptide. Protein-protein interactions may be identified using conventional methods such as co-immunoprecipitation, crosslinking and co-purification through gradients or chromatographic columns. Methods may also be employed that result in the simultaneous identification of genes which encode proteins interacting with a Mtd-P Related Polypeptide. These methods include probing expression libraries with labelled Mtd-P Related Polypeptide.

Two-hybrid systems may also be used to detect protein interactions in viva. Generally, plasmids are constructed that encode two hybrid proteins. A first hybrid protein consists of the DNA-binding domain of a transcription activator protein fused to a Mtd-P Related Polypeptide, and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the methods described herein In particular, Mtd-P Related Polypeptides fused to a glutathione-S-transferase may be used in the methods.

A modulator of a Mtd-P Related Polypeptide of the invention may also be identified based on its ability to inhibit or enhance activity of the polypeptide.

In aspects of the invention, substances that modulate trophoblast cell death, differentiation, and/or cell fusion and turnover and/or regulate trophoblast invasion can be selected by assaying for a substance that inhibits or stimulates the activity of a Mtd-P Related Polypeptide, in particular Mtd-P of SEQ ID NO. 1. Such a substance can be identified based on its ability to specifically interfere with or stimulate the activity of a Mtd-P Related Polypeptide, in particular Mtd-P of SEQ ID NO. 1, in assays and models such as those described herein.

Thus the invention contemplates a method for evaluating a compound for its ability to modulate trophoblast cell death, differentiation, cell fusion and turnover and/or regulate trophoblast invasion comprising the steps of:
(a) reacting a Mtd-P Related Polypeptide, in particular Mtd-P of SEQ ID NO. 1, or a part thereof, and a substance that binds to the polypeptide or part thereof, and a test agent, wherein the Mtd-P Related Polypeptide or part thereof forms a complex with the substance; and
(b) comparing to a control in the absence of the test agent to determine the effect of the substance.

The test agent may stimulate or inhibit the interaction of a Mtd-P Related Polypeptide, in particular Mtd-P of SEQ ID NO. 1 or a part thereof.

The invention also provides a method for assessing the potential efficacy of a test agent for treating a condition requiring modulation of trophoblast cell death, differentiation, cell fusion and turnover, and/or regulate trophoblast invasion in a patient, the method comprising comparing:
(a) levels of one or more Mtd-P Related Polypeptides, and/or polynucleotides encoding Mtd-P Related Polypeptides, and optionally other markers, in a first sample obtained from a patient and exposed to the test agent; and
(b) levels of one or more Mtd-P Related Polypeptides, and/or polynucleotides encoding Mtd-P Related Polypeptides, and optionally other markers, in a second sample obtained from the patient, wherein the sample is not exposed to the test agent, wherein a significant difference in the levels of expression of one or more Mtd-P Related Polypeptides, and/or polynucleotides encoding Mtd-P Related Polypeptides, and optionally the other markers, in the first sample, relative to the second sample, is an indication that the test agent is potentially efficacious for treating the condition in the patient.

The first and second samples may be portions of a single sample obtained from a patient or portions of pooled samples obtained from a patient.

In an aspect, the invention provides a method of selecting an agent for treating a condition requiring modulation of trophoblast cell death, differentiation, invasion, and/or cell fusion and turnover in a patient comprising:
(a) obtaining a sample from the patient;
(b) separately maintaining aliquots of the sample in the presence of a plurality of test agents;
(c) comparing one or more Mtd-P Related Polypeptides, and/or polynucleotides encoding Mtd-P Related Polypeptides and optionally other markers, in each of the aliquots; and
(d) selecting one of the test agents which alters the levels of one or more Mtd-P Related Polypeptides, and/or polynucleotides encoding Mtd-P Related Polypeptides and optionally other markers in the aliquot containing that test agent, relative to other test agents.

Further, the present invention provides a method of conducting a drug discovery business comprising:
(a) providing one or more methods or assay systems for identifying agents, compounds or inhibitors as described herein, in particular a method for identifying agents by their ability to modulate a Mtd-P Related Polypeptide or Mtd-Related Polynucleotide, and/or a condition mediated by a Mtd-P Related Polypeptide;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:
(a) providing one or more assay systems for identifying agents by their ability to modulate a Mtd-P Related Polypeptide or Mtd-Related Polynucleotide, and/or a condition mediated by a Mtd-P Related Polypeptide;
(b) (optionally) conducting therapeutic profiling of agents identified in step (a) for efficacy and toxicity in animals; and
(c) licensing, to a third party, the rights for further drug development and/or sales for agents identified in step (a), or analogs thereof.

The method may further comprise the steps of preparing a quantity of the agent and/or preparing a pharmaceutical composition comprising the agent.

The reagents suitable for applying the methods of the invention to evaluate compounds that modulate a Mtd-P Related Polypeptide or Mtd-P Related Polynucleotide may be packaged into convenient kits providing the necessary-materials packaged into suitable containers. The kits may also include suitable supports useful in performing the methods of the invention.

### 4.5 Compositions and Treatments

The polypeptides of the invention, substances, agents, or compounds identified by the methods described herein, antibodies, and polynucleotides of the invention may be used for modulating the biological activity of a Mtd-P Related Polypeptide, and they may be used in the treatment of conditions requiring regulation of trophoblast cell death, differentiation, invasion, and/or cell fusion and turnover such as preeclampsia in a patient.

The terms “subject”, “individual” or “patient” refer to a warm-blooded animal such as a mammal. In particular, the terms refer to a human. A subject, individual or patient may be afflicted with or suspected of having or being pre-disposed to a disease or a condition as described herein. The term also includes domestic animals bred for food or as pets, including horses, cows, sheep, poultry, fish, pigs, cats, dogs, and zoo animals. Methods herein for administering an agent or composition to subjects/individuals/patients contemplate treatment as well as prophylactic use. Typical subjects for treatment include persons susceptible to, suffering from or that have suffered a condition described herein. In particular, suitable subjects for treatment in accordance with the invention are persons that are susceptible to, suffering from or that have preeclampsia.

The agents/substances, antibodies, peptides, and compounds may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to living organisms including humans and animals. Administration of a therapeutically active amount of a pharmaceutical composition of the present invention is defined as an amount effective, at dosages and for periods of time necessary to achieve the desired result. For example, a therapeutically active amount of a substance may vary according to factors such as the disease state, age, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner including by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

The polynucleotides comprising full length cDNA sequences and/or their regulatory elements enable a skilled artisan to use sequences encoding a polypeptide of the invention as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is well known in the art, and sense or antisense oligomers, or larger fragments, can be designed from various locations along the coding or control regions.

Vectors derived from retroviruses, adenovirus, herpes or vaccinia viruses, or from various bacterial plasmids, may be used to deliver polynucleotides to a targeted organ, tissue, or cell population. Methods well known to those skilled in the art may be used to construct recombinant vectors which will express antisense polynucleotides of the invention. (See, for example, the techniques described in Sambrook et al (supra) and Ausubel et al (supra)).

Genes encoding a polypeptide of the invention can be turned off by transfecting a cell or tissue with vectors which express high levels of a desired Mtd-P-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a polypeptide of the invention, i.e., the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes (i.e., micRNA). Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a polypeptide of the invention.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

In aspects of the invention, methods are provided for modulating trophoblast cell death, differentiation, invasion, and/or cell fusion and turnover or regulating trophoblast invasion comprising directly or indirectly inhibiting or stimulating a Mtd Polypeptide including a Mtd-P Related Polypeptide, preferably inhibiting or stimulating a Mtd-P of SEQ ID NO. 1. In an embodiment of the invention, a method is provided for reducing trophoblast cell death, differentiation, invasion, and/or cell fusion and turnover in a subject comprising administering an effective amount of a substance which is an inhibitor of a Mtd-P Related Polypeptide, in particular Mtd-P of SEQ ID NO. 1. In particular, methods are provided for treating a women suffering from or who may be susceptible to preeclampsia.

In another embodiment of the invention, a method is providing for increasing trophoblast cell death, differentiation, invasion, and/or cell fusion and turnover in a subject comprising administering an effective amount of a Mtd-P Related Polypeptide, in particular Mtd-P of SEQ ID NO. 1 or a stimulator of same. The method may be used to monitor or treat choriocarcinoma or hydatiform mole.

The methods of the invention may also be used to monitor, treat, or prevent other complications of pregnancy such as intrauterine growth restriction, molar pregnancy, preterm labour, preterm birth, fetal anomalies, or placental abruption.

A substance that regulates trophoblast invasion may be a molecule which interferes with the transcription and/or translation of a Mtd Polypeptide, in particular a Mtd-P Related Polypeptide, more particularly Mtd-P of SEQ ID NO. 1. For example, the sequence of a nucleic acid molecule encoding a Mtd-P Related Polypeptide, in particular Mtd-P of SEQ ID NO. 1 or fragments thereof, may be inverted relative to its normal presentation for transcription to produce an antisense nucleic acid molecule. An antisense nucleic acid molecule may be constructed using chemical synthesis and enzymatic ligation reactions using procedures known in the art.

The treatment methods and compositions described herein may use substances that are known inhibitors of a Mtd Polypeptide.

The utility of a selected inhibitor or stimulator may be confirmed in experimental model systems. For example, the human villous explant culture system described by Genbacev et al. (Placenta. 1992 September-October; 13(5):439-61) or the methods described herein can be used to confirm the utility of an inhibitor for treatment of preeclampsia.

In a preferred embodiment of the invention a method is provided for treating a woman suffering from, or who may be suspectible to preeclampsia comprising administering therapeutically effective dosages of an inhibitor of a Mtd polypeptide, in particular a Mtd-P Related Polypeptide, more particularly Mtd-P of SEQ ID NO. 1, or a substance identified in accordance with the methods of the invention. Treatment with the inhibitor is discontinued after Mtd-P Related Polypeptide, in particular Mtd-P of SEQ ID NO. 1 levels are within normal range, and before any adverse effects of administration of the inhibitor are observed. Preferably treatment with the inhibitor begins early in the first trimester, and may continue until measured Mtd-P Related Polypeptide, in particular Mtd-P of SEQ ID NO. 1, levels are within the normal range. Preferably, treatment with the inhibitor or substance is not continued beyond about 30 weeks of gestation. For the purposes of the present invention normal levels of a Mtd-P Related Polypeptide, in particular Mtd-P of SEQ ID NO. 1 are defined as those levels typical for pregnant women who do not suffer from preeclampsia.

One or more inhibitors or one or more stimulators of a Mtd-P Related Polypeptide, in particular Mtd-P of SEQ ID NO. 1 or substances selected in accordance with the methods of the invention including binding agents, may be incorporated into a composition adapted for modulating trophoblast cell death, differentiation, invasion, and/or cell fusion and turnover. In an embodiment of the invention, a composition is provided for treating a woman suffering from, or who may be susceptible to preeclampsia, comprising a therapeutically effective amount of an inhibitor of a Mtd-P Related Polypeptide, in particular Mtd-P of SEQ ID NO. 1, or substance selected in accordance with the methods of the invention including antibodies or binding agents, and a carrier, diluent, or excipient.

A compositions of the invention can contain at least one inhibitor or stimulator of a Mtd-P Related Polypeptide, in particular Mtd-P of SEQ ID NO. 1, or substance identified in accordance with the methods of the invention, alone or together with other active substances.

The compositions of the invention may be administered together with or prior to administration of other biological factors that have been found to affect trophoblast proliferation. Examples of these factors include IL-11 (Ireland et al Blood 84:267a. 1994), G-CSF, GM-CSF and M-CSF (U.S. Pat. No. 5,580,554 to Keith).

A composition of the invention contains a therapeutically effective dose of an inhibitor, for example, an amount sufficient to lower levels of Mtd-P Related Polypeptide to normal levels is about 1 to 200 μg/kg/day. A method of the invention for modulating trophoblast cell death, differentiation, invasion, and/or cell fusion and turnover may involve a series of administrations of the composition. Such a series may take place over a period of 7 to about 21 days and one or more series may be administered. The composition may be administered initially at the low end of the dosage range and the dose will be increased incrementally over a preselected time course.

An inhibitor or stimulator of a Mtd-P Related Polypeptide, in particular Mtd-P of SEQ ID NO. 1, or a substance identified in accordance with the methods of the invention may be administered by gene therapy techniques using genetically modified trophoblasts or by directly introducing genes encoding the inhibitors or stimulators into trophoblasts in vivo. Trophoblasts may be transformed or transfected with a recombinant vector (e.g. retroviral vectors, adenoviral vectors and DNA virus vectors). Genes encoding inhibitors or stimulators, or substances may be introduced into cells of a subject in vivo using physical techniques such as microinjection and electroporation or chemical methods such as coprecipitation and incorporation of DNA into liposomes. Antisense molecules may also be introduced in vivo using these conventional methods.

### 4.6 Other Applications

The polynucleotides disclosed herein may also be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a polypeptide of the invention. Cells, tissues, and non-human animals lacking in expression or partially lacking in expression of a polynucleotide or gene of the invention may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting, or by assaying for expression of the encoded polypeptide using the methods described herein. Such cells may then be fused to embryonic stem cells to generate transgenic non-human animals deficient in a polypeptide of the invention. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a gene encoding a Mtd-P Related Polypeptide. In an embodiment the invention provides a transgenic non-human mammal all of whose germ cells and somatic cells contain a recombinant expression vector that inactivates or alters a gene encoding a Mtd-P Related Polypeptide resulting in a Mtd-P Related Polypeptide associated pathology. Further, the invention provides a transgenic non-human mammal which does not express or has altered (e.g., reduced) expression of a Mtd-P Related Polypeptide of the invention.

The invention also provides a transgenic non-human animal assay system which provides a model system for testing for an agent that reduces or inhibits a pathology associated with a Mtd-P or Mtd-P Related Polypeptide, preferably preeclampsia, comprising:
(a) administering the agent to a transgenic non-human animal of the invention; and
(b) determining whether said agent reduces or inhibits the pathology (e.g., Mtd-P Related Polypeptide associated pathology) in the transgenic non-human animal relative to a transgenic non-human animal of step (a) which has not been administered the agent.

The agent may be useful in the treatment and prophylaxis of conditions such as preeclampsia as discussed herein. The agents may also be incorporated in a pharmaceutical composition as described herein.

The following non-limiting examples are illustrative of the present invention:

### Example 1

The following materials and methods were used in the study described in the example.

### Materials and Methods

### Tissue Collection

Collection was in accordance with participating institutions' ethics guidelines. First-trimester human placental tissues (5-13 weeks of gestation, n=30) were obtained from elective terminations of pregnancies by dilatation and curettage. Preeclamptic group was selected based on ACOG clinical and pathological criteria (43). Calcified, necrotic and visually ischemic areas were omitted from sampling. Patients with diabetes, infections and kidney disease were excluded. Pregnant patients with essential hypertension (EH; n=4, at term) and pregnancies affected by intrauterine growth restriction (IUGR; n=6, gestational age 32-36 weeks with fetal weight less than 5th %) without preeclampsia were included as controls. Preterm control patients did not show signs of preeclampsia or other placental disease. Preterm deliveries were due to multiple pregnancy (27%), preterm labour due to incompetent cervix (41%), premature preterm rupture of membrane (18%) and spontaneous rupture of membranes (14%). Clinical data is summarized in Table 1.

### Human Chorionic Villous Explant Culture

Explant cultures were performed as previously described (27). Explants were maintained in standard condition (5% CO₂ in 95% air) or in an atmosphere of 3% O₂/92% N₂/5% CO₂ for 48 hrs at 37° C. In separate experiments hypoxia/re-oxygenation was performed as previously described (20) from 20% O₂ to low oxygen condition: 2-3% O₂ to re-oxygenation 20% O₂.

### RNA Analysis and Targeting (Antisense Knockdown)

RNA was extracted using a Rneasy Mini Kit (Qiagen), random hexamer reverse transcribed, and amplified by 20 cycles of PCR (5 minutes at 95° C., cycle: 30 seconds at 95° C., 30 seconds at 55° C. and 1.5 minutes at 72° C.). Mtd (NM_{—}032515): (forward) 5′-ATCCTGAAGCCAGAACTCCA-3′, (reverse) 5′-AAGATGTGTTCGGGTGCTGA-3′ (predicted sizes: Mtd-L: 794 bp, Mtd-S: 665 bp); β-actin (NM_{—}001101): (forward) 5′-CTTCTACAATGAGCTGCGTG-3′, (reverse) 5′-TCATGAGGTAGTCAGTCAGG-3′ (predicted size=304 bp). Products were confirmed by sequencing. No signal was detected without addition of reverse transcriptase. RT-PCR products were analyzed by Southern blotting using Mtd and β-actin cDNAs labeled with α-32P-dCTP (PerkinElmer Life Sciences), using a random hexamer approach. Quantitative PCR was performed using the SYBR Green I dye DyNamo™ HS kit (MJ Research) based on the manufacturer's protocol using isoform specific primers (Mtd-L: Forward 5′-GCCTGGCTGAGGTGTGC-3′, Mtd-P: Forward 5′-GCGGGAGAGGCGATGA, Reverse (both L and P) 5′-TGCAGAGAAGATGTGGCCA-3′). Analysis was done using the DNA Engine Opticon®2 System (MJ Research). Data were normalized against expression of 18S ribosomal RNA as previously described (44). Mtd knockdown was performed in villous explants using phosphorothioated (all positions) sense and antisense oligos designed against Mtd transcript NM_{—}032515 at a final concentration of 10 μM (L-Sense: 5′-CATGGAGGTGCTGCGG-3′, LAntisense: 5′-CCGCAGCACCTCCATG-3′, P-Sense: 5′-AGGCGATGAGCTGGAGATGA-3′, PAntisense: 5′-TCATCTCCAGCTCATCGCCT-3′).

### Western Blot Analysis

Western blot analyses were performed as previously described (45). Primary Antibodies (1:1000): rabbit polyclonal Mtd antibody (generous gift of Dr. J. Tilly) (46), or cleaved caspase-3 rabbit polyclonal antibody (Cell Signaling). For Mtd antibody, pre-immune serum was used as control. Secondary antibody (1:5000): horseradish peroxidase-conjugated anti-rabbit (Santa Cruz Biotechnology). All immunoblots were checked for equivalent protein loading using ponceau staining.

### Immunohistochemistry

Immunohistochemical analyses were performed as previously described (27,45). Primary antibody: rabbit polyclonal anti-Mtd (1:200) or mouse monoclonal anti-ssDNA (1:500), the latter used based on manufacturer's protocol (MAB3299, Chemicon). Secondary antibody (1:400): biotinylated goat anti-rabbit or goat anti-mouse IgG (Vector Laboratories).

### TUNEL (Terminal Deoxynucleotidyl Transferase-dUTP-Nick End Labelling)

The in situ Cell Death Detection kit (Roche Molecular Biochemicals) was used based on the manufacturer's protocol.

### Plasmid Construction

Open reading frames (ORF) of Mtd-L and Mtd-P were directional cloned in pcDNA3.1/Hygro(+) (Invitrogen). Forward and reverse primers used encoded a KpnI and a BamHI restriction site respectively. Mtd-P primers: (Forward: 5′-CCCGGTACCACCATGATCCGGC CCAGCGTCTAC-3′, Reverse: 5′-CCCGGATCCGGGTCATCTCTCTGGCAGCAGCAC-3′).

Mtd-L primers (Forward: 5′-CCCGGTACCACCATGATCCGGCCCAGCGTCTAC-3′, Reverse: 5′-CCCGGATCCGGGTCATCTCTCTGGCAACAACAGGAA-3′).

### Transfection Studies

Cell culture (CHO: Chinese hamster ovary cells and BeWo: Human choriocarcinoma cytotrophoblast cells, ATCC), transfection, fixation, P-gal staining were performed as previously described (17). BeWo cells were cultured based on the manufacturer's protocol (ATCC). Transfection was performed with 1.5 μg of construct per 35-mm plate (empty pcDNA3.1/Hygro(+), Mtd-P or Mtd-L in pcDNA3.1/Hygro(+) plasmid) using lipofectamine reagent (Invitrogen) in combination with 0.1 μg fraction of pcDNA3.1/Hygro(+) encoding the lacZ gene based on manufacturer's recommendations.

### DNA Laddering

Genomic DNA was extracted 24 hours post-transfection as previously described (47). Samples were separated on 2% (wt/vol) agarose gel containing 0.5 μg/ml ethidium bromide for 2 hours at 40 volts and visualized using a UV transluminator.

### Mitochondrial Membrane Potential Analysis

Membrane potential was assessed by 5,5′,6,6′-tetrachloro-1,1′,3,3′-tetraethylbenzimidazolcarbocyanine iodide (JC-1,3 μg/ml working concentration; Molecular Probes) staining based an the manufacturer's protocol for staining in culture dish and FACS analysis. Images were captured using an inverted fluorescent microscope (Leica DMIRB) equipped with fluorescein and rhodamine filters. Stained CHO cells (live cells, dead cells (floating fraction of serum-starved cells) and transfected conditions) were subjected to FACS analysis on an EPICS Elite (Beckman-Coulter) using green (JC-1 monomers) and red (JC-1 aggregates) fluorescence signals resolved by detection in conventional FL1 and FL2 channels, respectively.

### Statistical Analysis

Data are represented as mean±SEM of at least 3 separate experiments carried out in triplicate. Statistical difference was determined by Student's t test for paired groups, Significance defined as P<0.05.

### Results

### Mtd Gene Expression in First-Trimester Placental Tissues

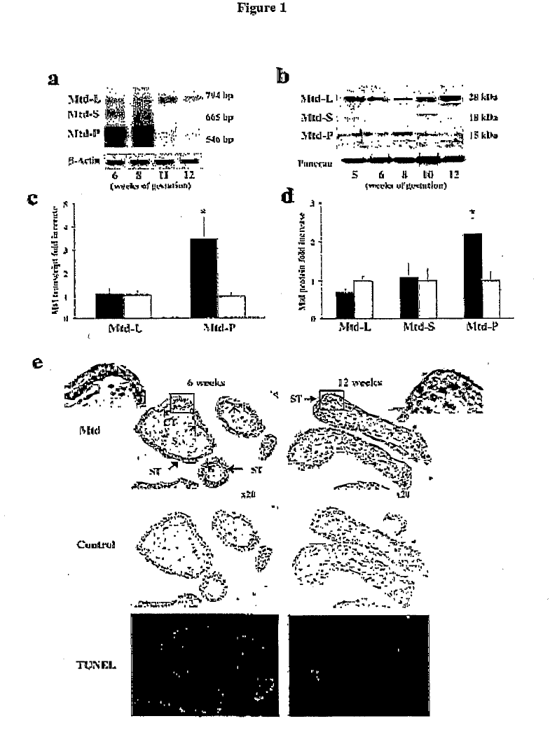

The expression of previously characterized transcripts of Mtd (Mtd-L and Mtd-S) was observed in first-trimester tissues (6-12 weeks) (FIG. 1a). While Mtd-L expression (794 bp) was constant throughout first-trimester samples, Mtd-S expression (665 bp) appeared to decrease in tissues from 10-12 weeks when compared to earlier gestations. Importantly, a 546 bp band exhibiting strong Mtd-specific hybridization was observed predominantly around 6-8 weeks. Sequencing of the 546 bp band revealed a novel-Mtd transcript referred to herein as Mtd-P.

### Mtd-P is a Novel Mtd Transcript Resulting From Exon II Skipping

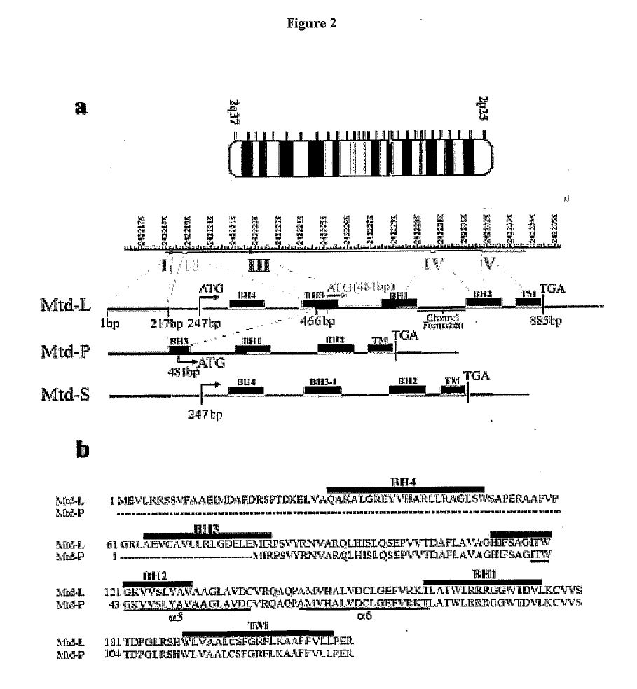

Sequence analysis of Mtd-P transcript showed a 249 bp deletion spanning bp 217 to 466 in the full-length mRNA (NM_{—}032515). This deletion included part of the 5′ UTR, the original start methionine, the BH4 and part of the BH3 domain, resulting in a deletion of 83 amino acids (FIG. 2a). The genomic structure of Mtd is composed of five putative exons and four introns localized on chromosome 2q37.3. Mtd-P was noted to undergo exon II skipping (orange box), resulting in deletion of BH4 and part of BH3 domains. The protein product of Mtd-P has a theoretical molecular weight of approximately 15 kDa (FIG. 2b). The putative pore-forming region encoded by exon IV is retained in all isoforms.

### Mtd Protein Expression in First-Trimester Placental Tissues

Mtd protein expression was examined using an antibody generated against a peptide sequence derived between BH2 and BH1 domains of Mtd-L, thus recognizing all known isoforms. Immunoblotting confirmed expression of previously characterized Mtd isoforms (Mtd-L and S) in first-trimester tissues in addition to a novel Mtd-specific band with an apparent molecular weight of 15 kDa, corresponding to the predicted molecular weight of Mtd-P (FIG. 1b). Mtd-S runs at its predicted molecular weight (18 kDa), while Mtd-L (predicted molecular weight: 23 kDa) was observed at an apparent molecular weight of 28 kDa, possibly due to post-translational modifications (FIG. 1b). While Mtd-L and Mtd-S protein expression was constant across 1^{st} trimester gestations based on Western blotting and densitometric analyses, Mtd-P was the only isoform whose expression significantly increased around 6-8 weeks when compared to later gestations (Mtd-P 2.2 fold increase, p=0.04) (FIG. 1d). Similar to protein expression, quantitative real-time PCR demonstrated that while Mtd-P transcript expression increased significantly in early first-trimester compared to later gestations (3.4 fold increase, P=0.03), Mtd-L gene expression was unchanged across 1st trimester samples (FIG. 1c).

Mtd spatial localization was assessed by immunohistochemistry. Positive immunoreactivity was mostly observed in early first-trimester sections (6 weeks) and was predominately localized to the progenitor mononucleated cytotrophoblast (CT) cells (FIG. 1e, left panel). Low/absent Mtd immunoreactivity was noted in the multinucleated syncytiotrophoblast layer (ST). In contrast, tissue sections from later first-trimester gestations (12 weeks) exhibited low/absent Mtd expression in CT cells (right panel) while Mtd positive immunoreactivity was detected in the apical membrane of the syncytiotrophoblast layer. Stromal regions were Mtd negative. Neighboring control sections (no 1° Ab) were also Mtd negative. Finally, Mtd expression co-localized with TUNEL staining also predominantly observed in early 1st trimester compared to later gestations (lower panels).

### Mtd Expression is Elevated in Preeclampsia

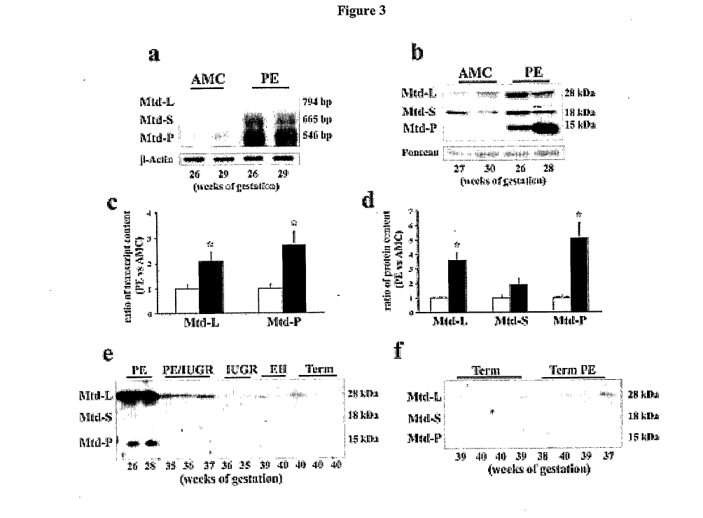

As preeclampsia is characterized by increased trophoblast cell death, whether Mtd expression was altered in placentae complicated by this disease was next examined. Mtd gene expression was increased in preeclamptic placental tissues particularly with respect to Mtd-P when compared to normotensive age-matched controls (FIG. 3a). Densitometric analyses (RT-PCR followed by Southern blot) revealed that elevated gene expression was only significant with respect to Mtd-L and Mtd-P transcripts in preeclamptic samples, but not Mtd-S (Mtd-L: 2.7-fold P=0.004, Mtd-S: 1.7-fold (not significant) and Mtd-P: 3.5-fold P=0.006). qRT-PCR analyses further validated that the expression Mtd-L and particularly that of Mtd-P increased in preeclamptic tissues when compared to preterm age-matched normotensive control patients (Mtd-L 2.1-fold p=0.03 and Mtd-P 3.3 fold P=0.01, FIG. 3c). Increased Mtd protein content in early onset preeclampsia was particularly notable with respect to Mtd-P (FIG. 3b). Protein densitometric analysis revealed that Mtd-L and Mtd-P were significantly elevated in preeclamptic tissues by approximately 3.6-fold (P<0.05) and 5.1-fold (P<0.05) respectively, when compared to preterm age-matched control tissues (FIG. 3d). Similar to Mtd-S gene expression, the 1.9-fold increase in Mtd-S protein level in preeclampsia was not statistically significant when compared to controls (FIG. 3d).

Mtd protein expression was compared between preeclamptic pregnancies (early and late onset) and normotensive pregnancies affected by IUGR, essential hypertension as well as normal term deliveries. Only preeclamptic placentae complicated by early severe onset of disease exhibited increased expression of Mtd-L and particularly that of Mtd-P molecule (FIG. 3e). Normal term placentae, IUGR placentae and placentae from pregnancies from essential hypertensive subjects did not show increased Mtd expression. Interestingly, tissues from later third trimester patients (35-37 weeks) affected by preeclampsia in combination with IUGR showed increased expression of Mtd-L, but not Mtd-P, when compared to control subjects. Additionally, no differences in Mtd expression were observed between term (late onset) preeclamptic tissues and appropriate controls (FIG. 3f).

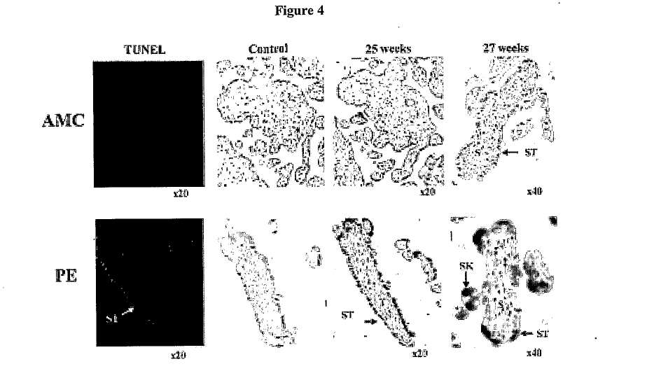

Immunohistochemical analysis demonstrated strong positive Mtd immunoreactivity in all trophoblast cell layers of early onset preeclamptic placental tissues when compared to controls (FIG. 4). Interestingly, in preeclamptic tissues, increased Mtd expression was localized to syncytial knots (SK) demonstrating the potential involvement of this molecule in increased trophoblast cell death leading to increased shedding of STBMs in preeclampsia (lower right panel). In contrast, low/absent Mtd expression was observed in placental sections of normotensive age-matched controls (upper panels). TUNEL analysis demonstrated a notable increase in apoptotic cell death in the placental trophoblast cell layers of preeclamptic tissues when compared to age-matched control samples.

### Mtd-P is a Pro-Apoptotic Molecule Exerting its Function Through the Mitochondrial Pathway

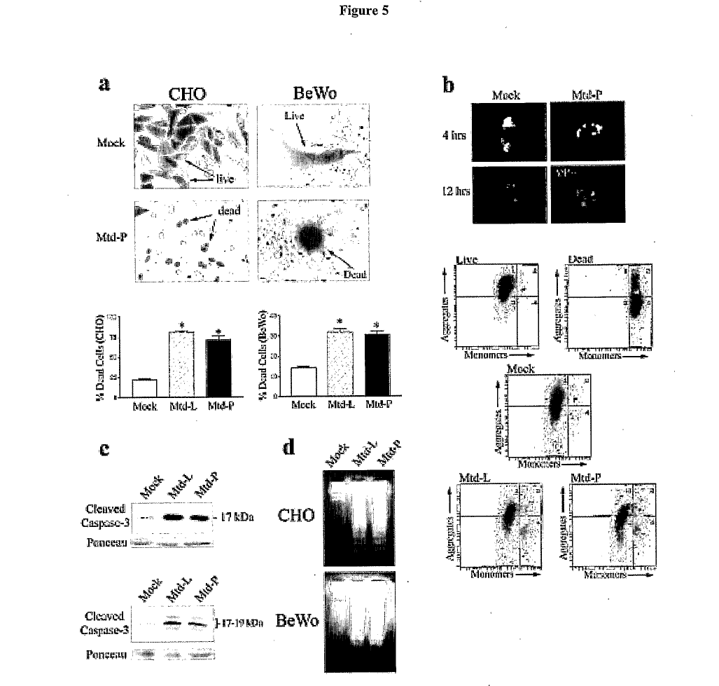

The apoptotic function of Mtd-P was next assessed. Mtd-P as well as Mtd-L (positive control) over-expression in hamster ovary-derived CHO cells and human cytotrophoblast-derived BeWo cells resulted in rounding-up of cells into apoptotic bodies (FIG. 5a, arrows). Mtd-P overexpression in CHO and BeWo cells resulted in significant increased cell death within 24 hours post-transfection when compared to cells transfected with the empty vector DNA (FIG. 5a). Cell death induced by Mtd-P was similar to that induced by Mtd-L in CHO and BeWo cells (FIG. 5a).

Whether cell death was induced via a mitochondrial pathway was examined. The JC-1 dye, a cationic carbocyanine fluorescent molecule with dual emission properties, was used to monitor mitochondrial membrane potential in CHO cells for up to 12 hours post-transfection and preceding the morphological signs of apoptosis. Mitochondrial depolarization in Mtd-P transfection (same cell 4 hours and 12 hours post-transfection) caused a leakage of J-aggregates (red-orange color [590 nm]) from the mitochondria into the cytoplasm leading to JC-1 monomerization (greenish color [525 mm]) when compared to empty vector transfected cells (FIG. 5b). JC-1-labeled FACS analyses of Mock-transfected cells exhibited a similar population scattering pattern as untreated live cells (FIG. 5b low panels). Transfection alone resulted in a slight loss of red fluorescence in a live population of mock cells (quadrant 1) when compared to untreated live cells. Mtd-L and Mtd-P expressing cells exhibited a fluorescence shift in a subpopulation of cells from red (JC-1 aggregates, quadrant 1) to green (JC-1 monomers, quadrant 2 and 4). The percent of total cell population in quadrants 2 plus 4 (dead/dying cells) in mock, Mtd-L and Mtd-P transfection was respectively 4.6%±0.3, 24.35%±0.45 and 21.25%±0.35, demonstrating a significant increase (>5-fold, p<0.0001 both isoforms) in populations of dead/dying cells in Mtd-L and Mtd-P transfections when compared to mock.

Mitochondrial depolarization likely leads to the cytoplasmic release of apoptogenic factors and the activation of the caspase pathway. This was confirmed by increased cleavage of caspase-3 (17 kDa fragment in CHO cells and 17-19 kDa fragments in BeWo cells, the latter fragment pattern is typical of human cells), an executioner of the apoptotic pathway (FIG. 5c). Moreover, Mtd-P as well as Mtd-L transfections in either CHO or BeWo cells resulted in nuclear DNA laddering while mock transfected cells did not exhibit signs of internucleosomal DNA fragmentation (FIG. 5d).

### Mtd Expression is Increased Under Conditions of Low Oxygen/Oxidative Stress and Mediates Trophoblast Apoptosis

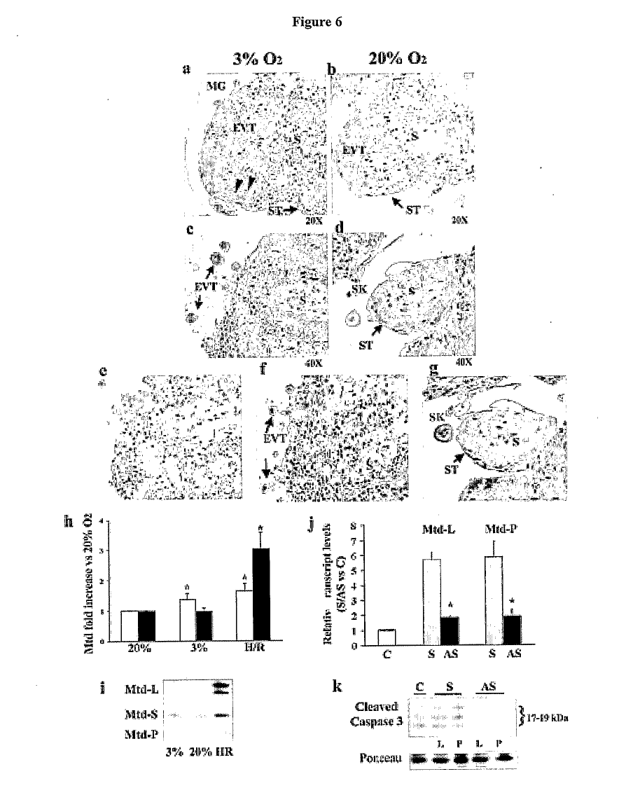

Elevated Mtd expression in early first-trimester placental tissues (5-8 weeks), (when placentation takes place in a low oxygenated environment) as well as the increased expression in preeclampsia, a disease characterize by placental hypoxia, led to the hypothesis that Mtd expression would be affected under conditions of reduced oxygenation. Therefore, the effect of reduced oxygen was investigated in vitro on Mtd protein expression in first-trimester placental villous explants. Immunohistochemical analysis of explants incubated under 3% and 20% oxygen demonstrated increased Mtd protein expression under 3% oxygen (FIG. 6a,c) when compared with standard conditions (20% O₂) (FIG. 6b,d). Immunoreactivity was predominantly localized to the low oxygen-induced extravillous trophoblast outgrowth region (EVT) as well as the CT cell layers of explants incubated under 3% O₂ (FIG. 6a,c). Low Mtd-positive immunostaining was also observed in ST cells. Stromal regions (under 3% or 20% O₂) were Mtd negative. Neighboring sections to those used for Mtd staining (FIG. 6c,d) were also probed with an anti-ssDNA antibody (FIG. 6e,f,g), which identifies single stranded DNA characteristic of apoptotic cells. It was observed that under 3% O₂ when compared to standard conditions, increased Mtd expression co-localized with active apoptosis in the same cells, demonstrating the involvement of Mtd in trophoblast cell death under conditions of reduced oxygenation. To determine which Mtd isoform (Mtd-L or Mtd-P) was induced under low oxygen tension, qRT-PCR was performed. Mtd-P gene expression, but not Mtd-L, was observed to significantly increase by 1.4-fold (p<0.05) under reduced oxygenation (3%) when compared to standard conditions (FIG. 6h). Similar to the transcript expression, Western blot analysis of Mtd further confirmed increased Mtd-P protein expression under conditions of reduced oxygenation relative to standard oxygenation (FIG. 6i). In contrast, Mtd-L and Mtd-S protein expressions remained unchanged between 3% and 20% oxygen (FIG. 6i). Additionally, as hypoxia/reoxygenation was recently demonstrated to be a potent inducer of trophoblast cell death (20), whether Mtd expression was altered under such conditions was tested. The transcript expressions of Mtd-L (3-fold, P=0.0007) and Mtd-P (1.6-fold, P<0.05) were significantly increased under H/R conditions when compared to standard conditions (FIG. 6h). Similarly, Mtd-L and Mtd-P protein expressions were also observed to increase in HR conditions relative to standard conditions (FIG. 6i). In summary, these data collectively demonstrate a direct correlative expression between the transcript and protein levels of Mtd isoforms in various oxygenation conditions.

To assess whether Mtd has a direct involvement in trophoblast cell death, the effect of Mtd knockdown was tested, using a previously validated antisense (AS) approach (21), in human villous explants and monitored for signs of apoptosis. The effect of AS isoform specific oligonucleotide treatment (AS-L and AS-P) was assessed under conditions of oxidative stress (HR) as this condition was shown to be the strongest inducer of Mtd isoforms. To confirm the success of Mtd transcript knockdown, Mtd-L and Mtd-P expressions where measured using qRT-PCR in control S and AS-treated conditions relative to untreated tissues (C). As previously observed, HR conditions significantly increased Mtd-L and Mtd-P transcript levels relative to control conditions (FIG. 6j). Furthermore, AS-Mtd-L and AS-Mtd-P treatments significantly decreased their expression levels by 68% and 67%, respectively, when compared to control sense-treated conditions (FIG. 6j). As cleaved caspase-3 expression has previously been demonstrated to be a reliable marker to trophoblast apoptosis (20), its expression was assessed by immunoblotting in explants treated with AS-Mtd (L and P) as well as control conditions (S-Mtd-L/P and no oligos: C). Antisense treatment resulted in markedly reduced cleavage of caspase-3 relative to control conditions demonstrating decreased levels of apoptosis (FIG. 6k).

### Discussion

This study reports the placental expression of two previously characterized Mtd isoforms (Mtd-L and Mtd-S) and most importantly the identification and characterization of Mtd-P, a novel Mtd splice variant resulting from exon II skipping. The data demonstrate that 1) Mtd-P has a distinctive developmental expression profile, 2) Mtd-P over-expression is unique to pregnancies complicated by severe early onset preeclampsia, 3) Mtd-P is a pro-apoptotic molecule involved in trophoblast cell death, and, 4) Mtd-P expression is increased under conditions of reduced oxygenation and oxidative stress.

Interaction between pro- and anti-apoptotic Bcl-2 family members is critical in the physiologic fine-tuning of apoptosis (11). The pro-apoptotic activity of the widely expressed Bax and Bak molecules is countered by their ability to interact with various anti-apoptotic Bcl-2 family members, including Bcl-2, Bcl-w and Bcl-xL (11). This contrasts the interaction capability of Mtd-L, which essentially heterodimerizes with the anti-apoptotic Mcl-1 molecule (17,19). Interestingly, Mtd-S, resulting from fusion of BH3 and BH1 domains, lacks the ability to interact with any presently known anti-apoptotic Bcl-2 family members (19). Mtd-P identified herein is missing 15 of the 18 amino acids that compose the BH3 domain. Mutation of key residues in the BH3 domain of Mtd-L as well as artificial fusion of BH3 and BH1 domains in Bax and Bak have no effect on the killing ability of these molecules (19). As well, site-directed mutagenesis of conserved BH3 residues LLRLGDEL to either glycine or alanine in Mtd-L abolishes the heteromerization properties of this protein with Mcl-1, but does not impede its killing ability (19). BH3 residues mutatedin Mtd-L are not present in the open reading frame of Mtd-P, suggesting that this molecule may induce apoptosis irrespective of its interaction with Mcl-1. It should also be noted that the start methionine in Mtd-P corresponds to a glutamine residue in rat and murine protein sequences, and to a tyrosine or arginine in chicken and Drosophila sequences, respectively (22), demonstrating the species specificity of Mtd-P.

Mitochondrial depolarization in Mtd-P transfected cells revealed important insights into its killing mechanism. The BH3 domain in the multi-domain subfamily of pro-apoptotic molecules (Bok/Mtd, Bax and Bak) seems irrelevant for killing, but rather the pore-forming region consisting of the α5 and α6 helixes between the BH2 and BH1 domains and to a lesser extent the transmembrane domain appear essential for inducing cell death via mitochondrial membrane destabilization. In support of this argument, a mutant Mtd protein lacking the BH1, BH2 and COOH-terminal hydrophobic tail domain was unable to induce apoptosis (18). Additionally, an artificially truncated version of Mtd-L only encompassing the BH4 and BH3 domains was also incapable of inducing cell death (22). Moreover, a recently identified chicken isoform of Mtd (MtdΔTM) lacking the transmembrane domain was also found to adversely affect mitochondrial function and sensitize transfected lymphoma-derived cells to apoptotic stimuli (23), once again stressing the importance of the pore-forming region in inducing cell death.

Increased Mtd-P expression in preeclampsia may be causative for increased trophoblast cell death and shedding observed in this disease (7,9,14-16,24,25). This is the first evidence of an expressional difference of a pro-apoptotic Bcl-2 family member in placentae of preeclamptic versus age-match control subjects. Previous studies have demonstrated expressional differences of other apoptotic-related molecules in preeclamptic and control subjects including elevated expression of serum Fas and elevated placental expression of FasL and p53 in preeclamptic placentae (15,26). Of clinical importance, Mtd-P increased expression in preeclamptic tissues appears unique to the early severe onset form of preeclampsia as tissues obtained from normotentive age-matched and term control subjects as well as term preeclampsia, IUGR and essential hypertensive subjects do not exhibit elevated Mtd-P expression. This may hence be a distinctive feature of placentae from patients suffering from severe early onset preeclampsia.

The findings of increased Mtd expression in vivo at 5-8 weeks of gestation, when placental pO₂ is low, and in vitro, in villous explants kept at either 3% O₂, is consistent with the idea of oxygen regulating placental Mtd expression and increased apoptosis during early first trimester placentation. Previous studies have demonstrated that, reduced oxygenation in vitro leads to increased levels of trophoblast proliferation (27-29). Therefore, it is plausible that mechanisms regulating trophoblast apoptosis and proliferation may be tightly intertwined and as such may both be affected by oxygen. Interestingly, Bcl-W, a pro-survival factor, was recently demonstrated to be involved in the regulation of cell cycle progression in spermatogenesis (30). BclxL/S and Bcl-2 have also been shown to enhance the potential for cellular differentiation by delaying entry into cell cycle (31-34). Studies from others have also linked Bag-1, anantiapoptotic molecule which interacts with Bcl-2 and a 70 kDa heat shock protein, to a number of cellular processes besides apoptosis, including cell signaling, proliferation, transcription and cell motility (30,35). Altogether these findings are indicative that members of the Bcl-2 family may play a central role in the regulation of global cellular processes other than apoptosis, possibly via yet undiscovered pathways involved in cell fate decisions.

What is particularly important in the findings is the increased expression of Mtd under conditions of low oxygen and hypoxia-reoxygenation. Previous studies have demonstrated that under hypoxia, primary cytotrophoblast cells, particularly populations isolated from preeclamptic placentae, exhibit increased levels of apoptosis (25,36). Importantly, reduced uteroplacental oxygenation and hypoxia-reoxygenation, a direct cause of oxidative stress as it may be the case in early severe onset preeclampsia, are potent inducers of villous trophoblast cell death (20). Using antisense knockdown, the studies described herein demonstrated that Mtd is a direct regulator of trophoblast cell death under conditions of oxidative stress. Moreover, it was shown that increased expression of Mtd-P as well as Mtd-L in trophoblast cells results in increased apoptotic cell death as measured by caspase-3 cleavage and internucleosomal DNA fragmentation.

Since several putative hypoxia-responsive elements are present in the human Mtd promoter region, it is hence plausible that Mtd expression is under HIF-1 (a transcriptional regulator of oxygen-responsive genes) control. Studies have demonstrated that HIF-1α, the oxygen labile-moiety of HIF-1 is elevated in preeclamptic placentae (37,38). Recent studies have demonstrated that expression of other pro-apoptotic Bcl-2 family members (Nix and Nip3) is directly regulated by HIF-1α under conditions of reduced oxygenation (39,40). Interestingly, hypoxia has also been shown to increase the expression of the pro-apoptotic Bax and decrease the expression of the anti-apoptotic Bcl-2 molecule in human trophoblast cells (36). Similar to what is demonstrated herein, these studies are supporting evidence that oxygen plays a key role in regulating the expression of molecules within the Bcl-2 family and highlight the importance of the mitochondrial pathway in regulating low-oxygen induced apoptosis. Increased Mtd expression in low pO₂/or oxidative stress may be directly regulated by HIF-1α or potentially other hypoxia-induced transcription factors such as NFκB and AP-1. Finally, increased Mtd expression in preeclampsia may also be p53-dependent as this transcription factor (a known regulator of Mtd) is increased in hypoxic/oxidative stress conditions as well as in preeclampsia and IUGR (15,16,36,41,42).

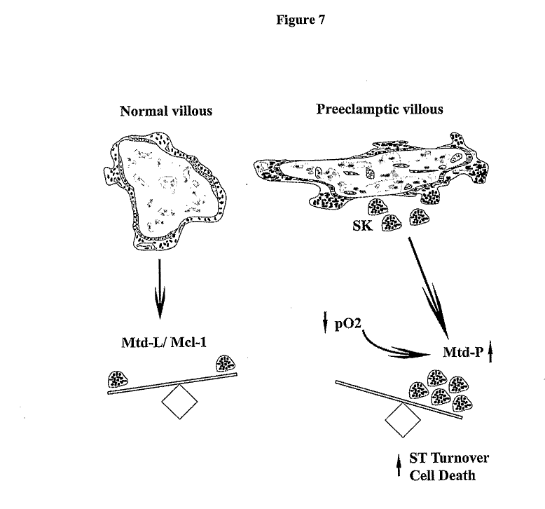

Mtd over-expression in preeclampsia, following reduced oxygenation and/or oxidative stress, may shift the intrinsic trophoblast apoptotic rheostat towards a death pathway and result in increased release of trophoblast microfragments in the maternal circulation (FIG. 7).

### Example 2

The Role of Mtd/Bok in Trophoblast Proliferation During Placental Development

Regulation of trophoblast cell proliferation during the first trimester is critical for proper placental function. Abnormal levels of proliferation have been linked to disease states such as pre-eclampsia and molar pregnancies. Here the involvement of a pro-apoptotic member of the Bcl-2 family, Mtd/Bok, is investigated in trophoblast cell cycle regulation. The objective of the study was to investigate the temporal and spatial pattern of expression of Mtd during the first trimester in human placenta and examine whether changes in Mtd expression were associated with specific cellular events.

Methods: Spatial and temporal location of Mtd was determined using fluorescence immunohistochemistry. First trimester placental sections were double stained using a polyclonal antibody that recognizes all isoforms of Mtd in conjunction with markers of either proliferation (ki67 and PCNA), or cell death (casp-3), to enable association of Mtd expression with cellular fate.
Results: The data obtained showed a strong co-localization of Mtd with markers of proliferation (Ki67 and PCNA) in both chorionic villous “stem” cytotrophoblast cells as well as in extra villous trophoblast cells forming the proximal region of the anchoring villi. An increased number of cells were Mtd positive in placenta samples from 5-7 wks when cell death in the placenta is low and proliferation was high and were seen to decrease in samples from later in the first trimester (9-15 wks), when apoptosis increases and proliferation decreases.
Conclusion: The data suggests that Mtd not only regulates cell death, as previously determined, but may also be involved in regulating the cell cycle of trophoblast cells during early placental development.

### Example 3

### Caspase Activation Regulates Stability of the Myeloid Cell Leukemia (Mcl-1) Protein Thereby Modulating Trophoblast Cell Death in Preeclampsia

Placentae from pregnancies complicated by preeclampsia exhibit increased trophoblast cell death, a process that is believed to increase syncytiotrophoblast microfragment shedding into the maternal circulation. Excess trophoblast turnover may be responsible for the generation of maternal endothelial cell damage. Preeclamptic placentae also exhibit reduced uteroplacental perfusion resulting in placental hypoxia and oxidative stress. Members of the Bcl-2 family are important intrinsic regulators of apoptosis in normal development as well as in diseases. The Mcl-1 gene, a member of the Bcl-2 family, is composed of three exons and functions as an important anti-apoptotic molecule (Yang et al. J Cell Biol. 1995 March, 128(6):1173-84). In addition to the anti-apoptotic Mcl-1L molecule, the Mcl-1 gene also gives rise to an alternate transcript known as Mcl-1S, resulting from exon II skipping (Bae et al., J Biol Chem. 2000 Aug. 18; 275(33):25255-61; Bingle et al., J Biol Chem. 2000 Jul. 21; 275(29):22136-46). Exon II skipping in Mcl-1S generates a truncated pro-apoptotic “BH3-only” containing protein. Mcl-1L is the only known molecule that can bind to and antagonize the killing capability of Mcl-1S (Bae et al., 2000). In recent years, the protein stability of Mcl-1 has become the subject of intense interest. Studies have demonstrated that caspase-3 activation results in proteolytic cleavage of conserved aspartate residues (D127 and D157) within the PEST sequence of Mcl-1L and Mcl-1S proteins, hence generating N-terminal and C-terminal truncated molecules (Han et al. J Biol Chem. 2004 May 21; 279(21):22020-9; Epub 2004 Mar. 10 and Han et al., J Biol Chem. 2005 Apr. 22; 280(16):16383-92. Epub 2005 Feb. 15). After caspase-dependent cleavage and loss of the PEST domain, the anti-apoptotic function of Mcl-1L is compromised as the protein is degraded. Importantly, the resulting truncated C-terminal fragment of Mcl-1L has been shown to have pro-apoptotic functions (Herrant et al., Oncogene. 2004 Oct. 14; 23(47):7863-73; Michels et al., Oncogene. 2004 Jun. 17; 23(28):4818-27; Weng et al., J Biol Chem. 2005 Mar. 18; 280(11):10491-500. Epub 2005 Jan. 6).

Mcl-1 transcript and protein expression have been investigated in preeclamptic and normal placentae. As well the effect of varying oxygenation on Mcl-1 expression and stability has been studied.

### Methods

Collection: Placental tissues were collected from pregnancies after patient consent from 1st trimester, preeclamptic (PE), normal age-matched (AMC), term controls (C/S and normal labour), intrauterine growth restricted (IUGR), PE associated with IUGR and essential hypertension pregnancies (EH).
Explant Culture: 1st trimester explants were maintained at 37° C. in standard oxygenation (5% CO₂ in 95% air), in an atmosphere of 3% O₂/92% N₂/5% CO₂ or under hypoxia/re-oxygenation (H/R) conditions (from standard oxygenation into 2% O₂ for 3 hours and in 20% O₂ for 3 hours).
Pharmacologic Treatments Explants exposed to conditions of H/R were also incubated with 100 mM concentration of pan-caspase inhibitor z-VAD-fmk dissolved in DMSO or caspase-3-specific inhibitor z-DEVD-fink (in DMSO). Control conditions were incubated with an equivalent volume of DMSO in absence of inhibitor peptides.
Western Blotting: Fifty μg of total protein lysates were subjected to 12% SDS-PAGE followed by blotting on PVDF membranes. Membranes were incubated with a rabbit polyclonal anti-Mcl-1 antibody (Santa Cruz) at a 1:1000 dilution. Protein was detected with chemiluminescent reagent (ECL).
Quantitative PCR: RNA was isolated from various tissues using an RNeasy kit and reverse transcribed using a random hexamer approach. Mcl-1 isoform-specific primers along with a SYBR green detection system was used and analysis was performed on the DNA Engine Opticon®2 System (MJ Research). Data were normalized against 18S expression using the 2-DDCT approach.
RT-PCR-Southern Blotting: Total RNA was extracted, reverse transcribed and subjected to 20 cycles of PCR with Mcl-1L-specific primers designed outside the open reading frame. Amplified products were subjected to gel electrophoresis and blotted. Blots were hybridized with a ³²P-labeled Mcl-1 probe.
Statistics: Data was analyzed by Student's t test, significance defined as *P<0.05

### Results:

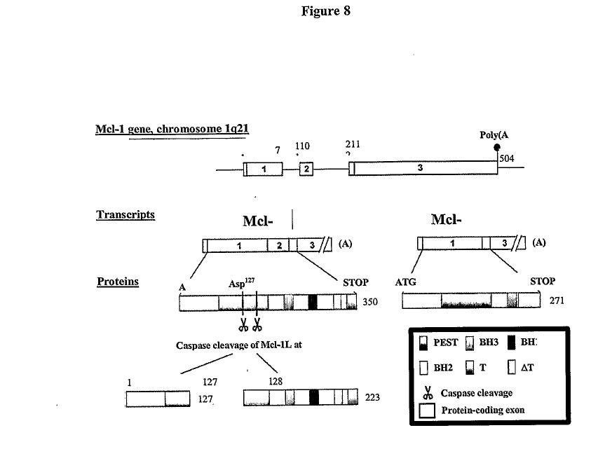

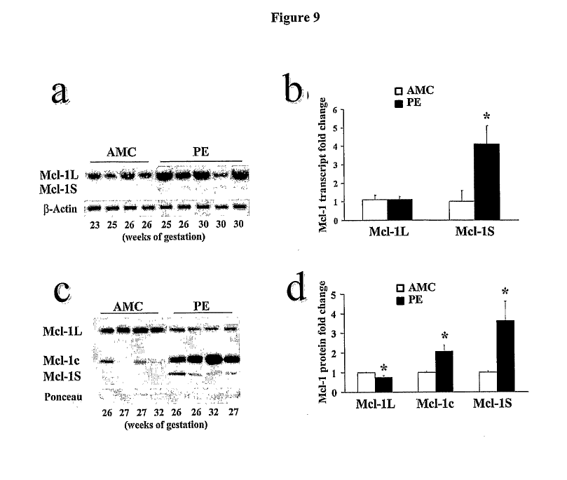

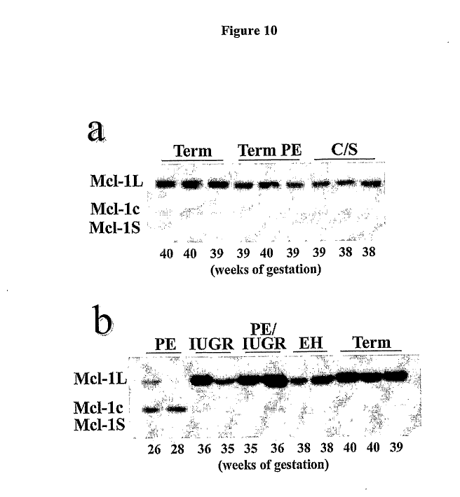

The results are shown in FIGS. 8, 9, 10, and II and are summarized below:
In early onset PE relative to AMC, the expression of anti-apoptotic Mcl-1L and pro-apoptotic Mcl-1c and Mcl-1S is respectively decreased and increased.
Mcl-1 expression does not change in late PE relative to controls or in placentae of other pregnancy-related pathologies.
Conditions of intermittent placental perfusion in vitro result in respectively increased and decreased expression of killer Mcl-1S and protector Mcl-1L molecules.
Intermittent placental oxygenation also leads to caspase-3-mediated cleavage of Mcl-1L into a pro-apoptotic fragment known as Mcl-1c (p28).

### Conclusion:

In severe preeclamptic placental tissues, aberrant placental oxygenation leads to excessive caspase activation, cleavage of pro-survival Mcl-1 isoform and switch in Mcl-1 splicing, thus tilting the trophoblast apoptotic rheostat towards a death pathway

### Example 4

### Summary

The expression of two Bcl-2 family members and interacting partners (Mtd/Bok and Mcl-1) have been examined in high altitude (HA, >3000 m), moderate altitude (MA, 1700 m) and sea-level (SL) placentae. Quantitative RT-PCR analyses demonstrated that Mcl-1L (anti-apoptotic) and Mcl-1S (pro-apoptotic) expressions are respectively increased and decreased in HA placentae relative to control tissues (MA and SL). Similarly, Mcl-1L protein levels were increased in HA vs lower altitudes. Protein expression of both pro-apoptotic Mtd-L and Mtd-P molecules were unchanged irrespective of altitude, although decreased expression of Mtd-P transcript was observed in HA relative to lower altitudes. Mtd and Mcl-1 protein expression changes were also confirmed via immunohistochemistry demonstrating increased Mcl-1 trophoblast staining in HA and unchanged Mtd expression irrespective of altitude. Immunoblotting of cleaved caspase-3 (marker of apoptosis) demonstrated markedly decreased expression of this molecule in HA placentae relative to lower altitudes. In vitro villous explants kept at 3% vs 20% showed respectively increased and decreased expression of Mcl-1L and Mcl-1S in 3%-O₂ vs standard oxygenation. Interestingly, syncytin expression (marker of trophoblast cell fusion) was decreased in HA relative to MA and SL. Thus, decreased syncytin expression and trophoblast apoptosis/turnover in HA provides a molecular adaptation to a state of chronic in vivo placental hypoxia.

A very powerful in-vivo model of chronic placental-hypoxia is pregnancies that occur at high altitude (HA, above 3000 m). At elevated altitude (near or above 3100 m), the partial atmospheric oxygen pressure is significantly diminished (pO₂≅105 mmHg/0.137 atm/2.02 psi) by approximately 34% when compared to sea level (pO₂≅158 mmgH/0.21 bar/3.05 psi) and as such a broad range of oxygen-mediated adaptive changes are required to maintain normal physiology and favorable pregnancy outcome. Placentae obtained from HA pregnancies have distinct features that differentiate them relative to moderate altitude (MA, 1700 m) or sea level pregnancies (SL) (Zamudio, High Alt Med Biol. 2003 Summer; 4(2):171-91). Placentae from high altitude pregnancies obtained from non-indigenous women have several interesting morphologic and molecular features. One important feature of these placentae is their exposure to reduced uteroplacental oxygenation caused by altitude-induced reduction in maternal arterial oxygen pressure (Zamudio, 2003). Moreover, it has been reported that critical adaptive changes that are observed during normal placental development at sea level, namely the proper trophoblast-mediated invasion and remodeling of maternal spiral arteries needed for unhindered utero-placental perfusion are compromised at high altitude (Tissot van Patot, Placenta. 2003 April; 24(4):326-35). Due to these changes, the high altitude placenta develops in a state of chronic reduced oxygenation as a result of hypobaric hypoxia experienced by the mother as well as due to sub-optimal placental development.

In response to compromised placental oxygenation at high altitude, physiologic changes aimed at ameliorating oxygen delivery to the fetus are known to occur. Some of these include increased levels of vascularization in floating placental villi, increased villous capillary density as well as thinning of villous membranes (Zamudio, 2003). As well, a proliferative villous cytotrophoblast phenotype has also been described in HA placentae perhaps due to placental hypoxia, a known inducer of trophoblast proliferation. Interestingly, high altitude placentae also exhibit reduced perisyncytial fibrin-type fibrinoid deposition when compared to placentae from lower altitude pregnancies. The significance of reduced fibrin deposition is unknown. It is yet unclear whether this change is related to an altered trophoblast apoptotic rheostat affecting trophoblast turnover.

Although high altitude placentae exhibit many beneficial adaptive changes, the rate of favorable pregnancy outcome in this environment is decreased. Notably, HA pregnancies are associated with a higher incidence of maternal and fetal complications. The rate of preeclampsia, intrauterine growth restriction (IUGR) and preterm labor are significantly increased in high altitude pregnancies (more than 2-4 fold) (Tissot van Patot, 2003). It is important to highlight that pregnancies complicated by preeclampsia as well as IUGR exhibit similar histopathologic features to that of HA pregnancies where maternal spiral arteries remodeling is incomplete (Tissot van Patot, 2003). This failure in maternal vessel remodeling may be responsible for placental hypoxia and oxidative stress.

The expression of Mcl-1 in pregnancies complicated by preeclampsia, and how in vitro conditions and a unique altitude-induced in vivo model of chronic placental hypoxia affect the expression of these molecules were investigated. Additionally, the expression of marker syncytin and cleaved caspase-3, markers of trophoblast cell differentiation/fusion and cell death respectively, were also examined in high altitude relative moderate altitude and sea level pregnancies.

### Materials and Methods

Tissue Sampling. Collection was in accordance with participating institutions' ethics guidelines. Severe early-onset preeclampsia was diagnosed based on the American College of Obstetrics and Gynecology (ACOG) criteria. Preeclamptic placentae (PE, n=20) and preterm normotensive age-matched control placentae (AMC, n=20) were collected from deliveries at Mount Sinai Hospital. Areas with calcified, necrotic or visually ischemic tissue were omitted from sampling. All preterm and term control groups did not show clinical or pathological signs of preeclampsia, infections or other maternal or placental disease. First-trimester human placental tissues (6-12 weeks of gestation, n=10) were obtained from elective terminations of pregnancies by dilatation and curettage in Toronto. High altitude (HA) and moderate altitude (MA, used as control) placental samples (n=16 each) were collected from pregnancies in Leadville (3100 m) and Denver (1700 m), Colorado, USA. HA and MA placentae were obtained from healthy normal vaginal deliveries from term normotensive patients. 15 normotensive placentae obtained from term deliveries at sea-level (SL, Toronto) were also included as an additional control. Due to organ heterogeneity, multiple specimens were sampled from central and peripheral regions and from both the maternal and fetal sides of term and age-matched control placentae. As the level of perfusion is different depending on location within the placenta, multiple specimens were sampled from central and peripheral regions on both maternal and fetal sites. Areas with calcified, necrotic or visually ischemic tissue were omitted from sampling. Subjects suffering from diabetes, essential hypertension, kidney disease or infections were excluded. The AMC, SL, MA and HA groups did not show clinical or pathological signs of preeclampsia, infection or other placental disease.
Human Chorionic First Trimester Villous Explant Culture and zVAD-fmk Treatments. Explant cultures were performed as previously described (Caniggia, 2000). Briefly, placental tissues were placed in ice-cold PBS and processed within 2 hours of collection. Tissues were aseptically dissected to remove decidual tissue and fetal membranes. Small fragments of placental villi (15-20 mg wet weight) were teased apart, placed on Millicell-CM culture dish inserts (Millipore Corp., Bedford, Mass., USA) pre-coated with 0.2 mL of undiluted Matrigel (Collaborative Biomedical Products, Bedford, Mass., USA), and put in a 24-well culture dish. Explants were cultured in serum-free DMEM/F12 (GIBCO BRL, Grand Island, N.Y., USA) supplemented with 100 μg/mL streptomycin, 100 U/mL penicillin, and incubated overnight at 37° C. in 5% CO₂ in air to allow attachment. Explants were maintained in standard condition (5% CO₂ in 95% air) or in an atmosphere of 3% O₂/92% N₂/5% CO₂ for 48 hrs at 37° C. Explants from more than 10 different placentae in more than 11 separate experiments were used. A minimum of 3 explants per experimental condition (per 3% O₂ vs. 20% O₂) was used at all times. Explants were exposed to hypoxia-reoxygenation (H/R) as previously described (Example 1; Hung, Circ Res. 2002 Jun. 28; 90(12):1274-81) in presence of 100 μM of the pan-caspase inhibitor zVAD-fmk dissolved in DMSO (equivalent volume of DMSO alone used in control conditions).
RNA Analysis. RNA extraction was performed using a Rneasy Mini Kit (Qiagen), reverse transcribed using a random hexamer approach, and amplified by 40 cycles of quantitative PCR (15 minutes at 95° C., cycle: 30 seconds at 95° C., 30 seconds at 60° C. and 30 seconds at 72° C.). Quantitative PCR was performed using the SYBR Green I dye DyNamo™ HS kit (MJ Research) based on the manufacturer's protocol using isoform specific primers for Mtd-L and Mtd-P (Mtd-L: Forward 5′-GCCTGGCTGAGGTGTGC-3′, Mtd-P: Forward 5′-GCGGGAGAGGCGATGA-3′, Reverse (both L and P) 5′-TGCAGAGAAGATGTGGCCA-3′). (Mcl-1L: Forward 5′-ATGCTTCGGAAACTGGACAT-3′, Mcl-1S: Forward 5′-CCTTCCAAGGATGGGTTTG-3′, Mcl-1 reverse (both L and S) 5′-CTAGGTTGCTAGGGTGCAA-3′). For syncytin and cytokeratin 7 analyses, qRT-PCR was performed using Assays-on-Demand™ Taqman primers and probe (Applied Biosystems, Foster City, Calif.). Analysis was done using the DNA Engine Opticon®2 System (MJ Research). Data for all qPCR analyses were normalized against expression of 18S ribosomal RNA as previously described (Livak, 2001).
Western Blot Analysis. Western blot analyses were performed as previously described (MacPhee, 2001). Briefly, 50 μg of total protein form placental tissue or cell line was subjected to 12% (wt/vol) SDS-PAGE. Membranes were probed at 4° C. overnight with a 1:1000 dilutions of a rabbit polyclonal Mtd antibody capable of recognizing all isoforms as described in Example 1, Mcl-1-specific rabbit polyclonal antibody (SC-819 clone S-19 from Santa Cruz Biotechnology, Santa Cruz, Calif.), specific cleaved caspase-3 (Asp 175) (5A1) or specific cleaved caspase-8 (Asp374) rabbit polyclonal antibodies (Cell Signaling, Beverly, Mass.). For Mtd and Mcl-1 antibodies, pre-immune serum and competing peptides were used as controls. After overnight incubation, membranes were washed with TBS/T and incubated for 60 minutes at room temperature with 1:5000 diluted horseradish peroxidase-conjugated anti-rabbit (Santa Cruz Biotechnology). Blots were exposed to chemiluminescent ECL-plus reagent (Amersham, Piscataway, N.J.). All blots were confirmed for equal protein loading using ponceau staining.
Immunohistochemistry. Immunohistochemical analyses were performed using an avidin-biotin-based immunoperoxidase approach, as previously described (Caniggia, 1999). In brief, nonspecific binding sites were blocked using 5% (vol/vol) normal goat serum (NGS) and 1% (wt/vol) BSA in Tris-buffer. Slides were incubated overnight at 4° C. with a 1:200 dilution of rabbit polyclonal anti-Mtd or anti-Mcl-1 antibodies. After washing, slides were probed with 300-fold dilution of biotinylated goat anti-rabbit or goat anti-mouse IgG (Vector Laboratories, Burlingame, Calif.) for 1 hour at 4° C. Avidin-biotin complex was applied for 1 hour. Slides were developed in 0.075% (wt/vol) 3,3-diaminobenzidine in PBS (pH 7.6) containing 0.002% (vol/vol) H₂O₂, giving rise to a brownish product. Slides were counterstained with hematoxylin, dehydrated in an ascending ethanol series, cleared in xylene, and mounted. In control experiments, primary antibodies were replaced with blocking solution (5% [vol/vol] NGS and 1% [wt/vol] BSA).

### Results

### Mcl-1L and Mcl-1S Transcript and Protein Expression in Preeclampsia

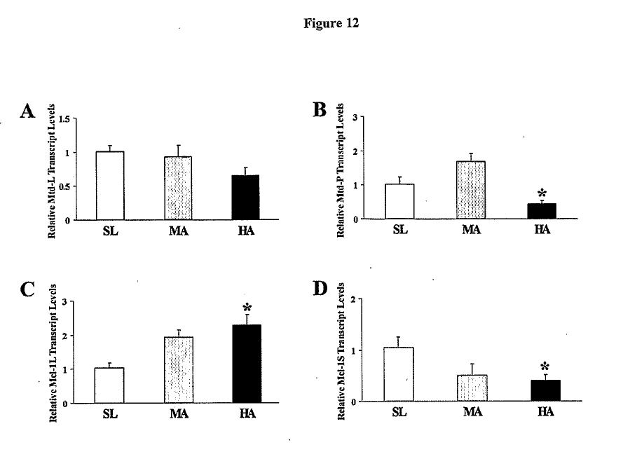

The transcript and protein expression of the 2 isoforms of Mcl-1 (the pro-apoptotic Mcl-1S and that of the anti-apoptotic Mcl-1L, the binding partner of Mtd-L) were examined in placental tissues from early severe-onset preeclamptic patients (PE) relative to age-matched control patients (AMC). RT-PCR followed by hybridization with a ³²P-labeled Mcl-1 specific probe revealed expression of both Mcl-1 transcripts in PE and AMC placentae (FIG. 12A). While the expression of Mcl-1L was variable between PE and AMC, the transcript expression of Mcl-1S was observed to increase in PE placentae relative to controls. Transcript expression of Mcl-1 isoforms was quantified using isoform-specific primers in qRT-PCR. While the expression of Mcl-1L was unchanged between PE and AMC, the expression of Mcl-1S was observed to significantly increase in PE placentae (4 fold, p=0.001) relative to control tissues, validating the earlier observations.

The Mcl-1 protein expression was examined. Western blot analyses showed a marked switch in the expression of the Mcl-1 isoforms between PE and AMC. In AMC tissues, prominent expression of the Mcl-1L isoform (around 37 kDa) was observed which markedly declined in expression in PE samples (representative blot, FIG. 12C). Interestingly, the expression of 2 shorter Mcl-1-specific bands (between the 30 to 25 kDa markers) was observed, increasing in expression in preeclamptic relative to AMC tissues. These two protein bands migrated at the relative molecular weights believed to correspond to p28 (a pro-apoptotic caspase-3-mediated cleaved byproduct of Mcl-1L (aa 128-350)) and Mcl-1S (a pro-apoptotic splice isoform with apparent molecule weight of approximately 27-28 kDa).

### Mcl-1 Transcript and Protein Expression in Villous Explants Under Conditions of Varying Oxygenation.

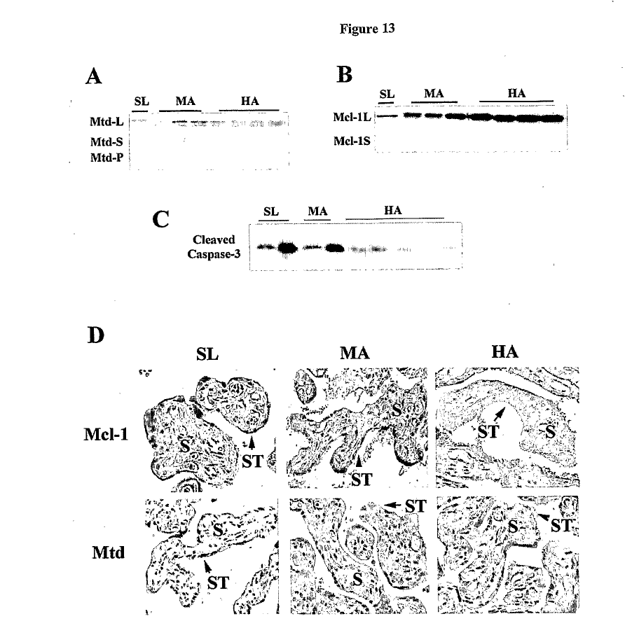

In order to confirm the observations of differential expression of Mcl-1 isoforms between PE and AMC, functional studies were performed in vitro using first trimester explants exposed to conditions of hypoxia-reoxygenation (H/R). Intermittent oxygenation was chosen as it was previously established to be an important inducer of caspase activation and trophoblast apoptosis in vitro (Hung, 2002). To determine whether the expression of the shorter Mcl-1 bands was affected by caspase activity, explants were exposed to H/R in presence or absence of zVAD-fmk, a broad-based inhibitor of caspase activity, and previously validated to be an inhibitor of caspase-mediated Mcl-1L cleavage. Explants exposed to H/R (in presence of DMSO) demonstrated a notable switch in banding pattern of Mcl-1 protein where Mcl-1L declined in expression with a concomitant increased expression of Mcl-1S and to a greater extent the formation of p28, relative to untreated control tissue (representative immunoblot, FIG. 13A). Interestingly, exposure of H/R-treated explants to 100 μM concentration of zVAD-fnk prevented the cleavage of Mcl-1L into its caspase-cleaved fragment p28 and lead to even higher expression of Mcl-1S relative to control H/R and particularly when compared to untreated control conditions (FIG. 13A). As such, inhibition of caspase activity under conditions of intermittent oxygenation results in increased expression of Mcl-1L and Mcl-1S as the PEST sequence these proteins will be cleaved by activated caspases. To quantify the switch of Mcl-1 protein expression in H/R conditions (presence and absence of zVAD-fmk) relative to untreated control tissues densitometric analysis was performed.

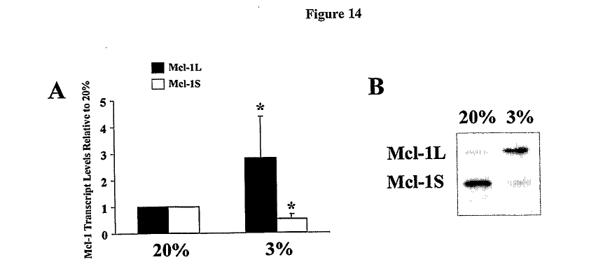

As changes were observed with respect to Mcl-1 expression in the altitude-induced model of chronic placental hypoxia relative to moderate and sea-level samples, whether Mcl-1 expression also changed under varying oxygenation conditions in vitro was also examined. Mcl-1 transcript and protein levels were analyzed in first trimester villous explants exposed to 20% O₂ (standard oxygenation) and 3% O₂ (reduced oxygenation). FIG. 14A depicts Mcl-1 transcript expression under 3% and 20% oxygen. Mcl-1L and Mcl-1S increased and decreased respectively both at the messenger and protein levels under 3% oxygenation when compared to 20% (FIGS. 14C and D). The data with respect to Mcl-1 gene and protein expression further substantiated the observations that were made with respect to the transcript and protein expression patterns of this molecule in our altitude-induced model of placental hypoxia relative to lower altitude scenarios.

### Pro-Apoptotic and Anti-Apoptotic Mtd and Mcl-1 Transcripts are Respectively Decreased and Increased in Chronic Placental Hypoxia

As the expression of Mtd isoforms L and P was previously shown to increase in preeclamptic placentae as well as under conditions of oxidative stress, their expression was examined in placentae from HA, MA and SL pregnancies. FIGS. 12A and 12B illustrate the transcript expression of Mtd-L and Mtd-P respectively in HA, MA and SL tissues as assessed by quantitative real-time PCR. The expression of Mtd-L was not statistically different between SL, MA and HA placental samples, although a slight decrease was observed in HA samples when compared to MA and SL conditions. Interestingly, Mtd-P, previously shown to be significantly increased in early onset severe preeclampsia when compared to normotentive age-matched control tissues, was observed to significantly decrease in HA samples when compared to MA and SL tissues (FIG. 12B).

In contrast to Mtd, Mcl-1 isoforms exhibited differential messenger RNA expression between HA, MA and SL placental samples. FIGS. 12C and D respectively depict the relative transcript expressions of Mcl-1L and Mcl-1S in HA, MA and SL tissues. While the anti-apoptotic Mcl-1L transcript was shown to significantly increase (approximately 2-fold) in HA and MA when compared SL, the transcript expression of the pro-apoptotic isoform Mcl-1S was shown to significantly decrease (approximately by 50%) in HA and MA relative to SL samples (FIGS. 12C and D). These data collectively demonstrate a shift of transcript expression towards protective/anti-apoptotic isoforms within the Mtd-Mcl-1 rheostat in HA vs MA and SL.

### Protein Localization of Mtd and Mcl-1 in High Altitude Placentae

FIG. 13A depicts a representative immunoblot of Mtd in placental samples from SL, MA and HA pregnancies. Although the expression of all known Mtd isoforms (L=28 kDa, S=18 kDa and P=15 kDa) is observed there was no apparent differences in the expression of any the isoforms between SL, MA and HA samples (FIG. 13A). It is also important to note that Mtd-P expression is almost undetectable in these tissues, in contrast to what was previously reported in early-onset severe preeclamptic pregnancies (See Example 1). As well, Mtd protein expression correlated with messenger RNA expression. Moreover, similar to Mcl-1 transcript expression, the protein expression in SL, MA and HA samples shows a notable increase in Mcl-1L in HA and to a lesser extent in MA when compared to sea level samples (FIG. 13B). The Mcl-1S molecule had very weak expression at the protein level in SL, MA and HA samples with no apparent changes in expression between these conditions (FIG. 13B).

Mcl-1 immunoreactivity in placental sections from SL, MA and HA samples was predominantly observed in trophoblast cell layers (FIG. 13D, top panels). The expression of Mcl-1 was observed to be markedly increased in HA sections when compared to MA and SL samples. This finding further corroborates what was observed when performing Western blot analyses to assess Mcl-1 protein expression. Stromal regions were observed to be Mcl-1 negative. As well, similar to Mcl-1 immunoreactivity, Mtd immunolocalization is also predominantly expressed in trophoblast cell layers. Similar to Mtd immunoblotting analyses, notable expression changes were not observed with respect to Mtd protein between SL, MA and HA sections (FIG. 13D, bottom panels). It is also histo-morphologically interesting to observe that HA samples and to a lesser extent MA tissues exhibit an increased villous capillary density when compared to SL section, which represents a unique adaptive response to altitude-induced reduced oxygenation.

### Mtd, Mcl-1 and Cleaved Caspase-3 Protein Expression and Expression of Syncytin In MA, HA and SL Placentae

Interestingly, a representative immunoblot of cleaved caspase-3 (previously demonstrated to be a reliable marker of trophoblast apoptosis) (Hung, 2002) performed on protein lysates from HA, MA and SL placental tissues showed reduced cleavage of this molecule (an executioner of apoptosis) in HA samples relative to lower altitudes (FIG. 13C). This finding corroborates the data demonstrating a shift of the Mtd-Mcl-1 rheostat towards protective/anti-apoptotic state in HA and thereby indicates a decreased level of apoptotic-mediated cell death in placentae subjected to chronic placental hypoxia in vivo.

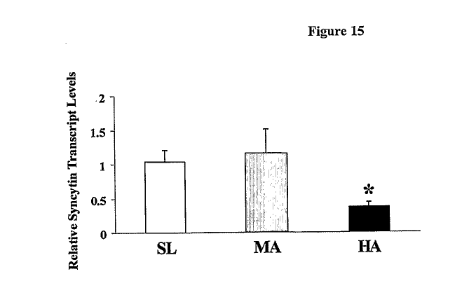

Studies have thus far postulated that trophoblast apoptosis is a normal driving force in mediating cytotrophoblast to syncytiotrophoblast cell differentiation and ultimately the turnover and shedding of a dying syncytium in the maternal uteroplacental circulation (Huppertz, B. et al, Histochem Cell Biol. 1998 November; 110(5):495-508; Huppertz, B. et al. Lab Invest. 1999 December; 79(12):1687-702.). As placentae from high altitude pregnancies appear to exhibit a thinning of trophoblast membranes possibly due to reduced trophoblast cell death/turnover, the expression of a trophoblast differentiation marker known syncytin was investigated in these samples. Recent studies have demonstrated that the syncytin molecule, a captive retroviral envelope protein belonging to a recently identified human endogenous defective retrovirus, known as HERV-W, is a highly expressed molecule in human trophoblast cells and has been shown be play a key role in cytotrophoblast cell fusion (Frendo, Mol Cell Biol. 2003 May; 23(10):3566-74). Additionally, previous studies have demonstrated that the expression of syncytin is a reliable marker for assessing trophoblast cell fusion/differentiation. Using quantitative real-time PCR the relative transcript expression of this molecule was measured in placental tissues from SL, MA and HA pregnancies. Relative to MA and SL tissues, HA samples have a significantly reduced level of syncytin expression, which may present a possible explanation of a reduced rate of trophoblast turnover leading to thinning of the villous membrane observed in these pregnancies (FIG. 15).

### Discussion

This study has demonstrated that chronic reduced placental oxygenation provides a protective and adaptive environment against trophoblast cell death and turnover. The in vivo and in vitro oxygen models demonstrated that reduced placental oxygenation shifts the Mtd-Mcl-1 apoptotic rheostat towards a protective state against apoptotic-mediated cell death. Respectively, increased and decreased expressions of the anti-apoptotic Mcl-1L and Mcl-1S molecules under reduced O₂ relative to standard oxygenation conditions is the first evidence that Mcl-1 gene expression may be partly regulated by oxygen in placental tissues. As well, this is the first account of the differential splicing of the Mcl-1 transcript in varying oxygenation conditions. Interestingly, Mtd-L expression, the interacting partner of Mcl-1L, as well as the expression of Mtd-P, appeared to be unaffected by a state of chronic decreased oxygenation. Mtd-P transcript expression, previously shown to be increased in conditions of oxidative stress as well as in placentae from severe early onset preeclamptic patients (see Example 1), was decreased in placental tissues obtained from high altitude pregnancies relative to moderate altitude and sea level tissues. These findings along with the observed decreased expression of cleaved caspase-3 in HA placentae relative to lower altitudes provides the first molecular evidence of decreased apoptotic-mediated trophoblast cell death under conditions of chronic placental hypoxia. This event may hence indirectly affect the state of villous trophoblast cellular differentiation and turnover in high altitude. In fact it has been observed that formation of apoptotic-syncytial knots in high-altitude placentae (above 3600 m) is significantly decreased relative to lower altitudes (Mayhew, 2002). This finding supports the findings herein which are suggestive of reduced apoptotic-mediated trophoblast turnover.

The data herein indicates that with respect to Mcl-1 expression, there is a shift towards reduced apoptosis in low oxygen as decreased pro-apoptotic Mcl-1S and increased anti-apoptotic Mcl-1L expression were observed under reduced oxygenation relative to standard conditions. This is the first evidence of a possible oxygen-mediated role with respect to regulation of Mcl-1 expression.

The data with respect to syncytin expression (significantly decreased in HA relative to MA and SL samples) in conjunction with a decreased rate of trophoblast cell death in high-altitude placental samples may provide a molecular explanation for the observed decrease in the thickness of the placental syncytium in high-altitude placentae. The decreased expression of the syncytin molecule, previously shown to be a key regulator of trophoblast cell fusion, may possibly decelerate the fusion rate of cytotrophoblast cells in high altitude placentae and as such may limit the rate of de-novo syncytium synthesis and maintenance at the expense of normal shedding/deportation (turnover) of dying syncytial debrits (syncytiotrophoblast microfragments) in the maternal circulation. This imbalance in the dynamic rate of syncytial renewal and shedding may thus provide a molecular explanation with respect to the thinned syncytial phenotype observed in high altitude placentae. Studies have shown that in conditions of reduced oxygenation, syncytin as well as its binding receptor (ASCT2) are downregulated relative to standard oxygenation conditions. As well, studies have also reported decreased expression of syncytin in pregnancies complicated by preeclampsia (Knerr, Am J Obstet Gynecol. 2002 February; 186(2):210-3; Lee, Placenta. 2001 November; 22(10):808-12). These findings support the observations of reduced syncytin expression in conditions of altitude-induced in vivo placental hypoxia relative to normoxic moderate-altitude and seal-level scenarios.

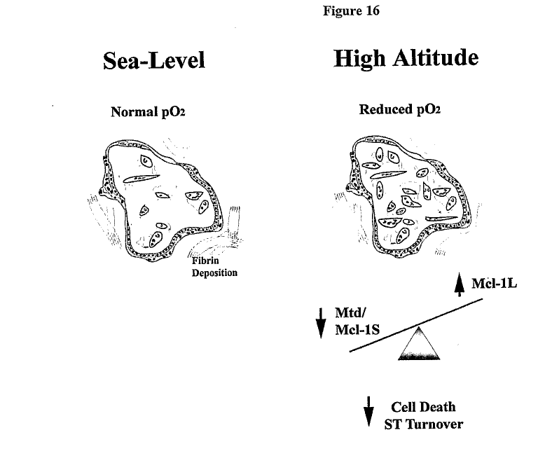

In conclusion, this study provides the first evidence of the Mtd-Mcl-1 rheostat in regulating trophoblast apoptosis under conditions of in vivo and in vitro placental hypoxia (FIG. 16). As well, syncytial thinning in high altitude placentae may be due to a downregulation of syncytin expression due to altitude-induced placental hypoxia. As such in high altitude placentation, decreased trophoblast cell death in conjunction with decreased trophoblast turnover/differentiation may present an important adaptive response to chronic placental hypoxia and as such improve the outcome of pregnancies at high altitude

### Example 5

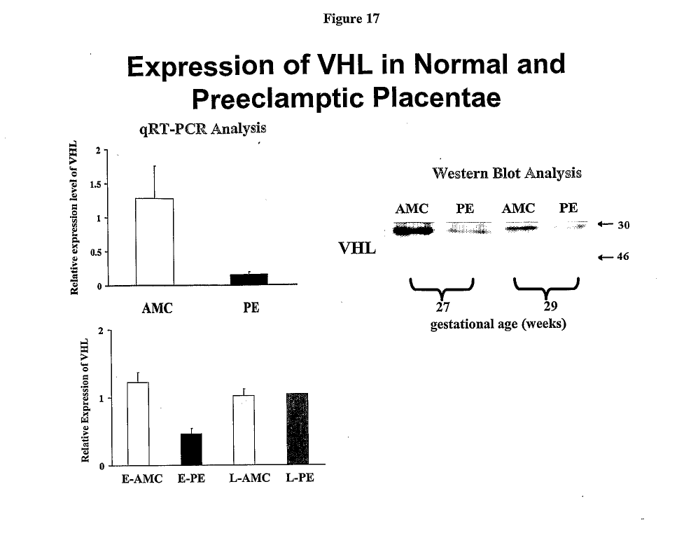

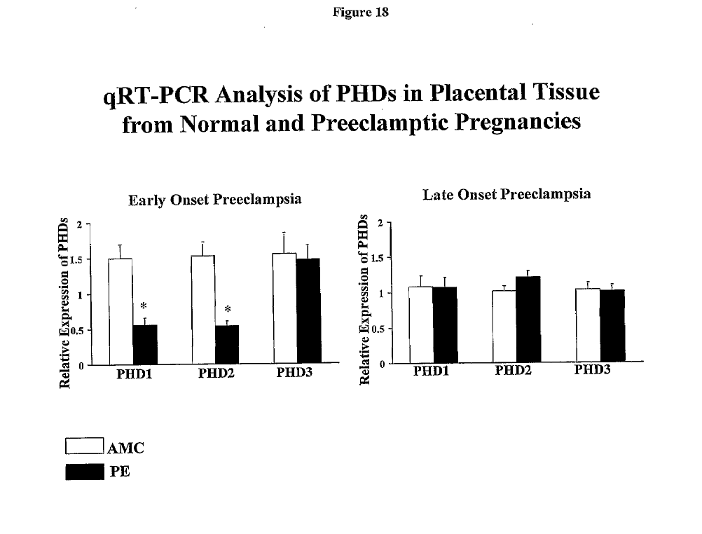

Expression of VHL and PHD1, PHD2, and PHD3 have been investigated in preeclamptic and normal placentae. FIG. 17 are graphs and an immunoblot showing the expression of VHL is decreased in preeclamptic placentae, in particular in early onset preeclamptic placentae, relative to age-matched controls. FIG. 18 are graphs showing the results of qRT-PCR analysis showing that the expression of PHD1 and PHD2 is decreased in placental tissue from early onset preeclamptic pregnancies relative to age-matched controls.

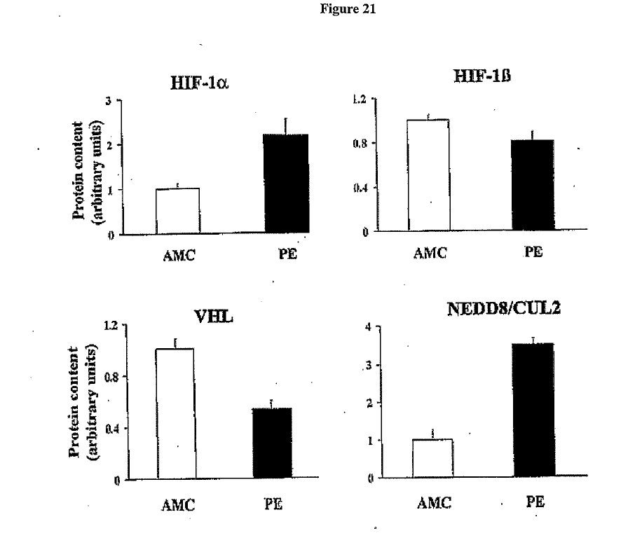

Expression of NEDD8/CUL2 has also been investigated in in preeclamptic and normal placentae and it was found that the levels of NEDD8/CUL2 are decreased in preeclamptic placentae (see FIG. 21).

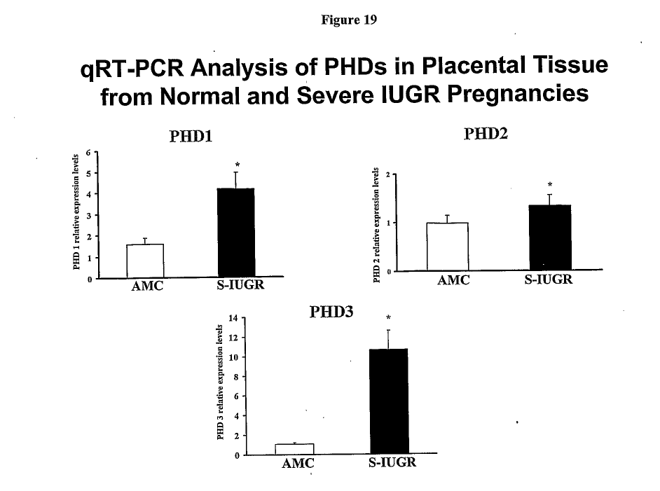

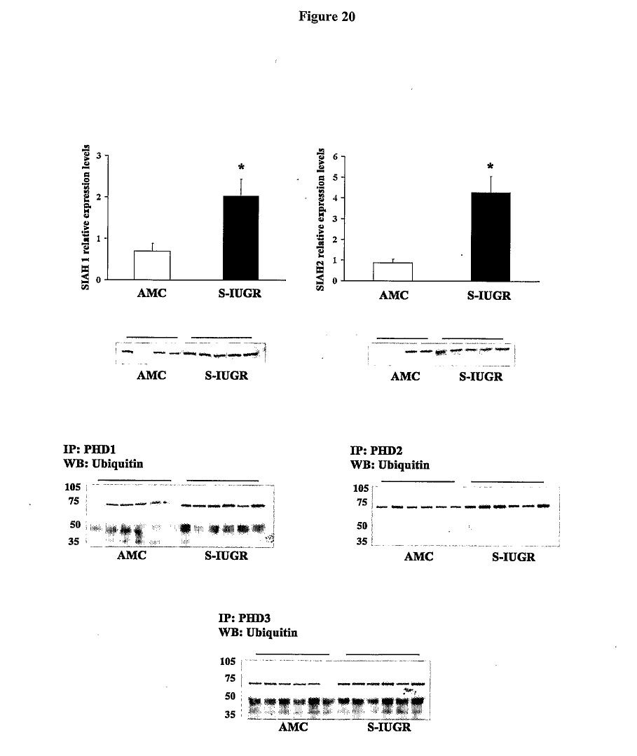

Expression of PHDs and SIAH1/2 have been investigated in placental tissue from normal and severe IUGR. FIG. 19 are graphs shows PHD1, PHD2, and PHD3 are elevated in placental tissue from severe IUGR pregnancies relative to age-matched controls. FIG. 20 are graphs and immunoblots showing that SIAH1 and SIAH2 are increased in placental tissue from severe IUGR pregnancies relative to age-matched controls and PHDs are elevated in tissue from severe IUGR pregnancies.

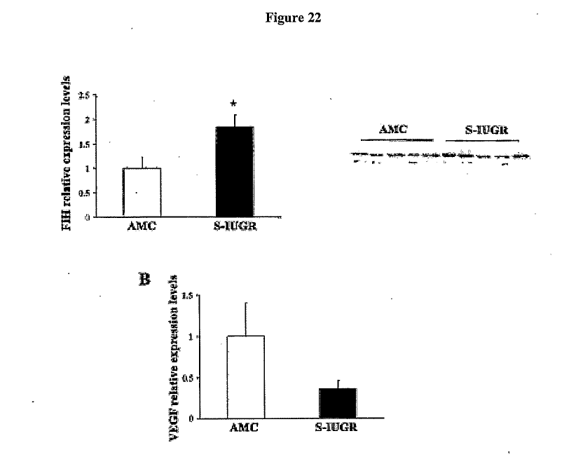

Expression of FIH was also investigated in placental tissue from normal and severe IUGR. The expression of FIH was increased in placental tissue from severe IUGR pregnancies relative to age-matched controls. In addition, expression of VEGF was investigated in placental tissue from normal and severe IUGR. The expression of VEGF was decreased in placental tissue from severe IUGR pregnancies relative to age-matched controls (see FIG. 22).

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Clinical Parameter of Preeclamptic and Control Participants

| | Preeclamptic Subjects | Control Subjects |
|---|---|---|
| | n = 66 | n = 59 |
| Mean Maternal Age | 29.9 ± 6.2 | 31.8 ± 5.6 |
| (Years) | | |
| Mean Gestational Age | Preterm: 30.8 ± 3.1 (25-34, n = 51) | Preterm: 30.4 ± 3.4 (23-36, n = 36) |
| (Range in weeks) | Term: 38.9 ± 1.1 (37-41, n = 15) | Term: 39.2 ± 1.0 (37-41, n = 23) |
| Blood Pressure | Systolic: 177 ± 7.2 | Systolic: 112 ± 6.6 |
| | Diastolic: 114 ± 4.5 | Diastolic: 68 ± 6.0 |
| Proteinuria | 3.0 ± 1 | Absent |
| Edema | Present: 81% | Absent |
| | Absent: 19% | |
| Fetal Weight (g) | Preterm A.G.A.: 1476 ± 456 (n = 40) | Preterm A.G.A.: 1497 ± 628 |
| | Preterm IUGR: 1065 ± 506 (n = 11) | Term A.G.A.: 3359 ± 409 |
| | Term A.G.A.: 3295 ± 478 (n = 15) | |
| Mode of Delivery | CS: 90% | CS: 45% |
| | VD: 10% | VD: 55% |

NOTE:
Data are represented as mean ± standard deviation
Maternal age of participants ranged from 16 to 44 years
A.G.A.: Appropriate for gestational age
IUGR: Intrauterine growth restriction (<5^{th} %)
VD: Vaginal delivery
CS: Caesarian section delivery

### REFERENCES

1. Cunningham F G and Lindheimer M D (1992) Hypertension in pregnancy. N. Engl. J. Med. 326: 927-932
2. Meekins J W, Pijnenborg R, Hanssens M, McFadyen I R and van Asshe A (1994) A study of placental bed spiral arteries and trophoblast invasion in normal and severe pre-eclamptic pregnancies. Br. J. Obstet. Gynaecol. 101: 669-674
3. Aquilina J and Harrington K (1996) Pregnancy hypertension and uterine artery Doppler ultrasound. Curr. Opin. Obstet. Gynecol. 8: 435-440
4. Redman C W and Sargent I L (2000) Placental debris, oxidative stress and pre-eclampsia. Placenta 21: 597-602
5. Roberts J M and Hubel C A (1999) Is oxidative stress the link in the two-stage model of preeclampsia? Lancet 354: 788-789
6. Brosens I and Renaer M (1972) On the pathogenesis of placental infarcts in pre-eclampsia. J. Obstet. Gynaecol. Br. Comrnonw. 79: 794-799
7. Sargent I L, Germain S J, Sacks G P, Kumar S and Redman C W (2003) Trophoblast deportation and the maternal inflammatory response in pre-eclampsia. J. Reprod. Immunol. 59: 153-160
8. Huppertz B, Frank H G, Kingdom J C, Reister F and Kaufmann P (1998) Villous cytotrophoblast regulation of the syncytial apoptotic cascade in the human placenta. Histochem. Cell. Biol. 110: 495-508
9. DiFederico E, Genbacev O and Fisher S J (1999) Preeclampsia is associated with
10. widespread apoptosis of placental cytotrophoblasts within the uterinewall. Am. J. Pathol. 155: 293-301 24
11. Huppertz B, Frank H G, Reister F, Kingdom J, Korr H and Kaufmann P (1999) Apoptosis cascade progresses during turnover of human trophoblast: analysis of villous cytotrophoblast and syncytial fragments in vitro. Lab. Invest. 79: 1687-1702
12. Gross A, McDonnell J M and Korsmeyer S J (1999) BCL-2 family members and the mitochondria in apoptosis. Genes Dev. 13: 1899-1911
13. Hsu S Y and Hsueh A J (2000) Tissue-specific Bcl-2 protein partners in apoptosis: An ovarian paradigm. Physiol. Rev. 80: 593-614
14. Ratts V S, Tao X J, Webster C B, Swanson P E, Smith S D, Brownbill P, Krajewski S, Reed J C, Tilly J L and Nelson D M (2000) Expression of BCL-2, BAX and BAK in the trophoblast layer of the term human placenta: a unique model of apoptosis within a syncytium. Placenta 21: 361-366
15. Allaire A D, Ballenger K A, Wells S R, McMahon M J and Lessey B A (2000) Placental apoptosis in preeclampsia. Obstet. Gynecol. 96: 271-276
16. Levy R, Smith S D, Yusuf K, Huettner P C, Kraus F T, Sadovsky Y and Nelson D M (2002) Trophoblast apoptosis from pregnancies complicated by fetal growth restriction is associated with enhanced p53 expression. Am. J. Obstet. Gynecol. 186: 1056-1061
17. IshiharaN, Matsuo H, Murakoshi H, Laoag-Fernandez J B, Samoto T and Maruo T (2002) Increased apoptosis in the syncytiotrophoblast in human term placentas complicated by either preeclampsia or intrauterine growth retardation. Am. J. Obstet. Gynecol. 186: 158-166
18. Hsu S Y, Kaipia A, McGee E, Lomeli M and Hsueh A J (1997) Bok is apro-apoptotic Bcl-2 protein with restricted expression in reproductive tissues and heterodimerizes with selective anti-apoptotic Bcl-2 family members. Proc. Natl. Acad. Sci. USA 94: 12401-12406 25
19. Inohara N, Ekhterae D, Garcia I, Carrio R, Merino J, Merry A, Chen S and Nunez G (1998) Mtd, a novel Bcl-2 family member activates apoptosis in the absence of heterodimerization with Bcl-2 and Bcl-XL. J. Biol. Chem. 273: 8705-8710
20. Hsu S Y and Hsuch A J (1998) A splicing variant of the Bcl-2 member Bok with a truncated BH3 domain induces apoptosis but does not dimerize with antiapoptotic Bcl-2 proteins in vitro. J. Biol. Chem. 273: 30139-30146
21. Hung T H, Skepper J N, Charnock-Jones D S and Burton G J (2002) Hypoxia-reoxygenation: a potent inducer of apoptotic changes in the human placenta and possible etiological factor in preeclampsia. Circ. Res. 90: 1274-1281
22. Caniggia I, Taylor C V, Ritchie J W, Lye S J and Letarte M (1997) Endoglin regulates trophoblast differentiation along the invasive pathway in human placental villous explants. Endocrinology 138: 4977-4988
23. Zhang H, Holzgreve W and De Geyter C (2000) Evolutionarily conserved Bok proteins in the Bcl-2 family. FEBS Lett. 480: 311-313
24. Brown C Y, Bowers S J, Loring G, Heberden C, Lee R M and Neiman P E (2004) Role of Mtd/Bok in normal and neoplastic B-cell development in the bursa of Fabricius. Dev. Comp. Immunol. 28: 619-634
25. Leung D N, Smith S C, To K F, Sahota D S and Baker P N (2001) Increased placental apoptosis in pregnancies complicated by preeclampsia. Am. J. Obstet. Gynecol. 184: 1249-1250
26. Crocker I P, Cooper S, Ong S C and Baker P N (2003) Differences in apoptotic susceptibility of cytotrophoblasts and syncytiotrophoblasts in normal pregnancy to those complicated with preeclampsia and intrauterine growth restriction. Am. J. Pathol. 162: 637-643 26
27. Hsu C D, Harirah H, Basherra H and Mor G (2001) Serum soluble Fas levels in preeclampsia. Obstet. Gynecol. 97: 530-532
28. Caniggia I, Mostachfi H, Winter J, Gassmann M, Lye S J, Kuliszewski M and Post M (2001) Hypoxia-inducible factor-1 mediates the biological effects of oxygen on human trophoblast differentiation through TGFbeta(3). J. Clin. Invest. 105: 577-587
29. Genbacev O, Zhou Y, Ludlow J W and Fisher S J (1997) Regulation of human placental development by oxygen tension. Science 277: 1669-1672
30. Genbacev O, Joslin R, Damsky C H, Polliotti B M and Fisher S J (1996) Hypoxia alters early gestation human cytotrophoblast differentiation/invasion in vitro and models the placental defects that occur in preeclampsia. J. Clin. Invest. 97: 540-550
31. Clevenger C V, Thicman K, Ngo W, Chang W P, Takayama S and Reed J C (1997) Role of Bag-1 in the survival and proliferation of the cytokine-dependent lymphocyte lines, Ba/F3 and Nb2. Mol. Endocrinol. 11: 608-618
32. Linette G P, Li Y, Roth K and Korsmeyer S J (1996) Cross talk between cell death and cell cycle progression: BCL-2 regulates NFAT-mediated activation. Proc. Natl. Acad. Sci. USA 93: 9545-9552
33. Vairo G, Innes K M and Adams J M (1996) Bcl-2 has a cell cycle inhibitory function separable from its enhancement of cell survival. Oncogene 13: 1511-1519
34. Chattopadhyay A, Chiang C W and Yang E (2001) BAD/BCL-[X(L)] heterodimerization leads to bypass of G0/G1 arrest. Oncogene 20: 4507-4518
35. Fridman J S, Rehemtulla A, Hofmann A, Blau H M and Maybaum J (1998) Expression of Bcl-XS alters cytokinetics and decreases clonogenic survival in K12 rat colon carcinoma cells. Oncogene 17: 2981-2991 27
36. Townsend P A, Cutress R I, Sharp A, Brimmell M and Packham G (2003) BAG-1: a multifunctional regulator of cell growth and survival. Biochim. Biophys. Acta 1603: 83-98 36.
37. Levy R, Smith S D, Chandler K, Sadovsky Y and Nelson D M (2000) Apoptosis in human cultured trophoblasts is enhanced by hypoxia and diminished by epidermal growth factor. Am. J. Physiol. Cell. Physiol. 278: C982-C988
38. Caniggia I and Winter J L (2002) Adriana and Luisa Castellucci Award lecture 2001. Hypoxia inducible factor-1: oxygen regulation of trophoblast differentiation in normal and pre-eclamptic pregnancies—a review. Placenta 23 Suppl A: S47-S57
39. Rajakumar A, Whitelock K A, Weissfeld L A, Daftary A R, Markovic N and Conrad K P (2001) Selective overexpression of the hypoxia-inducible transcription factor, HIF-2alpha, in placentas from women with preeclampsia. Biol. Reprod. 64: 499-506
40. Sowter H M, Ratcliffe P J, Watson P, Greenberg A H and Harris A L (2001) HIF-1-dependent regulation of hypoxic induction of the cell death factors BNIP3 and NIX in human tumors. Cancer Res. 61: 6669-6673
41. Bruick R K (2000) Expression of the gene encoding the proapoptotic Nip3 protein is induced by hypoxia. Proc. Natl. Acad. Sci. USA 97: 9082-9087
42. Yakovlev A G; Di Giovanni S, Wang G, Liu W, Stoica B and Faden A I (2004) BOK and NOXA are essential mediators of p53-dependent apoptosis. J. Biol. Chem. 279: 28367-28374
43. Graeber T G, Peterson J F, Tsai M, Monica K, Formace A J and Giaccia A J (1994) Hypoxia induces accumulation of p53 protein, but activation of a G1-phase checkpoint by low oxygen conditions is independent of p53 status. Mol. Cell. Biol. 14: 6264-6277 28
44. ACOG practice bulletin (2002) Diagnosis and management of preeclampsia and eclampsia. Number 33, January 2002. American College of Obstetricians and Gynecologists. Int. J. Gynaecol. Obstet. 77: 67-75
45. Livak K J and Schmittgen T D (2001) Analysis of relative gene expression data using realtime quantitative PCR and the 2(-Delta Delta C(T)) Method. Methods 25: 402-408
46. MacPhee D J, Mostachfi H, Han R, Lye S J, Post M and Caniggia I (2001) Focal adhesion kinase is a key mediator of human trophoblast development. Lab. Invest. 81: 1469-1483
47. Jurisicova A, Johnson A L, Casper R F and Tilly J L (2000) Developmentally regulated expression of the pro-apoptotic Bcl-2 family member, Mtd/Bok, in oocytes and preimplantation embryos. The Endocrine Society 131: 316
48. Herrmann M, Lorenz H M, Voll R, Grunke M, Woith W and Kalden J R (1994) A rapid and simple method for the isolation of apoptotic DNA fragments. Nucleic Acids Res. 22: 5506-5507

1. An isolated polynucleotide comprising:
(a) a nucleic acid sequence encoding a polypeptide comprising an amino acid sequence of SEQ ID NO: 1,
(b) a nucleic acid sequence of SEQ ID NO: 2;
(c) a nucleic acid sequence complementary to (a) or (b);
(d) a degenerate form of a nucleic acid sequence of (a) or (b);
(e) a nucleic acid sequence capable of hybridizing under stringent conditions to polynucleotide (a), (b), or (c);
(f) a nucleic acid sequence encoding a truncation, an analog, an allelic or species variation of a polypeptide comprising an amino acid sequence of SEQ ID NO: 1;
(g) a fragment, or allelic or species variation of polynucleotide (a), (b), or (c);
(h) a variant of polynucleotide (a) or comprising a sequence of SEQ ID NO. 4, wherein the nucleic acid sequence encodes a domain having the ability to interact with an anti-apoptotic molecule, and wherein the variant comprises an isolated nucleic acid sequence having at least one mutation resulting in loss of the ability of the domain to interact with the anti-apoptotic molecule; or
(i) a variant of polynucleotide (a) or comprising a sequence of SEQ ID NO. 4, wherein the nucleic acid sequence comprises a second exon encoding part of a domain having the ability to interact with an anti-apoptotic molecule, and wherein the variant is selected from the group consisting of:
(i) isolated nucleic acid sequences lacking the second exon,
(ii) isolated nucleic acid sequences having at least one mutation in the second exon resulting in loss of the ability of the domain to interact with the anti-apoptotic molecule, and
(iii) isolated nucleic acid sequences lacking splice sites defining the second exon.

2-6. (canceled)

5. A vector or host cell comprising a polynucleotide of claim 1.

6. (canceled)

7. An isolated polypeptide encoded by a polynucleotide of claims 1.

8. An isolated polypeptide according to claim 7 comprising an amino acid sequence of SEQ ID NO:1.

9. (canceled)

10. An antibody having specificity against an epitope of a polypeptide of claim 7.

11-12. (canceled)

13. A method of diagnosing or monitoring a condition associated with a polynucleotide of claim 1 in a subject by determining the presence of the polynucleotide in a sample from the subject.

14. (canceled)

15. A method according to claim 13, wherein the condition is selected from the group consisting of a condition requiring regulation of trophoblast cell death, differentiation, invasion, and cell fusion and turnover and preeclampsia.

16. (canceled)

17. A method of identifying a substance which associates with a polypeptide of claim 7 comprising:
reacting the polypeptide with at least one substance which potentially can associate with the polypeptide, under conditions which permit association between the substance and the polypeptide, and
removing or detecting polypeptide associated with the substance, wherein detection of associated polypeptide and substance indicates the substance associates with the polypeptide.

18. A method for evaluating a compound for its ability to modulate the biological activity of a polypeptide of claim 7 comprising:
providing the polypeptide with a substance which associates with the protein and a test agent under conditions which permit the formation of complexes between the substance and polypeptide, and
removing the complexes or detecting the complexes.

19. A method for identifying inhibitors of a Mtd-P Polypeptide interaction, comprising:
providing a reaction mixture including a polypeptide of claim 7 and a substance that binds to the polypeptide, or at least a portion of each which interact; contacting the reaction mixture with one or more test agents;
identifying compounds which inhibit the interaction of the polypeptide and substance.

20. A method for detecting a polynucleotide of claim 1 in a biological sample comprising:
hybridizing a polynucleotide according of claim 1 to nucleic acids of the biological sample, thereby forming a hybridization complex; and
detecting the hybridization complex wherein the presence of the hybridization complex correlates with the presence of a polynucleotide in the biological sample.

21. (canceled)

22. A method for detecting a condition requiring modulation of or involving trophoblast cell death, differentiation, invasion, cell fusion and turnover, or combinations thereof, or a predisposition to such condition, comprising:
producing a profile of levels of a polynucleotide of claim 1 in a sample from a subject, and
comparing the profile with a reference to identify a profile for the subject indicative of the condition.

23. A method of claim 22 further comprising preparing a profile of one or more of Mcl-1 isoforms TGFβ3, TGFβ1, HIF-1α, HIF-1β, HIF-2 α, VHL, cullin 2, NEDD8, PHD1, PHD2, PHD3, Siah1/2, syncytin, Fas, VEGF, FIH, cleaved caspase, p53, or polynucleotides encoding same.

24. A method for classifying preeclampsia comprising detecting a difference in the expression of a plurality of markers relative to a control, the plurality of markers or polynucleotide markers comprising or selected from the group consisting of Mtd-P, Mtd-L, Mtd-S, Mcl-1 isoforms, TGFβ3, TGF β1, HIF-1α, HIF-1β, HIF-2 α, VHL, cullin 2, NEDD8, Mcl-1, PHD1, PHD2, PHD3, Siah1/2, syncytin, Fas, VEGF, FIH, cleaved caspase, p53, polynucleotides encoding same, or a combination thereof.

25. (canceled)

26. A microarray for distinguishing conditions requiring modulation of trophoblast cell death, differentiation, invasion, cell fusion and turnover, or combinations thereof comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality comprising at least 2, 3, 4, 5, 6, 7, 8, 9, 10, 11, 12, 13, or 14 of the genes corresponding to the polynucleotides encoding Mtd-P, Mtd-L, Mtd-S, Mcl-1 isoforms, TGFβ3, TGFβ1, HIF-1α, HIF-1β, HIF-2α, VHL, PHD1, PHD2, PHD3, Siah1/2, syncytin, VEGF, FIH, cullin 2, NEDD8, Fas, cleaved caspase, p53, or combinations thereof.

27. A method for diagnosing early onset preeclampsia comprising comparing levels of at least two, three, four, five, six, seven, eight, nine, or ten of Mtd-P, Mtd-L, Mcl-1S, Mcl-1L truncation, TGFβ3, TGFβ1, HIF1α, PHD1, PHD2, Siah1/2, cleaved caspase, VHL, or polynucleotides encoding same in a sample from a subject to the corresponding levels in a control.

28-29. (canceled)

30. A method according to of claim 27 wherein a 2 to 10 fold increase in Mtd-P or a polynucleotide encoding same, or a 2 to 10 fold increase in Mtd-L or a polynucleotide encoding same, compared to a control is indicative of early onset preeclampsia.

31-33. (canceled)

34. A method for diagnosing late onset preeclampsia with or without intrauterine growth restriction (IUGR) comprising comparing levels of a Mtd-L polypeptide of SEQ ID NO. 3 and polynucleotides encoding same in a sample from a subject to the corresponding levels in a control.

35-36. (canceled)

37. A method for monitoring the progression of preeclampsia in an individual, comprising:
(a) contacting an amount of an antibody which binds to a polypeptide according to claim 7, a Mtd-L polypeptide of SEQ ID NO. 3, or a combination thereof, with a sample from the individual so as to form a binary complex comprising the antibody and polypeptide in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the progression of the preeclampsia in the individual.

38-39. (canceled)

40. A method for diagnosing IUGR, comprising comparing levels of PHD1, PHD2, PHD3, or combinations thereof in a sample from a subject to the corresponding levels in a control.

41. (canceled)

42. A method for diagnosing molar pregnancies comprising comparing levels of a polypeptide of claim 7, a Mtd-L polypeptide of SEQ ID NO. 3, or combinations thereof in a sample from a subject to the corresponding levels in a control wherein a significant difference in expression of the polypeptides is indicative of a molar pregnancy.

43. A method according to claim 42 wherein an increase in polypeptide is indicative of a molar pregnancy.

44. (canceled)

45. A method for treating a condition requiring regulation of trophoblast cell death, differentiation, invasion, cell fusion and turnover, or combinations thereof and mediated by a polypeptide encoded by a polynucleotide comprising:
(a) a nucleic acid sequence encoding a polypeptide comprising an amino acid sequence of SEQ ID NO:1,
(b) a nucleic acid sequence of SEQ ID NO: 2;
(c) a nucleic acid sequence complementary to (a) or (b); or
(d) a degenerate form of a nucleic acid sequence of (a) or (b);
said method comprising administering an effective amount of a substance, compound or inhibitor identified in accordance with the method of claim 17.

46. (canceled)

47. A composition comprising one or more polynucleotide of claim 1 and a pharmaceutically acceptable carrier, excipient or diluent.

48-50. (canceled)

51. A test kit for diagnosing a condition requiring regulation of trophoblast cell death, differentiation, invasion, cell fusion and turnover, or a combination thereof comprising a binding agent that interacts with a polypeptide of claim 7.

52. A method of conducting a drug discovery business comprising:
(a) providing one or more assay systems for identifying substances, compounds or inhibitors using the method of to claim 17;
(b) conducting therapeutic profiling of substances, compounds or inhibitors identified in step (a), or further analogs thereof, for efficacy and toxicity in animals; and
(c) formulating a pharmaceutical preparation including one or more substances, compounds or inhibitors identified in step (b) as having an acceptable therapeutic profile.

53. A method of diagnosing or monitoring a condition associated with a polypeptide of claim 7 in a subject by determining the presence of the polypeptide in a sample from the subject.

54. A method according to claim 53, wherein the condition is selected from the group consisting of a condition requiring regulation of trophoblast cell death, differentiation, invasion, and cell fusion and turnover and preeclampsia.

55. A composition comprising one or more polypeptide of claim 7 and a pharmaceutically acceptable carrier, excipient or diluent.


SEQUENCE LISTING (10 sequences)

SEQ ID NO: 1 - protein, 134 aa, Homo sapiens
MIRPSVYRNVARQLHISLQSEPVVTDAFLAVAGHIFSAGITWGKVVSLYAVAAGLAVDCVRQAQPAMVHALVDCLGEFVRKTLATWLRRRGGWTDVLKCVVSTDPGLRSHWLVAALCSFGRFLKAAFFVLLPER

SEQ ID NO: 2 - DNA, 561 bp, Homo sapiens
atgatccggcccagcgtctaccgcaacgtggcgcgtcagctgcacatctccctgcagtctgagcctgtggtgaccgatgcgttcctggccgtggctggccacatcttctctgcaggcatcacgtggggcaaggtggtgtccctgtatgcggtggccgcggggctggccgtggactgtgtgaggcaggcccagcctgccatggtccacgccctcgtggactgcctgggggagttcgtgcgcaagaccctggcaacctggctgcggagacgcggcggatggactgatgtcctcaagtgtgtggtcagcacagaccctggcctccgctcccactggctggtggctgcactctgcagcttcggccgcttcctgaaggctgccttcttcgtgctgctgccagagagatgagctgcccacctggcagtggccgcagcctggccctctgggcccaacgcaggaggccctcagcacccgaacacatcttcctcctccccacccgagcctggagcactctaacctcggagaccccctaagccccgttcctccgcagacccaggccctccg

SEQ ID NO: 3 - protein, 212 aa, Homo sapiens
MEVLRRSSVFAAEIMDAFDRSPTDKELVAQAKALGREYVHARLLRAGLSWSAPERAAPVPGRLAEVCAVLLRLGDELEMIRPSVYRNVARQLHISLQSEPVVTDAFLAVAGHIFSAGITWGKVVSLYAVAAGLAVDCVRQAQPAMVHALVDCLGEFVRKTLATWLRRRGGWTDVLKCVVSTDPGLRSHWLVAALCSFGRFLKAAFFVLLPER

SEQ ID NO: 4 - DNA, 2617 bp, Homo sapiens
ctcgctgcccaggcccccgacgccgcggcaggagccccccaagagcgcgggaagccccgtggacctggcgctcccggctcgggcgtggacggggcgggcgccggggcggggcgcgcgtcctcgcgggtctgaatggaagggtcgaggtcgtcgtcggcggcgagcagatcctgaagccagaactccaccccggcgcccgcgccatgcggcgggagaggtgcggcgccccccacccgcgtcgccgccatggaggtgctgcggcgctcctcggtcttcgccgccgagatcatggacgcctttgaccgctcgcccacagacaaggagctggtggcccaggccaaggcgctgggccgggagtacgtgcacgcgcggctgctgcgcgccggcctctcctggagcgcgcccgagcgtgccgcgccggtcccgggacgcctggctgaggtgtgcgcggtgctgctgcgcctgggcgatgagctggagatgatccggcccagcgtctaccgcaacgtggcgcgtcagctgcacatctccctgcagtctgagcctgtggtgaccgatgcgttcctggccgtggctggccacatcttctctgcaggcatcacgtggggcaaggtggtgtccctgtatgcggtggccgcggggctggccgtggactgtgtgaggcaggcccagcctgccatggtccacgccctcgtggactgcctgggggagttcgtgcgcaagaccctggcaacctggctgcggagacgcggcggatggactgatgtcctcaagtgtgtggtcagcacagaccctggcctccgctcccactggctggtggctgcactctgcagcttcggccgcttcctgaaggctgccttcttcgtgctgctgccagagagatgagctgcccacctggcagtggccgcagcctggccctctgggcccaacgcaggaggccctcagcacccgaacacatcttcctcctccccacccgagcctggagcactctaaccctcggagaccccctaagccccgttcctccgcagacccaggccctccggaaggggtgagtggggaggggctttcctgagcctggagctgggctttggggcagcctgcgaccctccccgcttgtgtcccttctcctgtgatctctgtgttttcccttttctttctggggccaggaagtcagggtcaactcccaggcctcagatgcaggggcccagaacacctgctctcacctgagccccaggtgaaggggcccgggaacacctgctctcacctgagccccaggtgaaggggcccgggaacacctgctctcacctgaaccccaggtgaaggggcccggaacacctgctctcacctgagccccaggtgaaggggcccggaacacctgctctcacctgagccccaggtgaaggggcccgggaacacctgctctcacctgagccccaggtgaaggggcccgggaacacctgctctcacctgaaccccaggtgaaggggcccagaacacctgctctcacctgagccccaggtgaaggggcccggaacacctgctctcacctgagccccaggtgaaggggcccgggaacacctgctctcacctgagcccctggtgaaggggcccggaacacttgctctcacctgagccccaggtgaaggggcccggaacacctgctctcacctgagcccccggtgaaggggcccggaacacttgctctcacctgagccccaggtgaaggggcccggaacacctcctctcacctgagccccaggtgaaggggcccggaacacctcctgtcacctgagccccaggtgaaggggcccgggaacacctctcacctgaacccgggggtcccatcccaggaagaagggccatctcaggacatgagtcctcaggggccctgcacattcaatctgaaggtgaccctggcctggctgaagctggaagagctgtggggactcagcctgtaaacagagcgtaaggttcacatgctggttgcttaatccgtttctggaggaagagtatgacacccacttgtgatggggtccttgtgcggtggggaccggggccggcgggctccaggccagcacacctaacccatggatgtggaacctacggccgagaaggaatgttgcatgagtcggatcccagtccattgtcagtggagggtgagggtgaccccatctgctatttttgtgctcatcctcatacaaccatttggggatgtgcctattagggctccgtaagaactcagatgcctgggaagcccagcccctcaggtgcccccacacacagccttcccttgacgcctacatttctaggcacatgtgaggcatctttcctggagccccgagccagccctgtccctccccagtgcagcatggcactcaggagatacaggctggacatggggcagtcgttctggggaggcctggcctagcagccacccacctgagccctcccggccaggcttcgtgctggggtgggccatgtgccaggacaggagggtcccggcggaaagccagccccggactcatcgtgacattgagatcccactggagggtaggggtggtaataaacttctccaaacgatcgttgtcattttagacagaaaaaaaaaaaaaaaaaa

SEQ ID NO: 5 - protein, 350 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 6 - protein, 271 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGWVCGVLPCRGPRRWHQECAAGFCRCCWSRSWFGISNKIALL

SEQ ID NO: 7 - DNA, 4020 bp, Homo sapiens
caccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaaaaaaaaaaaaaaaaaa

SEQ ID NO: 8 - DNA, 3772 bp, Homo sapiens
caccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaaaaaaaaaaaaaaaaaa

SEQ ID NO: 9 - protein, 271 aa, Homo sapiens; identical to SEQ ID NO: 6

SEQ ID NO: 10 - DNA, 816 bp, Homo sapiens
atgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggatgggtttgtggagttcttccatgtagaggacctagaaggtggcatcaggaatgtgctgctggcttttgcaggtgttgctggagtaggagctggtttggcatatctaataagatagccttactgtaa